(19) United States
(12) Patent Application Publication
(10) Pub. No.: US 2011/0171195 A1
(43) Pub. Date: Jul. 14, 2011

(54) METHODS AND COMPOSITIONS FOR TREATING URINARY TRACT INFECTIONS USING AGENTS THAT MIMIC OR ELEVATE CYCLIC AMP

(75) Inventors: Soman N. Abraham, Chapel Hill, NC (US); Brian L. Bishop, Durham, NC (US); Matthew J. Duncan, Duncan, SC (US); K. Ranga Rama Krishnan, Chapel Hill, NC (US); Jeongmin Song, Chapel Hill, NC (US); Guojie Li, Charlotte, NC (US); David A. Zaas, Chapel Hill, NC (US)

(73) Assignee: Duke University, Durham, NC (US)

(21) Appl. No.: 12/663,959

(22) PCT Filed: Jun. 12, 2008
(86) PCT No.: PCT/US08/66647

§ 371 Date: Sep. 21, 2010

Related U.S. Application Data
(60) Provisional application No. 60/944,182, filed on Jun. 15, 2007.

(51) Int. Cl. A61K 38/45; A61K 31/015; A61K 31/352; A61K 31/496; A61K 31/11; A61K 31/4162; A61K 31/137; A61K 31/7076; A61K 38/17; A61K 31/739; A61K 31/661; A61K 31/7028; A61K 31/4418; A61K 31/341; A61K 31/423; A61K 31/545; A61K 31/43; A61K 31/65; A61P 31/04; A61P 31/00; A61P 3/00

(52) U.S. Cl. 424/94.5; 514/766; 514/455; 514/254.11; 514/699; 514/393; 514/654; 514/653; 514/46; 514/2.4; 514/54; 514/77; 514/25; 514/352; 514/461; 514/375; 514/200; 514/192; 514/154

(57) ABSTRACT

Methods and compositions are provided for treating a urinary tract infection (UTI). The methods involve administering to a subject in need thereof a cAMP elevator or agent that mimics cAMP, particularly a labdane diterpene such as forskolin or a derivative or analog thereof in a therapeutically effective amount to treat a UTI. The methods may further include administration of at least one cAMP elevator in combination with one or more additional active compounds from other classes of therapeutic agents, such as antimicrobial agents or cholesterol lowering drugs. Compositions of the invention include pharmaceutical compositions and kits for treating a UTI in a subject in need thereof that include therapeutically effective amounts of at least two cAMP elevators, particularly where one of the cAMP elevators is a labdane diterpene such as forskolin or a derivative or analog thereof. In particular, the compositions and kits may also include at least one cAMP elevator in combination with one or more additional active compounds from other classes of therapeutic agents, such as antimicrobial agents or cholesterol lowering drugs.

### FEDERALLY SPONSORED RESEARCH OR DEVELOPMENT

This invention was made with government support under grant number R37 DK 50814 awarded by the National Institutes of Health. The United States government has certain rights in the invention.

### FIELD OF THE INVENTION

The presently disclosed subject matter relates to methods and compositions to treat urinary tract infections using agents that mimic or elevate intracellular levels of cAMP.

### BACKGROUND

Urinary tract infections (UTIs) represent one of the most common bacterial infections in humans. Each year at least 4 million patients (mostly women) seek treatment for UTIs, and approximately $1.6 billion will be spent in the diagnosis and treatment of UTIs. Frequent recurrences in healthy adult females after an initial bout of UTI continue to frustrate clinicians. Patients with recurrent UTIs are often subjected to sustained “suppressive” antibiotic therapy, which can be toxic to various organs of the body. Additionally, constant use of antibiotics can result in the development of multiresistant bacteria.

To date, most treatment strategies for UTIs have advocated directly killing bacteria or blocking bacterial adherence to the walls of the bladder. Such treatments typically involve conventional antibiotic therapy, such as through administration of ciprofloxacin, nitrofurantoin, trimethoprim-sulfamethoxamole, levofloxacin, and certain penicillins, such as amoxicillin. Although these approaches have proven somewhat effective, the growing problem of UTI recurrence demonstrates that additional counter measures are required.

There is therefore an urgent need to develop new compounds and methods for the treatment of urinary tract infections.

### SUMMARY OF THE INVENTION

Methods and compositions for treating a urinary tract infection (UTI) are provided. The methods involve administering to a subject in need thereof one or more compounds that increase intracellular levels of cyclic AMP (cAMP elevators) or that mimic the effects of cAMP in a therapeutically effective amount to treat a UTI. Exemplary cAMP elevators for use alone or in combination in these methods include, but are not limited to, adenylate cyclase activators, including, for example, labdane diterpenes (particularly labdane, forskolin, and forskolin derivatives and analogs); agents that inhibit or block the activity of phosphodiesterases (PDE inhibitors); Toll-like receptor ligands; calcium channel activators or calcium activators; protein kinase C (PKC) activators; and adenylate cyclase toxin. Exemplary agents that mimic cAMP include protein kinase A (PKA) activators. The methods may further include administration of at least one cAMP elevator or agent that mimics cAMP in combination with one or more additional active compounds from other classes of therapeutic agents. In one such embodiment, the other class of therapeutic agents includes any agent in use or in development to treat a UTI, including antimicrobial agents such as, for example, antibiotics, and drugs that block bacterial adherence to the bladder wall. In another embodiment, the other class of therapeutic agents includes cholesterol lowering drugs.

Compositions of the invention include pharmaceutical compositions and kits for treating a UTI in a subject in need thereof. In some embodiments, the pharmaceutical compositions and kits include therapeutically effective amounts of at least two cAMP elevators or agents that mimic cAMP, and optionally one or more additional active compounds from other classes of therapeutic agents. In other embodiments, the pharmaceutical compositions and kits include one or more cAMP elevator or agent that mimics cAMP in combination with at least one additional active compound from other classes of therapeutic agents.

### BRIEF DESCRIPTION OF THE DRAWINGS

FIG. 1 shows transmission electron micrographs of uropathogenic E. coli (UPEC) invading superficial bladder epithelial cells (BECs) through fusiform canals. (A) Uninfected bladder epithelium showing scalloped plaques in the lumenal surface as well as in intracellular fusiform vesicles (scale bars=2 μm). (B and C) Attachment of E. coli CI5 to scalloped plaques of the BEC lumenal surface appear to associate with fusion of fusiform vesicles at the attachment site (arrows) (scale bars=4 μm). (D) E. coli CI5 were found in tubular, scallop-shaped canals within superficial BECs (the tubular canal retains a scalloped appearance as sequestered bacteria are no longer in direct contact with the lumenal surface of the bladder; scale bars=4 μm). (E and F) Intracellular E. coli CI5 were also observed in discrete bacterial compartments that did not have a scalloped appearance and that were connected by tethers of collapsed membrane (scale bars=4 μm). (G) The tether appear to consist of a bilayer of membrane, which may be a remnant of the tubular canal (scale bars=4 μm).

FIG. 2 shows fluorescence micrographs of E. coli entry into Rab27b⁺ vesicles within BECs. (A and B) Uninfected mouse bladders were probed for various markers of the bladder epithelium, including uroplakin III (A, green) and Rab27b (B, green), a specific marker for fusiform vesicles. The DNA stain Hoechst-33258 (cyan), distinguished cell layers by nuclear positioning (scale bars=50 μm). (C) Antibodies to E. coli (arrows, left panel) and to Rab27b (arrows, middle panel) on mouse bladders infected with E. coli for 2 hours revealed an association of E. coli CI5 with fusiform vesicles in the superficial epithelium (arrows, right panel) (scale bars=10 μm). (D) 5637 BECs from mouse bladders infected with E. coli for 2 hours and incubated with HcRed-expressing E. coli ORN103(pSH2) showed that intracellular E. coli were encased in membrane enriched with Rab27b-GFP (85% association; right panel) (scale bars=10 μm). (E) Entry of E. coli ORN103(pSH2) into siRNA Rab27b knockdown BECs was significantly reduced after 1 hour of incubation compared to that in siRNA-treated controls. Inset shows RT-PCR of Rab27b levels during invasion: 5637 BECs (1) untreated, (2) treated with control siRNA and (3) treated with Rab27b siRNA. *P<0.05 by unpaired t-test. Error bars represent standard error of the mean.

FIGS. 3A and 3B show that E. coli interact with secretory lysosomes of 5637 BECs in vitro. (A) The presence of secretory lysosomes in 5637 BECs was confirmed through assaying the extracellular media for the activity of f3-hexosaminidase, a constitutive component of secretory lysosomes. Treatment of BECs for 1 hr with forskolin (100 μM), dibut 1-cAMP (1 mM), or calcium ionophore (1 μM) stimulated the BECs to release appreciable levels of β-hexosaminidase. (B) The release of β-hexosaminidase into the extracellular media from E. coli ORN103(pSH2)-infected 5637 BECs was regulatable by either NiCl₂ (2 mM) or H89 (10 μM) in a manner consistent with secretory lysosome exocytosis during E. coli attachment. *P<0.05 by unpaired T-test; Error bars ±1 S.E.M.

FIG. 4 shows that E. coli enters BECs through CD63+ vesicles and bypasses the classical endocytic pathway. (A-F) 5637 BECs were incubated with HcRed-expressing E. coli ORN103(pSH2) or (J-L) HcRed-expressing E. coli ORN103(pSH2) and Alexa Fluor 488-conjugated transferrin. Fluorescent microscopy revealed that intracellular E. coli were encased in membrane enriched with (A-C) CD63 (80% association). (D-F) Even as E. coli endocytosis is in progress, the nascent phagosome acquires CD63 (arrow), presumably through the recruitment and fusion of multiple CD63 positive vesicles (arrowhead). (G-L) Intracellular E. coli exhibited a limited association with the early endosome marker (G-I) EEA1 (8% association) or a marker for early and recycling endosomes, (J-L) AlexaFluor488-transferrin (9% association). Scale bars=10 μm.

FIG. 5 shows E. coli exocytosis from infected BECs. (A) The intracellular population of E. coli ORN103(pSH2) within 5637 BECs declined during the first 24 h and remained stable until 120 h. (B) Following gentamicin treatment, E. coli ORN103(pSH2)-infected 5637 BECs were incubated with antibiotic-free medium. In 3 h, allowing an extracellular population of E. coli (643 CFU) to be cultured; this extracellular population mirrored the decline in intracellular E. coli (576 CFU). (C) One-hour-long exocytosis assays performed on 5637 BECs infected with E. coli ORN103(pSH2), E. coli CI5 or S. enterica SL1344, showing bacterial exocytosis from E. coli-infected 5637 BECs but not from S. enterica-infected 5637 BECs. (D) E. coli ORN103(pSH2) exocytosis into the antibiotic-free medium was reduced by the addition of inhibitors of calcium flux (NiCl₂) and cAMP activity (H89). *P<0.05 by unpaired t-test. Error bars represent S.E.M.

FIG. 6 shows that loss of intracellular E. coli is not due to BEC lysis or bacterial degradation. The possible explanations for the decrease in intracellular E. coli include either (i) E. coli were lysing the host cells and entering the antibiotic media or (ii) the secretory lysosomes contained bactericidal activity. The loss of bacterial viability was not due to a breakdown in 5637 BEC membrane integrity. A trypan blue exclusion assay and lactose dehydrogenase (LDH) release assays demonstrated that 95% of 5637 BECs maintained their membrane integrity and viability after 4 hrs of E. coli infection (data not shown). Additionally, the secretory lysosomes in which the E. coli were harbored did not possess bactericidal activity. Inhibitors of lysosome acidification including NH₄Cl (10 mM, 50 mM) and Bafilomycin (1 μM), which neutralize bactericidal activity within lysosomes, caused no improvement in E. coli ORN103(pSH2) persistence within 5637 BECs.

FIG. 7 shows that forskolin treatment causes exocytosis of fusiform vesicles in BECs and reduces UTI. (A,B) Balb/c mouse bladder sections were examined after intraperitoneal and intravesicular injection of saline (A; 10 mg/kg) or forskolin (B; 100 μM). Each section was probed with antibodies to Rab27b and examined using bright-field (A, B, left panels) and immunofluorescence (A, B, middle panels) microscopy. The results were also merged (A, B, right panels). Arrowheads delineate the superficial epithelium. Scale bar, 50 μm. (C) Mice were infected intravesicularly with either E. coli CI5 or S. enterica SL1344 and then treated with forskolin 2 h after infection (intraperitoneally, 10 mg/kg; by catheter, 100 μM). Excised bladders from forskolin- or saline-treated mice were homogenized and plated for intracellular bacterial CFU. (D) Intravesicular forskolin treatment (100 μM) reduced E. coli CI5 colonization of Balb/c mouse bladders when compared with that of saline-treated controls. (E) Multiple intraperitoneal injections of forskolin (10 mg/kg; 6, 24 and 48 h after infection) reduced E. coli CI5 colonization of C3H/HeJ mouse bladders when compared to that in saline controls. (F) IL-6 levels were examined in urine collected from C3H/HeJ mice before forskolin treatment (6 h after infection) and 18 h after forskolin treatment (24 h after infection). IL-6 levels were significantly different after forskolin treatment, compared to levels in infected but untreated control bladders. *P<0.05 by unpaired t-test. Error bars represent S.E.M.

FIG. 8 is a graphical representation of results from a gentamicin protection assay demonstrating that forskolin treatment negatively affected UPEC invasion into BECs.

FIG. 9 is a graphical representation showing that forskolin reduced the CI5 E. coli load within the bladder after 1 hr of treatment.

FIG. 10 is a graphical representation showing that bacterial lipopolysccharide (LPS), a Toll-like Receptor 4 (TLR4) ligand, elicted a clear and measurable increase in intracellular cAMP in human bladder epithelial cells.

FIG. 11 shows the results of caffeine, a nonspecific PDE inhibitor, on intracellular cAMP in human bladder epithelial cells.

FIG. 12 shows the results of papaverine, a PDE inhibitor, on intracellular cAMP in human bladder epithelial cells.

FIG. 13 shows the results of isobutylmethylxanthine (IBMX), a nonspecific PDE inhibitor, on intracellular cAMP in human bladder epithelial cells.

FIG. 14 shows the results of erythro-9-(2-hydroxy-3-nonyl)adenine (EHNA), a PDE2 inhibitor, on intracellular cAMP in human bladder epithelial cells.

FIG. 15 shows the results of rolipram, a PDE4 inhibitor, on intracellular cAMP in human bladder epithelial cells.

FIG. 16 shows the results of zaprinast, a PDE5/6 inhibitor, on intracellular cAMP in human bladder epithelial cells.

FIG. 17 shows the results of cilostamide (N-Cyclohexyl-N-methyl-4-(1,2-dihydro-2-oxo-6-quinolyloxy)butyramide), a PDE3 inhibitor, on intracellular cAMP in human bladder epithelial cells.

FIG. 18 shows the results of phorbol ester (PMA), a PKC inducer, on intracellular cAMP in human bladder epithelial cells.

FIG. 19 shows the results of lipopolysccharide (LPS), a Toll-like receptor 4 (TLR4) ligand, on bacterial exoyctosis from E. coli (CI5) infected BECs as compared to control infected BECs.

### DETAILED DESCRIPTION

The present invention provides methods and compositions for treating a urinary tract infection (UTI) in a subject in need thereof. The methods comprise administering to a subject in need thereof at least one compound that increases intracellular levels of cyclic AMP (herein after referred to as a “cAMP elevator”) or that mimics the effects of cAMP in a therapeutically effective amount to treat a UTI. In some embodiments, the methods comprise administration of one or more cAMP elevator or agent that mimics cAMP in combination with at least one other active compound from other classes of therapeutic agents. Pharmaceutical compositions and kits for treating a UTI in a subject in need thereof are also provided. These pharmaceutical compositions and kits comprise therapeutically effective amounts of at least one cAMP elevator or agent that mimics cAMP and one or more additional active compounds from other classes of therapeutic agents. In one such embodiment, the pharmaceutical compositions and kits include at least one cAMP elevator or agent that mimics cAMP and an antimicrobial agent or a cholesterol lowering drug. In other embodiments, the pharmaceutical compositions and kits comprise at least two cAMP elevators or agents that mimic cAMP, and optionally comprise one or more additional active compounds from other classes of therapeutic agents. In particular embodiments of the methods and compositions of the invention, cAMP elevators include, but are not limited to, adenylate cyclase activators such as labdane diterpenes (particularly labdane, forskolin, and forskolin derivatives and analogs) and other adenylate cyclase activators described below, agents that inhibit or block the activity of cAMP and/or cGMP phosphodiesterases (PDE inhibitors), Toll-like receptor ligands, calcium channel activators or calcium activators, protein kinase C (PKC) activators, and adenylate cyclase toxin. Agents that mimic cAMP include, but are not limited to, protein kinase A (PKA) activators.

A UTI is an inflammatory process occurring in the urinary tract that occurs when microorganisms (usually Escherichia coli) enter through the urethra. These infections can happen anywhere along the urinary tract, i.e., the kidneys, the ureters (the tubes that take urine from each kidney to the bladder), the bladder, or the urethra (the tube that empties urine from the bladder to the outside). However, most urinary tract infections occur in the lower urinary tract, which includes the bladder and urethra. Cystitis is caused when the normally sterile lower urinary tract is infected by bacteria and becomes inflamed. Urinary tract infections can be acute (i.e., a single occurrence), or chronic. Chronic UTIs include repeated episodes of cystitis (more than 2 in 6 months), or urinary tract infection that does not respond to the usual treatment or that lasts longer than 2 weeks.

The methods and compositions of the present invention are based upon the discovery that cAMP elevators or agents that mimic cAMP, particularly the adenylate cyclase activators classified as labdane diterpenes such as forskolin and derivatives and analogs thereof, alone or in combination with additional therapeutic agents, are useful to treat UTIs, including acute or chronic (recurrent) UTIs. To date, most treatment strategies for UTIs have advocated directly killing bacteria or blocking bacterial adherence to the walls of the bladder. These approaches have had limited efficacy, and the recurrence of UTIs is a growing problem that indicates the need for additional counter measures. As described more fully in the Experimental section below, cAMP elevators or agents that mimic cAMP cause the collapse of intracellular compartments that harbor bacteria within the cells of the bladder walls. Once expelled from their intracellular niches, these bacteria are susceptible to clearance by either the flushing actions of urine or treatment with an antimicrobial agent such as an antibiotic.

Accordingly, in one embodiment the present invention relates to a method for treating a UTI comprising administering to a subject in need thereof a therapeutically effective amount of at least one cAMP elevator or agent that mimics cAMP.

The term “cAMP elevator” as used herein refers to an agent that increases intracellular levels of cAMP beyond the background physiological intracellular level. cAMP is synthesized from ATP by the enzyme adenylate cyclase and is degraded into AMP by cAMP phosphodiesterases. cAMP elevators therefore include agents that activate or enhance the activity of adenylate cyclase (hereinafter referred to as “adenylate cyclase activators”), agents that increase the availability of adenylate cyclase, and agents that inhibit or block the activity of cAMP and/or cGMP phosphodiesterases (hereinafter referred to as “PDE inhibitors”). Other representative cAMP elevators include, but are not limited to, the Toll-like receptor ligands, calcium channel activators or calcium activators, protein kinase C (PKC) activators, and adenylate cyclase toxin. These classes of cAMP elevators are described in more detail herein below. In a particular embodiment, the cAMP elevator is an adenlyate cyclase activator, more particularly, a labdane diterpene such as forskolin or a derivative or analog thereof. In another particular embodiment, the cAMP elevator is a Toll-like receptor ligand.

The term “agent that mimics cAMP” as used herein refers to an agent that produces physiological effects similar to endogenous cAMP such as, for example, activating protein kinase A (PKA). Accordingly, agents that mimic cAMP include, for example, PKA activators as described in more detail herein below.

In accordance with the methods of the present invention, a subject in need of treatment for a UTI may be administered a therapeutically effective amount of a single cAMP elevator or agent that mimics cAMP. Alternatively, the subject may be administered therapeutically effective amounts of two or more cAMP elevators or agents that mimic cAMP. When multiple cAMP elevators or agents that mimic cAMP are to be administered to a subject to treat a UTI, the cAMP elevators or agents that mimic cAMP can be chosen from the same class (or type) of cAMP elevators or agents that mimic cAMP, or can be chosen from two or more classes of cAMP elevators or agents that mimic cAMP. Thus, for example, where at least two cAMP elevators or agents that mimic cAMP are to be administered, they can be selected from one or more of the following non-limiting examples of classes of cAMP elevators or agents that mimic cAMP: adenylate cyclase activators, PDE inhibitors, Toll-like receptor ligands, calcium channel activators or calcium activators, protein kinase A activators, protein kinase C activators, and adenylate cyclase toxin, as described herein below.

Thus, the present invention also relates to a combination therapy for the treatment of a UTI in which a therapeutically effective amount of each of two or more cAMP elevators or agents that mimic cAMP is administered to a subject in need thereof. In some embodiments, the combination therapy comprises administering a therapeutically effective amount of each of two or more adenylate cyclase activators. In other embodiments, the combination therapy comprises administering a therapeutically effective amount of an adenylate cyclase activator in combination with at least one additional cAMP elevator or agent that mimics cAMP selected from the group consisting of a PDE inhibitor, a Toll-like receptor ligand, a calcium channel activator or calcium activator, a protein kinase A activator, a protein kinase C activator, and adenylate cyclase toxin, as described more fully herein below.

Where the combination therapy comprises the administration of a combination of two or more adenylate cyclase activators, in one embodiment the adenylate cyclase activators are selected from the group consisting of the labdane diterpenes, which are described in more detail below. In some of these embodiments, the first adenylate cyclase activator is forskolin or a derivative or analog thereof. Exemplary forskolin derivatives and analogs are disclosed herein below, and include, but are not limited to, the water soluble forskolin derivative known as NKH477 (colforsin daropate hydrochloride). In one embodiment the first adenylate cyclase activator is forskolin, and the second adenylate cyclase activator is NKH477.

In other embodiments, at least one of the two or more adenylate cyclase activators is a labdane diterpene, and the remaining adenylate cyclase activator(s) is (are) selected from the group consisting of a G-protein coupled receptor agonist, a G-protein activator, the pyrazole derivative A02011-1 (see Yu et al. (1995) Br. J. Pharmacol. 114:1227-1235), and benzyloxybenzaldehyde and analogs thereof such as those disclosed in Chang et al. (2001) Bioorg. Med. Chem. Lett. 11:1971-1974. Exemplary G-protein coupled receptor agonists and G-protein activators are described more fully herein below.

Where the combination therapy for the treatment of a UTI comprises administering a therapeutically effective amount of each of two or more cAMP elevators or agents that mimic cAMP to a subject in need thereof, in a particular embodiment the method comprises administering a therapeutically effective amount of an adenylate cyclase activator in combination with a therapeutically effective amount of a PDE inhibitor. Without being bound by any theory or mechanism of action, this type of combination therapy advantageously administers a class of molecules that inhibit the otherwise rapid in vivo degradation of cAMP by PDE while also administering a class of molecules that accelerate intracellular cAMP production (i.e., adenylate cyclase activator). Such combination therapy could therefore be of benefit in sustaining elevated levels of cAMP, once generated. Again, without being bound by theory or mechanism of action, PDE inhibitors when combined with activators of adenylate cyclase can result in accumulation of much higher levels of cAMP within cells than can be obtained with administration of either of these classes of cAMP elevators alone. Exemplary PDE inhibitors are described herein below. In some embodiments, the adenylate cyclase activator that is to be administered in combination with a PDE inhibitor is a labdane diterpene, such as those described herein below. In one such embodiment, the labdane diterpene to be used in this type of combination therapy for a UTI is forskolin or a derivative or analog thereof.

Where the combination therapy for the treatment of a UTI comprises administering a therapeutically effective amount of each of two or more cAMP elevators or agents that mimic cAMP to a subject in need thereof, in a particular embodiment the method comprises administering a therapeutically effective amount of an adenylate cyclase activator in combination with a therapeutically effective amount of another type of cAMP elevator or agent that mimics cAMP selected from the group consisting of a Toll-like receptor ligand, a calcium channel activator or calcium activator, a protein kinase A activator, a protein kinase C activator, and adenylate cyclase toxin. Again, without being bound by theory or mechanism of action, this type of combination therapy advantageously provides for multiple modes of accelerating intracellular production of cAMP to facilitate accumulation of elevated levels of intracellular cAMP and/or to provide agents that mimic cAMP, thereby enhancing the collapse of intracellular compartments that harbor bacteria within the cells of the bladder walls. Suitable Toll-like receptor ligands, calcium channel activators or calcium activators, protein kinase A and C activators, and the adenylate cyclase toxin are described in more detail herein below. In some embodiments, the adenylate cyclase activator is a labdane diterpene, such as those described herein below. In one such embodiment, the labdane diterpene to be used in this type of combination therapy for a UTI is forskolin or a derivative or analog thereof.

The present invention also provides a combination therapy for the treatment of a UTI in which a therapeutically effective amount of at least one cAMP elevator or agent that mimics cAMP is administered in combination with a therapeutically effective amount of one or more active compounds from other classes of therapeutic agents. In this manner, combination therapy for a UTI in accordance with the methods of the present invention contemplates the administration of one or more cAMP elevators or agent that mimics cAMP in combination with any agent in use or in development to treat a UTI, including other types of therapeutic agents that are not involved in regulation of cAMP intracellular levels. One exemplary class of therapeutic agents to be used in this type of combination therapy is antimicrobial agents, including but not limited to antibiotics and drugs that block bacterial adherence to the bladder wall, as described in more detail herein below. Other exemplary classes of therapeutic agents to be used in this type of combination therapy include drugs that disrupt or chelate cholesterol (“cholesterol lowering drugs”), as described in more detail herein below.

Thus, in one embodiment, the invention relates to a method for treating a UTI comprising administering to a subject in need thereof a therapeutically effective amount of a cAMP elevator or agent that mimics cAMP, particularly labdane diterpenes such as forskolin or a derivative or analog thereof, in combination with a therapeutically effective amount of an antimicrobial agent that is suitable for treating a UTI. Without being bound by any theory or mechanism of action, it is believed that combination therapy with one or more cAMP elevators or agent that mimics cAMP and at least one antimicrobial agent advantageously provides for elevated levels of intracellular cAMP to facilitate the expulsion of bacteria from their intracellular compartments within the cells of the bladder walls, and a source of antimicrobial agent to clear (kill) the bacteria released from these compartments. Exemplary antimicrobial agents include, but are not limited to, antibiotics, drugs that block bacterial adherence to the bladder wall, and/or any other antimicrobials commonly used to treat UTIs as described more fully below.

In another embodiment, the invention relates to a method for treating a UTI comprising administering to a subject in need thereof a therapeutically effective amount of a cAMP elevator or agent that mimics cAMP, particularly labdane diterpenes such as forskolin or a derivative or analog thereof, in combination with a therapeutically effective amount of a cholesterol lowering drug. Without being bound by any theory or mechanism of action, it is believed that combination therapy with one or more cAMP elevators or agents that mimic cAMP and at least one cholesterol lowering drug advantageously provides for elevated levels of intracellular cAMP or agent that mimics the effects of cAMP to facilitate the expulsion of bacteria from their intracellular compartments within the cells of the bladder walls, and a source of agent to reduce intracellular carriage of bacteria. Exemplary cholesterol lowering drugs for use in the methods of the invention are described more fully below.

In yet other embodiments, the invention relates to a method for treating a UTI comprising administering to a subject in need thereof a therapeutically effective amount of each of two or more cAMP elevators or agents that mimic cAMP, particularly an adenylate cyclase activator in combination with an additional type of cAMP elevator or agent that mimics cAMP, further in combination with a therapeutically effective amount of an antimicrobial agent and/or a cholesterol lowering drug. In some of these embodiments, the adenylate cyclase activator is a labdane diterpene such as forskolin or a derivative or analog thereof and the additional type of cAMP elevator or agent that mimics cAMP is selected from the group consisting of a PDE inhibitor, a Toll-like receptor ligand, a calcium channel activator or calcium activator, a protein kinase A activator, a protein kinase C activator, and adenylate cyclase toxin. In still other embodiments, combination therapy for a UTI using two or more adenylate cyclase activators as described herein above further comprises administration of a therapeutically effective amount of an antimicrobial agent and/or a cholesterol lowering drug.

Where multiple therapeutic agents are to be administered in combination with each other to treat a UTI, the administration of these agents can occur concurrently (simultaneously) or sequentially (consecutively) in any order. For concurrent administration of multiple therapeutic agents (for example, two or more cAMP elevators or agents that mimic cAMP, and optionally another active compound such as an antimicrobial agent and/or a cholesterol lowering drug, or a single cAMP elevator or agent that mimics cAMP and another active compound such as an antimicrobial agent and/or a cholesterol lowering drug), the multiple agents may be formulated either within a single pharmaceutical composition or within separate pharmaceutical compositions (for example, one formulation comprising the cAMP elevator(s) or agent that mimics cAMP and one formulation comprising the other active compound. For sequential administration, each therapeutic agent can be formulated in its own pharmaceutical composition, each of which is to be administered sequentially, in any order; alternatively, two or more of the therapeutic agents can be formulated together (for example, two cAMP elevators or agents that mimic cAMP) as a first pharmaceutical composition, with a third therapeutic agent (for example, an antimicrobial agent and/or a cholesterol lowering drug) being formulated as a second pharmaceutical composition, so that the first and second pharmaceutical compositions are administered sequentially, in either order.

The terms “treat” or “treatment” as used herein refer to the application or administration of one or more cAMP elevators or agents that mimic cAMP and/or one or more additional active compounds to a subject having a UTI or symptom of a UTI, and where the purpose is to cure, heal, alleviate, relieve, alter, remedy, ameliorate, improve, or affect the UTI or any associated symptoms of the UTI.

The term “subject” refers to any organism to which the presently disclosed treatment methods and pharmaceutical compositions can be administered. In specific embodiments, a subject is a mammal. In other embodiments, a subject is a primate, a human, a domestic animal, or an agricultural animal. A subject can include a human subject for medical purposes, such as treatment of a condition or disease, or an animal subject for medical, veterinary purposes, or developmental purposes. Suitable animal subjects include mammals and avians. The term “avian” as used herein includes, but is not limited to, chickens, ducks, geese, quail, turkeys, and pheasants. The term “mammal” as used herein includes, but is not limited to, primates, e.g, humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. It has been reported that UTI's account for between 5-10% of canine and 0.1-1% of feline veterinary visits (see, e.g., Dunning and Stonehewer (2002) In Practice 24:418-432). Thus, in one embodiment of the present invention the subject is a mammal such as a domestic cat or dog. In another embodiment the subject is a human. Further, a “subject” can include a patient afflicted with or suspected of being afflicted with a condition or disease. Thus, the terms “subject” and “patient” are used interchangeably herein.

A therapeutically effective amount of a cAMP elevator or agent that mimics cAMP or additional active compound within the methods and compositions of the present invention typically ranges from about 1 μg/kg to about 500 mg/kg, about 10 μg/kg to about 500 mg/kg, about 100 μg/kg to about 500 mg/kg, about 1 mg/kg to about 500 mg/kg, about 1 mg/kg to about 400 mg/kg, about 1 mg/kg to about 300 mg/kg, about 1 mg/kg to about 200 mg/kg, about 1 mg/kg to about 100 mg/kg, about 1 mg/kg to about 75 mg/kg, about 1 mg/kg to about 50 mg/kg, or about 1 mg/kg to about 25 mg/kg. In another embodiment, the therapeutically effective dose of a cAMP elevator or additional active compound is an amount of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 110 mg/kg, about 120 mg/kg, about 125 mg/kg, about 130 mg/kg, about 140 mg/kg, about 150 mg/kg, about 160 mg/kg, about 170 mg/kg, about 175 mg/kg, about 180 mg/kg, about 190 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 425 mg/kg, about 450 mg/kg, about 475 mg/kg, to about 500 mg/kg.

For particular agents with greater toxicity profiles, one of skill in the art will appreciate that the therapeutically effective amount may be even lower to achieve a nontoxic but therapeutically effective dose, for example from about 1 pg/kg to about 1 μg/kg, about 50 pg/kg to about 1 μg/kg, about 100 pg/kg to about 1 μg/kg, about 500 pg/kg to about 1 μg/kg, about 1 ng/kg to about 1 mg/kg, about 50 ng/kg to about 1 mg/kg, about 100 ng/kg to about 1 mg/kg, about 500 ng/kg to about 1 mg/kg, about 1 μg/kg to about 1 mg/kg, about 50 μg/kg to about 1 mg/kg, about 100 μg/kg to about 1 mg/kg, or about 500 μg/kg to about 1 mg/kg. In such embodiments, the therapeutically effective dose of a cAMP elevator or additional active compound is an amount of about 1 pg/kg, about 5 pg/kg, about 10 pg/kg, about 20 pg/kg, about 30 pg/kg, about 40 pg/kg, about 50 pg/kg, about 100 pg/kg, about 200 pg/kg, about 300 pg/kg, about 400 pg/kg, about 500 pg/kg, about 600 pg/kg, about 700 pg/kg, about 800 pg/kg, about 900 pg/kg, about 1 ng/kg, about 5 ng/kg, about 10 ng/kg, about 20 ng/kg, about 30 ng/kg, about 40 ng/kg, about 50 ng/kg, about 100 ng/kg, about 200 ng/kg, about 300 ng/kg, about 400 ng/kg, about 500 ng/kg, about 600 ng/kg, about 700 ng/kg, about 800 ng/kg, about 900 ng/kg, about 1 μg/kg, about 5 μg/kg, about 10 μg/kg, about 20 μg/kg, about 30 μg/kg, about 40 μg/kg, about 50 μg/kg, about 100 μg/kg, about 200 μg/kg, about 300 μg/kg, about 400 μg/kg, about 500 μg/kg, about 600 μg/kg, about 700 μg/kg, about 800 μg/kg, about 900 μg/kg, about 1 mg/kg, and other such values between about 1 pg/kg and about 1 mg/kg.

As used herein, the term “about,” when referring to a value is meant to encompass variations of, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Where the subject is a human subject, the dosage levels are based upon a body weight of approximately 70 kg. It will be understood, however, that the specific dose level and frequency of dosage for any particular subject may be varied and will depend upon a variety of factors including body weight, age, general health, sex, and diet of the subject, the metabolic stability and length of action of the administered compound, mode and time of administration, rate of excretion, drug combination, and severity of the particular UTI.

The toxicity and therapeutic efficacy of a cAMP elevator or agent that mimics cAMP or additional active compound within the methods and compositions of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected urinary tract tissue to minimize potential damage to uninfected cells and, thereby, reduce side effects. The dosage lies preferably within a range of circulating concentrations that include the ED₅₀ with little or no toxicity, and can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

The cAMP elevator(s), agent(s) that mimic cAMP, or additional active compound(s) can be formulated according to known methods to prepare pharmaceutically useful compositions, and may be administered to a subject by any mode of administration, including oral, intravesicular (into the bladder), intraurethral (e.g., through a catheter), transurethral, vaginal, rectal, topical, nasal, ophthalmic, or parenteral (including intraperitoneal, intravenous, subcutaneous, or intramuscular injection) administration. Suitable formulations and their appropriate carrier vehicles are described, for example, in Remington's Pharmaceutical Sciences (18^{th} ed.; Mack Publishing Company, Eaton, Pa., 1990), herein incorporated by reference.

Accordingly, in one embodiment, the present invention relates to a pharmaceutical composition for treating a UTI in a subject in need thereof that includes two or more cAMP elevators or agents that mimic cAMP in therapeutically effective amounts for treating a UTI, and a pharmaceutically acceptable carrier. In some embodiments, this pharmaceutical composition further comprises one or more additional active compounds from other classes of therapeutic agents. In another embodiment, the present invention relates to a pharmaceutical composition for treating a UTI in a subject in need thereof that includes at least one cAMP elevator or agent that mimics cAMP and one or more additional active compounds from other classes of therapeutic agents, each of which is present in a therapeutically effective amount for treating a UTI in a subject in need thereof, and a pharmaceutically acceptable carrier. As used herein the term “pharmaceutically acceptable carrier” includes solvents, dispersion media, coatings, antibacterial and antifungal agents, isotonic and absorption delaying agents, and the like, compatible with pharmaceutical administration. In some embodiments, the additional active compound is any agent in use or in development to treat a UTI, including antimicrobial agents such as antibiotics, and drugs that block bacterial adherence to the bladder wall, as described elsewhere herein. In other embodiments, the additional active compound is a cholesterol lowering drug as described elsewhere herein.

Thus, in one embodiment, the invention provides a pharmaceutical composition for treating a UTI comprising therapeutically effective amounts of two or more cAMP elevators or agents that mimic cAMP and a pharmaceutically acceptable carrier. In some of these embodiments, the two or more cAMP elevators or agents that mimic cAMP within the pharmaceutical composition are from the same class (or type) of cAMP elevators or agents that mimic cAMP; in other embodiments, the two or more cAMP elevators or agents that mimic cAMP are from two or more classes of cAMP elevators or agents that mimic cAMP. The two or more cAMP elevators or agents that mimic cAMP within the pharmaceutical composition are thus selected from one or more of the following non-limiting examples of classes of cAMP elevators or agents that mimic cAMP: adenylate cyclase activators, PDE inhibitors, Toll-like receptor ligands, calcium channel activators or calcium activators, protein kinase A activators, protein kinase C activators, and adenylate cyclase toxin.

Where the composition comprising two or more cAMP elevators or agents that mimic cAMP comprises a combination of two or more adenylate cyclase activators, in one embodiment the adenylate cyclase activators are selected from the group consisting of the labdane diterpenes, which are described in more detail below. In some of these embodiments, the first adenylate cyclase activator is forskolin or a derivative or analog thereof. Exemplary forskolin derivatives and analogs are disclosed herein below, and include, but are not limited to, the water soluble forskolin derivative known as NKH477 (colforsin daropate hydrochloride). In one embodiment the first adenylate cyclase activator is forskolin, and the second adenylate cyclase activator is NKH477. In another embodiment, at least one of the adenylate cyclase activators is a labdane diterpene, and the remaining adenylate cyclase activator(s) is (are) selected from the group consisting of a G-protein coupled receptor agonist, a G-protein activator, the pyrazole derivative A02011-1 (see Yu et al. (1995) Br. J. Pharmacol. 114:1227-1235), and benzyloxybenzaldehyde and analogs thereof such as those disclosed in Chang et al. (2001) Bioorg. Med. Chem. Lett. 11:1971-1974. Exemplary G-protein coupled receptor agonists and G-protein activators are described more fully herein below.

In other embodiments, where the composition comprises two or more cAMP elevators or agents that mimic cAMP in therapeutically effective amounts for treating a UTI, at least one of the cAMP elevators is an adenylate cyclase activator, and at least one of the remaining cAMP elevator(s) is a PDE inhibitor. In further embodiments, where the composition comprises two or more cAMP elevators or agents that mimic cAMP, at least one of the cAMP elevators is an adenylate cyclase activator, and at least one of the remaining cAMP elevator(s) is a Toll-like receptor ligand, a calcium channel activator or calcium activator, a protein kinase A activator, a protein kinase C activator, or adenylate cyclase toxin. In particular embodiments, the adenylate cyclase activator within such compositions is a labdane diterpene, particularly forskolin or a derivative or analog thereof.

Each of these pharmaceutical compositions comprising two or more cAMP elevators or agents that mimic cAMP in therapeutically effective amounts for treating a UTI can optionally comprise an additional active compound from another class of therapeutic agents. In some embodiments, the additional active compound is from the class of agents that are suitable for treating a UTI in a subject in need thereof, including antimicrobial agents and/or cholesterol lowering drugs as described elsewhere herein.

In another embodiment, the pharmaceutical compositions of the present invention for treating a UTI comprise therapeutically effective amounts of a cAMP elevator or agent that mimics cAMP, an additional active compound from another class of therapeutic agents, and a pharmaceutically acceptable carrier. The cAMP elevator or agent that mimics cAMP can be any of the cAMP elevators or agents that mimic cAMP described elsewhere herein, including, for example, an adenylate cyclase activator, a PDE inhibitor, a Toll-like receptor ligand, a calcium channel activator or calcium activator, protein kinase A activators, protein kinase C activators, and adenylate cyclase toxin. In some embodiments, the additional active compound is from the class of agents that are suitable for treating a UTI in a subject in need thereof, including antimicrobial agents and/or cholesterol lowering drugs as described elsewhere herein. In some of these embodiments, the labdane diterpene within these pharmaceutical compositions is forskolin or a derivative or analog thereof.

As one of ordinary skill in the art would appreciate, the presently disclosed pharmaceutical compositions are formulated to be compatible with their intended route of administration. Solutions or suspensions used for parenteral (e.g., intravenous), intramuscular, intradermal, or subcutaneous application can include the following components: a sterile diluent such as water for injection, saline solution, fixed oils, polyethylene glycols, glycerine, propylene glycol or other synthetic solvents; antibacterial agents, such as benzyl alcohol or methyl parabens; antioxidants, such as ascorbic acid or sodium bisulfite; chelating agents, such as ethylenediaminetetraacetic acid; buffers, such as acetates, citrates or phosphates; and agents for the adjustment of tonicity, such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The composition should be sterile and should be fluid to the extent that easy syringability exists. Preferred pharmaceutical compositions are stable under the conditions of manufacture and storage and should be preserved against the contaminating action of microorganisms, such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants.

Oral formulations generally include an inert diluent or an edible carrier. For the purpose of oral therapeutic administration, the cAMP elevator or agent that mimics cAMP and one or more additional active compounds can be incorporated with excipients and used in the form of tablets, troches, or capsules, e.g., gelatin capsules. Pharmaceutically compatible binding agents, and/or adjuvant materials can be included as part of the composition. The tablets, pills, capsules, troches, and the like can contain any of the following ingredients, or compounds of a similar nature: a binder, such as microcrystalline cellulose, gum tragacanth or gelatin; an excipient, such as starch or lactose, a disintegrating agent, such as alginic acid, Primogel, or corn starch; a lubricant, such as magnesium stearate or Sterotes; a glidant, such as colloidal silicon dioxide; a sweetening agent, such as sucrose or saccharin; or a flavoring agent, such as peppermint, methyl salicylate, or orange flavoring. Formulations for oral delivery can advantageously incorporate agents to improve stability within the gastrointestinal tract and/or to enhance absorption.

For administration by inhalation, the presently disclosed formulations and compositions are preferably delivered in the form of an aerosol spray from a pressured container or dispenser which contains a suitable propellant, e.g., a gas such as carbon dioxide, or a nebulizer. Liquid aerosols, dry powders, and the like, also can be used.

Systemic administration of the presently disclosed compositions also can be by transmucosal or transdermal means. For transmucosal or transdermal administration, penetrants appropriate to the barrier to be permeated are used in the composition. Such penetrants are generally known in the art, and include, for example, for transmucosal administration, detergents, bile salts, and fusidic acid derivatives. Transmucosal administration can be accomplished through the use of nasal sprays or suppositories (urethral or rectal). For transdermal administration, the active compounds are formulated into ointments, salves, gels, or creams as generally known in the art.

The presently disclosed compositions also can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials also can be obtained commercially from Alza Corporation and Nova Pharmaceuticals, Inc. Liposomal suspensions (including liposomes targeted to infected cells with monoclonal antibodies to viral antigens) also can be used as pharmaceutically or cosmetically acceptable carriers. Such suspensions can be prepared according to methods known to those skilled in the art, for example, as described in U.S. Pat. No. 4,522,811, which is incorporated herein by reference in its entirety.

It is advantageous to formulate oral or parenteral formulations in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

The present invention also relates to a packaged kit that contains: 1) at least two cAMP elevators or agents that mimic cAMP, and optionally one or more additional active compounds from other classes of therapeutic agents, each in therapeutically effective amounts to treat a UTI; or 2) at least one cAMP elevator or agent that mimics cAMP and one or more additional active compounds from other classes of therapeutic agents, each in therapeutically effective amounts to treat a UTI. The packaged kit will also include a container for housing the active agents during storage and prior to use, and instructions for carrying out drug administration in a manner effective to treat a UTI. The instructions will typically be written instructions on a package insert and/or on a label. The cAMP elevator(s) or agent(s) that mimic cAMP and one or more additional active compounds may be formulated in any suitable pharmaceutical composition as described elsewhere herein.

Thus, in one embodiment, the invention relates to packaged kits for a subject to use in the treatment of a UTI comprising: a) a first component comprising a labdane diterpene; b) a second component comprising an adenylate cyclase activator selected from the group consisting of a G-protein coupled receptor agonist, a G-protein activator, the pyrazole derivative A02011-1 (see Yu et al. (1995) Br. J. Pharmacol. 114:1227-1235), or benzyloxybenzaldehyde and analogs thereof such as those disclosed in Chang et al. (2001) Bioorg. Med. Chem. Lett. 11:1971-1974. Exemplary G-protein coupled receptor agonists and G-protein activators are described more fully herein below. In some of these kits, the labdane diterpene is forskolin or a derivative or analog thereof.

In other embodiments, the invention relates to packaged kits for a subject to use in the treatment of a UTI comprising: a) a first component comprising an adenylate cyclase activator; b) a second component comprising a PDE inhibitor; and c) instructions for carrying out drug administration of said first and second components in a manner effective to treat said UTI. Exemplary adenylate cyclase activators and PDE inhibitors are described elsewhere herein. In some of these kits, the adenylate cyclase activator is a labdane diterpene such as forskolin or a derivative or analog thereof.

In yet other embodiments, the invention relates to packaged kits for a subject to use in the treatment of a UTI comprising: a) a first component comprising an adenylate cyclase activator; b) a second component comprising a cAMP elevator or agent that mimics cAMP selected from the group consisting of a Toll-like receptor ligands, a calcium channel activator or calcium activator, a protein kinase A activator, a protein kinase C activator, and adenylate cyclase toxin; and c) instructions for carrying out drug administration of said first and second components in a manner effective to treat said UTI. Suitable adenylate cyclase activators, Toll-like receptor ligands, calcium channel activators or calcium activators, protein kinase A activators, protein kinase C activators, and the adenylate cyclase toxin for use in these kits are described in more detail elsewhere herein. In some of these embodiments, the labdane diterpene within these pharmaceutical compositions is forskolin or a derivative or analog thereof. In some of these kits, the adenylate cyclase activator is a labdane diterpene such as forskolin or a derivative or analog thereof.

Each of these particular kits of the present invention comprising at least two cAMP elevators or agents that mimic cAMP can optionally comprise a third component that is an additional active compound from another class of therapeutic agents, in which case, the kit includes instructions for carrying out drug administration of the first, second, and third components in a manner effective to treat the UTI. In some embodiments, the additional active compound is an agent suitable for treating a UTI in a subject in need thereof, including the antimicrobial agents described elsewhere herein. In other embodiments, the additional active compound is a cholesterol lowering drug.

In still other embodiments, the invention relates to packaged kits for a subject to use in the treatment of a UTI comprising: a) a first component comprising a cAMP elevator or agents that mimic cAMP; b) a second component comprising an additional active compound from another class of therapeutic agents; and c) instructions for carrying out drug administration of said first and second components in a manner effective to treat said UTI. These kits can comprise any of the cAMP elevators or agents that mimic cAMP described elsewhere herein, including, for example, adenylate cyclase activators, PDE inhibitors, Toll-like receptor ligands, calcium channel activators or calcium activators, protein kinase A activators, protein kinase C activators, and adenylate cyclase toxin. In some embodiments, the cAMP elevator within these kits of the invention is an adenylate cyclase activator and the additional active compound is an antimicrobial agent and/or a cholesterol lowering drug. In one such embodiment, the kit comprises as a first component a labdane diterpene, such as forskolin or derivative or analog thereof, and as a second component an antimicrobial agent and/or a cholesterol lowering drug.

In one embodiment, the first and second (and/or third) components of these kits are contained in the same pharmaceutical formulation. In another embodiment, the first and second (and/or third) components of these kits are contained in separate pharmaceutical formulations. Where the first and second (and/or third) components are contained in separate pharmaceutical formulations, the packaged kit may include instructions that include directions for carrying out drug administration of the first and second (and/or third) components sequentially or concurrently.

Representative cAMP elevators or agents that mimic cAMP and additional active compounds from other classes of therapeutic agents for use within the methods, pharmaceutical compositions, and kits of the present invention are described more fully below. Assays and methods for identifying and testing additional cAMP elevators or agents that mimic cAMP for use in the methods and compositions of the invention are well known in the art and include the in vitro and in vivo experimental models described and used throughout the Experimental section below.

cAMP Elevators or Agents that Mimic cAMP for Use in the Methods and Compositions of the Invention

As described above, the present invention relates to methods of use and compositions comprising cAMP elevators or agents that mimic cAMP, alone or in combination with one or more additional active compounds from other classes of therapeutic agents. Suitable cAMP elevators or agents that mimic cAMP include, but are not limited to, the following types of therapeutic agents: an adenylate cyclase activator, a PDE inhibitor, a Toll-like receptor ligand, a calcium activator, a protein kinase A activator, a protein kinase C activator, and adenylate cyclase toxin. Each of these types of cAMP elevators or agents that mimic cAMP are described in more detail below.

### Adenylate Cyclase Activators

Adenylate cyclase is an enzyme that synthesizes cAMP from ATP. There are at least nine isoforms of adenylate cyclase, which differ considerably in regulatory properties and are differentially expressed among tissues (Chen et al. (2000) Science 289:625-628; Houslay & Milligan (1997) Trends Biochem. Sci. 22:217). Early studies indicated that cyclase activity was regulated primarily by interactions with alpha subunits of heterotrimeric G proteins, which are activated through G protein-coupled receptors. More recently, it has become clear that cyclase activity is regulated by multiple effectors, which include not only the alpha subunits of Gₛ and Gᵢ proteins, but also the beta-gamma subunits of G proteins and protein kinase C. Five of the adenylate cyclases known are regulated by calcium (Cooper et al. (1995) Nature 374:421-424; Mons et al. (1998) Life Sciences 62:1647). All known adenylate cyclases are stimulated by exposure of cells to forskolin.

Any compound or agent that enhances adenylate cyclase activity in vivo to elevate intracellular levels of cAMP can be used to practice the present invention. Exemplary adenylate cyclase activators include, but are not limited to, the labdane diterpenes, such as forskolin or a derivative or analog thereof, pyrazole derivatives, benzyloxybenzaldehyde analog, G-protein coupled receptor agonists, and G-protein activators.

Thus, in one embodiment, the cAMP elevator for use within the methods and compositions of the invention is a labdane diterpene such as labdane, forskolin, or a forskolin derivative or analog. The chemical structure for labdane is depicted below:

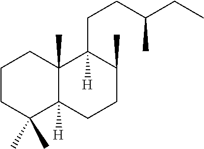

The chemical structure of forskolin is depicted below:

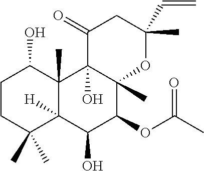

The following numbering system for the forskolin skeletal structure is used throughout the specification and appended claims:

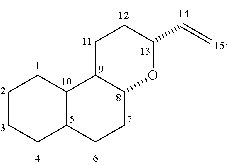

As used herein, a ( [symbol] ) line indicates that the substituent group is projected below the average plane of the six-membered ring to which it is attached and is denoted as alpha (α) in the named compounds, whereas a ( [symbol] ) line indicates that the substituent group is projected above the average plane of the six-membered ring and is denoted as beta (β) in the named compounds. One of ordinary skill in the art would recognize, however, that throughout the specification and claims, a given chemical structure, formula, or name shall encompass all optical and stereoisomers, as well as racemic mixtures where such isomers and mixtures exist.

In some embodiments, the cAMP elevator used in the methods and compositions of the present invention is a labdane diterpine compound according to Formula I, wherein the compound of Formula I has the general structure:

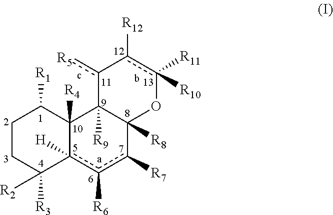

wherein:
a is an optional bond located at the 5,6 or 6,7 positions, and when present at the 5,6 position, the hydrogen atoms at C₅ and C₆ are absent, and when present at the 6,7 position, the hydrogen atoms at C₆ and C₇ are absent;
b is an optional bond located at the 12,13 position, and when present R₁₁ and R₁₂ are absent;
c is an optional bond between C₁₁ and R₅;
R₁ is selected from the group consisting of H, hydroxyl, —OR₁₃, and —O—C(═O)R₁₄, wherein R₁₃ and R₁₄ are each independently alkyl or substituted alkyl;
R₂, R₃, R₄, R₈, and R₁₀ are each independently selected from the group consisting of alkyl, substituted alkyl, aryl, and substituted aryl;
R₅ is O or S, when c is present, and R₅ is —OR₁₅, when c is absent, wherein R₁₅ is selected from the group consisting of H, alkyl, substituted alkyl, C₁-C₆ carboxylic acyl, and trifluoroacetyl;
R₉ is selected from the group consisting of H, hydroxyl, —OR₁₆, and —O—C(═O)R₁₇, wherein R₁₆ and R₁₇ are each independently alkyl or substituted alkyl; or
R₁ and R₉ together form

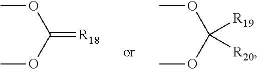

wherein R₁₈ is O or S, and R₁₉ and R₂₀ are each independently selected from the group consisting of H, alkyl, substituted alkyl, alkoxyl, alkenyl, alkynyl, and

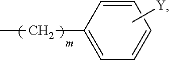

wherein:
   m is an integer from 1 to 8; and
   Y is selected from the group consisting of H, halogen, alkyl, substituted alkyl, alkoxyl, alkylthio, hydroxyl, —CF₃, —NO₂, —CN, phenyl, benzyl, phenoxy, and NR₂₁R₂₂, wherein R₂₁ and R₂₂ are the same or different and are selected from the group consisting of H, alkyl, and substituted alkyl;
R₁₁ is selected from the group consisting of H, alkyl, substituted alkyl, cycloalkyl, substituted cycloalkyl, —CH₂OH, —C(═O)H, —C(═O)OR₂₃, —CH═CR₂₄R₂₅, and —C≡CR₂₆,
wherein:
   R₂₃, is selected from the group consisting of H, alkyl, and substituted alkyl;
   R₂₄ and R₂₅ are each independently selected from the group consisting of H, halogen, —CN, alkyl, substituted alkyl, aryl, substituted aryl, benzyl, substituted benzyl, and —C(═O)(O)ₙR₂₇,
   wherein:
      n is an integer from 0 to 1;
      R₂₇ is selected from the group consisting of H, alkyl, substituted alkyl, aryl, substituted aryl, benzyl, and substituted benzyl;
   R₂₆ is H, alkyl, substituted alkyl, alkoxyl, —CHOH—C≡C—R₂₈, —CH═C=CHR₂₉, —CH═N—OR₃₀, —C(═O)OR₃₁,

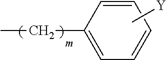

wherein m and Y are as defined above, and

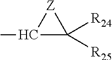

wherein:
   m, Y, Z, R₂₄ and R₂₅ are as defined above,
   R₂₈, R₂₉, R₃₀, and R₃₁ are each independently selected from the group consisting of H, alkyl, substituted alkyl, aryl, substituted aryl, benzyl, and substituted benzyl,
—CH(ZR₃₂)₂,
   wherein:
      Z is as defined above;
      R₃₂ is selected from the group consisting of alkyl, substituted alkyl, aryl, substituted aryl, benzyl, substituted benzyl, or the two groups R₃₂ together form —(CH₂)ₙ—, wherein n is an integer from 2 to 3;

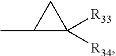

wherein:
   R₃₃ and R₃₄ are each independently selected from the group consisting of H, halogen, alkyl, substituted alkyl, aryl, substituted aryl, benzyl, and —C(═O)(O)ₙR₂₇, wherein n and R₂₇ are as defined above; and
—CH═N—NR₃₅R₃₆,
   wherein R₃₅ and R₃₆ are each independently selected from the group consisting of H, alkyl, substituted alkyl, aryl, substituted aryl, benzyl, substituted benzyl, —COR₃₇, SO₂R₃₈, and C(═O)OR₃₉, wherein R₃₇, R₃₈, and R₃₉ are selected from the group consisting of alkyl, substituted alkyl, aryl, substituted aryl, benzyl, and substituted benzyl;
R₁₂ is selected from the group consisting of H and halogen;
R₆ and R₇ are the same or different and are selected from the group consisting of H, (═O), —OR₄₀, —O—C(═O)—CR₄₁R₄₂(CH₂)ₚR₄₃, —SO₃OR₄₄ and,
wherein R₄₀ is selected from the group consisting of H, alkyl, substituted alkyl, cycloalkyl, substituted cycloalkyl, cycloheteroalkyl, substituted cycloheteroalkyl, C₁-C₆ carboxylic acyl,

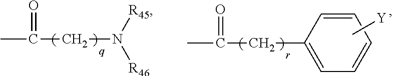

wherein:
   p, q, and r are each independently an integer from 0 to 10;
   Y is defined as above;
   R₄₁ and R₄₂ are each independently selected from the group consisting of H, alkyl, and substituted alkyl;
   R₄₃ is selected from the group consisting of H, halogen, alkyl, substituted alkyl, and NR₄₇R₄₈;
   R₄₄ is selected from the group consisting of H, alkyl, and substituted alkyl;
   R₄₅, R₄₆, R₄₇, and R₄₈ are each independently selected from the group consisting of H, alkyl, substituted alkyl; or
   R₄₅ and R₄₆ or R₄₇ and R₄₈ can be combined to form a 3- to 6-membered cycloalkyl or cycloheteroalkyl ring;
   R₄₀ is selected from the group consisting of H, alkyl, and substituted alkyl;

or R₆ and R₇ together form a carbonate ester of the following formula:

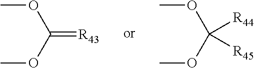

wherein:
   R₄₃ is O or S; and
   R₄₄ and R₄₅ are each independently selected from the group consisting of H, halogen, alkyl, substituted alkyl, aryl, substituted aryl, benzyl, and —C(═O)(O)ₙR₂₇, wherein n and R₂₇ are as defined above;

and pharmaceutically acceptable salts thereof.

In some embodiments, R₁ is —OR₁₃ and R₁₃ is selected from the group consisting of H, —C(═O)C(═O)OH, —C(═O)(CH₂)₂C(═O)OH, —C(═O)(CH₂)₃C(═O)OH, —C(═O)(CH₂)₄C(═O)OH, —C(═O)CH₂NH₂, —C(═O)(CH₂)₃NH₂, —C(═O)(CH₂)₅NH₂, and —C(═O)(CH₂)₃N(CH₃)₂. In some embodiments, R₆ and R₇ are each —OR₄₀ and each R₄₀ is independently selected from the group consisting of H, —C(═O)CH₃, —C(═O)CH₂CH₃, —C(═O)(CH₂)₂CH₃, —C(═O)(CH₂)₂C(═O)OH, —C(═O)(CH₂)₃C(═O)OH, —CH(OH)CH₂OH, —C(═O)CH₂N(CH₃)₂, —C(═O)(CH₂)₂NH₂₅—C(═O)CH₂NHCH₃, —C(═O)CH₂N(CH₃)₂, —C(═O)(CH₂)₂N(CH₃)₂, —C(═O)(CH₂)₃N(CH₃)₂, —C(═O)CH₂N(CH₂CH₃)₂, —C(═O)(CH₂)₂N(CH₂CH₃)₂, —C(═O)CH₂NH(CH₂)₃CH₃,

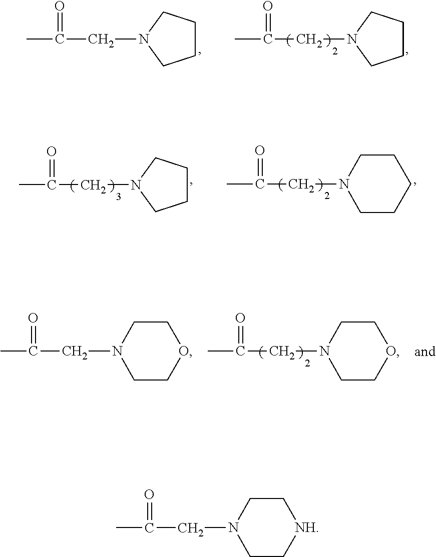

In some embodiments, R₁₁ is selected from the group consisting of —CH₂CH₃, —CH═CH₂, and

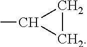

Such embodiments are disclosed, for example, in U.S. Pat. No. 4,954,642 to Tatee et al., which is incorporated herein by reference in its entirety.

In some embodiments, R₆ or R₇ is selected from the group consisting of —OH, —OC(═O)CH₃, —OC(═O)CH₂Cl, —OC(═O)(CH₂)₂Cl, —OC(═O)(CH₂)₃Cl, —OC(═O)NHCH₃, —OC(═O)(CH₂)₂N(CH₃)₂,

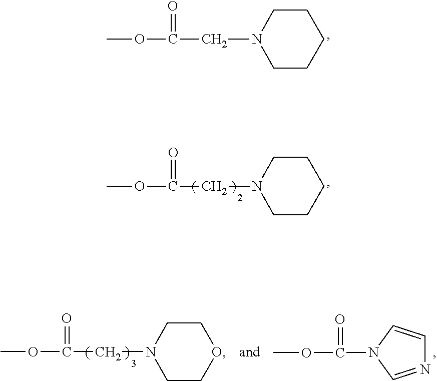

which are disclosed in U.S. Pat. No. 5,268,471 to de Souza et al., which is incorporated herein by reference in its entirety.

In some embodiments, R₁₁ is selected from the group consisting of ethyl, vinyl, e.g., —CH═CH₂, and cyclopropyl.

In some embodiments, R₄₀ is selected from the group consisting of dimethylaminoacetyl, dimethylaminopropionyl, dimethylaminobutyryl, dimethylaminopentanoyl, dimethylaminohexanoyl, aminopropionyl, aminobutyryl, aminopentanoyl, aminohexanoyl, pyrrolidinoacetyl, piperidinopropionyl, and morpholinoacetyl, as disclosed in U.S. Pat. No. 5,789,439 to Hosono et al., which is incorporated herein by reference in its entirety.

In some embodiments, n is 2 and R₂₆ and R₂₇ are each methyl. Such embodiments include 6-(3-dimethylaminopropionyl)forskolin (NKH477). In some embodiments, R₂₆ and R₂₇ together form a cycloheteroalkyl including pyrrolidine, piperidine, and morpholine.

In some embodiments, the compound of Formula I is selected from the group consisting of forskolin, 6-acetyl-7-deacetyl-forskolin, 7-deacetyl-forskolin, 7-deacetyl-6-(N-acetylglycyl)-forskolin, 7-deacetyl-7-O-hemisuccinyl-forskolin, 7-deacetyl-7-[O—(N-methylpiperazino)-γ-butyryl-forskolin, 7-[[2-aminoethyl)amino]carbonyl]-7-desacetylforskolin (7-HPP-forskolin), 6-[[2-aminoethyl)amino]carbonyl]-6-desacetylforskolin (6-HPP-forskolin), and 6-(3-dimethylaminopropionyl)forskolin (NKH477), and pharmaceutically acceptable salts thereof.

In some embodiments, the compound of Formula I is selected from the group consisting of 6-dimethylaminoacetylforskolin, 6-(3-dimethylaminopropionyl)forskolin, 6-(4-dimethylaminobutyryl)forskolin, 7-deacetyl-7-(2,3-dihydroxypropionyl)forskolin, 6-(4-aminobutyryl)forskolin, 6-pyrrolidinoacetyl)forskolin, and 6-(4-dimethylaminobutyryl)-14,15-dihydroforskolin, which are disclosed in U.S. Pat. No. 4,954,642 to Tatee et al., including compounds disclosed in Table 1, col. 9 through col. 14, and the Examples, col. 15 through col. 33, the disclosure of which is incorporated herein by reference in its entirety.

In some embodiments, the compound of Formula I is selected from the group consisting of a 6-acyl-, 7-acyl-, or 6,7-diacyl derivatives of forskolin, including:
8,13-Epoxy-6β,7β-dihydroxy-1α,9α-O-isopropylidene-labd-14-en-11-one;
7β-chloroacetyloxy-8,13-epoxy-6β-hydroxy-1α,9α-O-isopropylidene-labd-14-en-11-one;
7β-(3-chloropropionyloxy)-8,13-epoxy-6β-hydroxy-1α,9α-O-isopropylidene-labd-14-en-11-one;
7β-(4-chlorobutyryloxy)-8,13-epoxy-6β-hydroxy-1α,9α-O-isopropylidene-14-en-11-one;
7β-piperidinoacetyloxy-8,13-epoxy-6β-hydroxy-1α,9α-O-isopropylidene-labd-14-en-11-one;
7β-(3-piperidinopropionyloxy)-8-13-epoxy-6β-hydroxy-1α,9α-O-isopropylidene-labd-14-en-11-one;
7β-(3-dimethylaminopropionyloxy)-8,13-epoxy-6β-hydroxy-1α,9α-O-isopropylidene-labd-14-en-11-one;
7β-(4-morpholinobutyryloxy)-8,13-epoxy-6β-hydroxy-1α,9α-β-isopropylidene-labd-14-en-11-one;
6β-(piperidinoacetyloxy)-8,13-epoxy-7β-hydroxy-1α,9α-O-isopropylidene-labd-14-en-11-one;
6β-(3-piperidinopropionyloxy)-8,13-epoxy-7β-hydroxy-1α,9α-β-isopropylidene-labd-14-en-11-one;
6β-(3-dimethylaminopropionyloxy)-8,13-epoxy-7β-hydroxy-1α,9α-O-Isopropylidene-labd-14-en-11-one;
7β-Acetoxy-6β-piperidinoacetyloxy-8,13-epoxy-1α,9α-O-isopropylidene-labd-14-en-11-one;
7β-Acetoxy-6β-(3-dimethylaminopropionyloxy)-8,13-epoxy-1α,9α-O-isopropylidene-labd-14-en-11-one;
7β-Acetoxy-6β-(3-piperidinopropionyloxy)-8,13-epoxy-1α,9α-O-isopropylidene-labd-14-en-11-one;
8,13-Epoxy-7α-hydroxy-1α,9α-O-isopropylidene-labd-5,14-dien-11-one;
7β-Acetoxy-8,13-epoxy-1α,9α-O-isopropylidene-labd-5,14-dien-1-one;
7β-imidazolylcarbonyloxy-8,13-epoxy-1α,9α-O-isopropylidene-labd-5,14-dien-11-one;
7α-(N-methylaminocarbonyloxy)-8,13-epoxy-1α,9α-O-isopropylidene-labd-5,14-dien-11-one;
6β-piperidinoacetyloxy-1α, 7β,9α-trihydroxy-8,13-epoxy-labd-14-en-11-one;
6β-(3-dimethylaminopropionyloxy)-1α,7β,9α-trihydroxy-8,13-epoxy-labd-14-en-11-one;
7β-acetoxy-6β-(piperidinoacetyloxy)-1α,9α-dihydroxy-8,13-epoxy-labd-14-en-11-one;
7β-acetoxy-6β-(3-dimethylaminopropionyloxy)-1α,9α-dihydroxy-8,13-epoxy-labd-14-en-11-one;
7β-acetoxy-6β-(3-dimethylaminopropionyloxy)-1α,9α-dihydroxy-8,13-epoxy-labd-14-en-11-one hydrochloride;
7β-acetoxy-6β-(3-piperidinopropionyloxy)-1α,9α-dihydroxy-labd-5,14-dien-11-one hydrochloride hemihydrate;
7β-(N-methylaminocarbonyloxy)-8,13-epoxy-1α,9α-dihydroxy-labd-5,14-dien-11-one; which are disclosed in U.S. Pat. No. 5,268,471 to de Souza et al., which is incorporated herein by reference in its entirety.

In other embodiments, the compound of Formula I is selected from the group consisting of 6-(4-dimethylaminobutyryl)forskolin, 6-(5-dimethylaminopentanoyl)-forskolin, 6-(6-dimethylaminohexanoyl)forskolin, 6-(3-aminopropionyl)forskolin, 6-(4-aminobutyryl)forskolin, 6-(5-aminopentanoyl)forskolin, 6-(6-aminohexanoyl)forskolin, 14,15-dihydro-6-(3-dimethylaminopropionyl)forskolin, and 14,15-dihydro-6-(4-dimethylaminobutyryl)forskolin, as disclosed in U.S. Pat. No. 5,789,439 to Hosono et al., which is incorporated herein by reference in its entirety.

In some embodiments, the compound of Formula I is selected from the group consisting of:
13-epoxy-1α,6β,7β-trihydroxy-labd-14-en-11-one 7-acetate;
8,13-epoxy-1α,7β, 9α-trihydroxy-labd-5(6)-ene-11-one 7-acetate;
8,13-epoxy-1α,6β, 7β-trihydroxy-labd-14-en-11-one 6-acetate;
8,13-epoxy-1α,7β,9α-trihydroxy-labd-5(6),14-diene-11-one 7-acetate;
8,13-epoxy-1α,6β, 7β-trihydroxy-labd-14-en-11-one;
8,13-epoxy-1α,6β,7β,11β-tetrahydroxy-labd-14-ene;
8,13-epoxy-1α,6β,7β,11β-tetrahydroxy-labd-14-ene-7-acetate;
12-chloro-8,13-epoxy-1,7β-dihyroxy-labda-5(6),14-dien-11-one 7-acetate;
15-nor-8,13-epoxy-1α,6β,7β,9α-tetrahydroxy-labdan-11-one;
15-nor-8,13-epoxy-1α,6β,7β,9α-tetrahydroxy-labdan-1′-one 7-acetate;
and 8,13-epoxy-1α,7β,9α-trihydroxy-labd-5(6),14-diene 7-propionate, which are disclosed in U.S. Pat. No. 4,517,200 to Kreutner et al., which is incorporated herein by reference in its entirety.

In some embodiments, the compound of Formula I is a 6-(substituted-aminopropionyl) derivative of forskolin including:
7β-Acetoxy-6β-[(3-dimethylaminopropionyl)oxy]-1α,9α-dihydroxy-8,13-epoxy-labd-14-en-11-one hydrochloride;
7β-Acetoxy-60-[(3-piperidinopropionyl)oxy]-1α,9α-dihydroxy-8,13-epoxy-labd-14-en-11-one hydrochloride hemihydrate;
7β-Acetoxy-6β-[(3-N-methylpiperazinopropionyl)oxy]-1α,9α-dihydroxy-8,13-epoxy-labd-14-en-11-one hydrochloride; and
7β-Acetoxy-60-[(3-morpholinopropionyl)oxy]-1α,9α-dihydroxy-8,13-epoxy-labd-14-en-11-one hydrochloride, as disclosed in U.S. Pat. No. 5,869,523 to de Souza et al., which is incorporated herein by reference in its entirety.

In some embodiments, the compound of Formula I is an aminoalkylcarbamyl derivative of forskolin, including 1-aminoalkylcarbamates, 9-aminoalkylcarbamates, 7-aminoalkylcarbamates, 6-aminoalkycarbamates, 6,7-diaminoalkylcarbamates, 1,6-diaminoalkylcarbamates, 1,7-diaminoalkylcarbamates, and 1,6,7-triaminoalkylcarbamates of forskolin, which can be used as intermediates in the synthesis of forskolin derivatives, as disclosed in U.S. Pat. No. 5,350,864 to Seamon et al., which is incorporated herein by reference in its entirety.

In some embodiments, the compound of Formula I is a 12-halogenated forskolin derivative, including 12-chlorodesacetylforskolin, 12-chloroforskolin, 12-bromodesacetylforskolin, 12-bromodesacetylforskolin, 12-fluorodesacetylforskolin, and 12-fluoroforskolin, which are disclosed in U.S. Pat. No. 4,871,764 to Schutske, which is incorporated herein by reference in its entirety.

In some embodiments, the forskolin derivative or analog for use within the methods and compositions of the invention is 6-acetyl-7-deacetyl-forskolin, 7-deacetyl-forskolin, 7-deacetyl-6-(N-acetylglycyl)-forskolin, 7-deacetyl-7-β-hemisuccunyl-forskolin, 7-deacetyl-7-(O-N-methylpiperazino)-γ-butryl-dihydrochlonde-forskolin, 7-HPP-forskolin, 6-HPP-forskolin, or colforsin daropate hydrochloride (NKH477).

Additionally, the labdane diterpenes such as labdane, forskolin, or forskolin derivatives or analogs as described herein can be administered as pharmaceutically acceptable salts. The phrase “pharmaceutically acceptable salt(s),” as used herein, means those salts of the presently disclosed compounds that are safe and effective for use in a subject and that possess the desired biological activity. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, borate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, pamoate (i.e., 1,1′-methylene-bis-(2-hydroxy-3-naphthoate)), mesylate salts. Certain of the presently disclosed compounds can form pharmaceutically or cosmetically acceptable salts with various amino acids. Suitable base salts include, but are not limited to, aluminum, calcium, lithium, magnesium, potassium, sodium, zinc, and diethanolamine salts. For a review on pharmaceutically acceptable salts see Berge et al. (1977) J. Pharm. Sci. 66:1-19, which is incorporated herein by reference. The salts of the compounds described herein can be prepared, for example, by reacting the appropriate equivalent of the compound with the desired acid or base in solution. After the reaction is complete, the salts can be crystallized from solution by the addition of an appropriate amount of solvent in which the salt is insoluble.

Other adenylate cyclase activators for use in the methods and compositions of the invention include, but are not limited to, G-protein coupled receptor agonists and G-protein activators. Adenylate cyclase in mammalian cells is normally activated by the stimulatory regulatory protein Gₛ and guanosine triphosphate (GTP); however, the activation is normally brief because an inhibitory regulatory protein (Gᵢ) hydrolyzes the GTP. Cholera toxin and pertussis toxin catalyze the covalent incorporation of ADP-ribose into the G-protein a-subunit (Nowak and Zawilska (1999) Postepy. Hig. Med. Dosw. 53:147; Sunahara et al. (1996) Annu. Rev. Pharmacol. Toxicol. 36:461; MacNeil et al. (1985) Cell Calcium 6:213; Stiles (1989) J. Cardiovasc. Pharmacol. 14 (Suppl 5):S1). The pertussis toxin A subunit catalyzes the ADP-ribosylation of Gᵢ at a site that impairs the ability of this heterotrimeric G-protein to interact with receptors, thereby blocking the inhibitory effects of Gᵢ on adenylate cyclase. In this manner, the conversion of ATP to cAMP is stimulated. The cholera toxin A subunit catalyzes the attachment of ADP-ribose to Gₛ in a manner that stabilizes the GTP-bound form resulting in persistent activation of adenylate cyclase. Purified subunits of these toxins (e.g., cholera toxin A subunit) have also been shown to activate adenylate cyclase.

Accordingly, suitable G-protein coupled receptor agonists for use in the methods and compositions of the invention include, but are not limited to, a catecholamine, dopamine, dobutamine, isoproterenol, adenosine, carbacyclin, endothelin, epinephrine, glucagon, octopamine, pituitary adenylate cyclase-activating peptide (PACAP), parathyroid hormone, prostaglandin, and vasopressin. Exemplary G-protein activators for use in the methods and compositions of the invention include, but are not limited to, cholera toxin or a subunit thereof and pertussis toxin or a subunit thereof.

Still further adenylate cyclase activators for use in the methods and compositions of the invention include the pyrazole derivative A02011-1 (see Yu et al. (1995) Br. J. Pharmacol. 114:1227-1235) and benzyloxybenzaldehyde and analogs thereof such as those disclosed in Chang et al. (2001) Bioorg. Med. Chem. Lett. 11:1971-1974.

Other cAMP Elevators or Agents that Mimic cAMP

Other compounds for use in the methods and compositions of the present invention include cAMP elevators such as PDE inhibitors, Toll-like receptor ligands, calcium activators, activators of protein kinase C, and adenylate cyclase toxin, and agents that mimic cAMP such as activators of protein kinase A.

Accordingly, in one embodiment, the cAMP elevator for use in the methods and compositions of the present invention is a PDE inhibitor. Cyclic nucleotide phosphodiesterases (PDEs) are enzymes that regulate the cellular levels of the second messengers, cAMP and cGMP, by controlling their rates of degradation. There are 11 different PDE families, with each family having different selectivities for cyclic nucleotide substrates as follows: PDE1 (cAMP/cGMP), PDE2 (cAMP/cGMP), PDE3 (cAMP), PDE4 (cAMP), PDE5 (cGMP), PDE6 (cGMP), PDE7 (cAMP), PDE8 (cAMP), PDE9 (cGMP), PDE10 (cAMP/cGMP), and PDE11 (cAMP/cGMP).

Both nonspecific and selective or partially selective PDE inhibitors are known and may be used within the methods and compositions of the present invention. For example, the non-specific PDE inhibitor, 3-Isobutyl-1-methylxanthine (IBMX), significantly increases intracellular cAMP levels in human bladder epithelial cells compared to untreated controls. Other non-specific PDE inhibitors include, but are not limited to, theophylline, theobromine, aminophylline, pentoxifylline, and caffeine and other methyl xanthine and non-xanthine derivatives.

Selective or partially selective PDE inhibitors for use in the methods and compositions of the invention include, but are not limited to, Vinpocetine (e.g., INTELECTOL®) (available from, e.g., Covex Pharma Inc., Miami, Fla.); Nicardipine HCl (available from, e.g., Par Pharamceutical Companies, Inc., Spring Valley, N.Y.); 8-MeOM-IBMX (8-methoxymethyl-3-isobutyl-1-methylxanthine) (available from Biomol International LP, Plymouth Meeting, Pa.); EHNA (erythro-9-(2-hydroxy-3-nonyl)adenine) (available from, e.g., A.G. Scientific, San Diego, Calif.); IC933 (see, e.g., Snyder et al. (2005) J. Lipid Res. 46:494-503); 2-(3,4-Dimethoxybenzyl)-7-[(1R)-1-[(1R)-1-hydroxyethyl]-4-phenylbutyl]-5-methylimidazo[5,1-f][1,2,4]triazin-4(3H)-one (Bay 60-7550) (available from, e.g., Axxora, LLC, San Diego, Calif.); Lixazinone (available from Syntex Corporation, Palo Alto, Calif.); Cilostamide (available from, e.g., Sigma-Aldrich, Co., St. Louis, Mo.); Milrinone (e.g., PRIMACOR®, discontinued by Sanofi-Aventis, Bridgewater, N.J.) (available from, e.g., Haorui Pharma-Chem, Inc., Edison, N.J.); Cilostazol (available from, e.g., Mylan Pharmaceuticals, Inc., Morgantown, W. Va.); OPC-33540 (6-[3-[3-cyclooctyl-3-[(1R*,2R*)-2-hydroxycyclohexyl]ureido]-propoxy]-2(1H)-quinolinone) (see Sudo et al. (2000) Biochem Pharmacol. 59:347-56); Dihydropyridazinone (for representative derivatives thereof, see U.S. Pat. No. 4,921,856 to Schickaneder et al.); Sildenafil citrate (e.g., VIAGRA®, available from Pfizer, Inc., New York, N.Y.); Zaprinast (available from, e.g., A.G. Scientific, San Diego, Calif.); Dipyridamole (e.g., PERSANTINE®, available from Boehringer Ingelheim Pharmaceuticals, Inc., Ridgefield, Conn.); ARIFLO® (cilomilast) (available from GlaxoSmithKline, Research Triangle Park, N.C.); Vardenafil HCl (LEVITRA®) (available from Schering Corporation, Kenilworth, N.J.); Tadalafil (CIALIS®) (available from Lilly ICOS LLC, Indianapolis, Ind.); E4021 (sodium 1-[6-chloro-4-(3,4-methylenedioxybenzyl)-aminoquinazolin-2-yl]piperidine-4-carboxylate sesquihydrate) (available from Eisai Co., Ltd., Tokyo, Japan); DMPPO (1,3-dimethyl-6-(2-propoxy-5-methanesulfonylamidophenyl)pyrazol[3,4d]-pyrimidin-4-(5H)-one) (available from GlaxoSmithKline, Les Ulis, France); 3-(N,N-dimethylsulfonamido)-4-methyl-nitrobenzene (BRL 50481) Biomol International LP, Plymouth Meeting, Pa.); IC242 (available from Lilly ICOS LLC, Indianapolis, Ind.); BMS-586353 (available from Bristol-Myers Squibb Company, New York, N.Y.); Thiadiazoles; SCH 51866 (cis-5,6a,7,8,9,9a-hexahydro-2-(4-(trill oromethyl)phenylmethyl)-5-methyl-cyelopent(4,5)imidazo[2,1-b)purin-4(3H)-one) (available from Schering-Plough Corporation, Kenilworth, N.J.); and Papaverine (available under several brand names, depending on which salt is desired) (available from, e.g., MP Biomedicals, Inc., Irvine, Calif.). A summary of the selectivity profiles of these compounds for different members of the PDE family is provided in Table 1.

TABLE 1
Selective or Partially-Selective PDE Inhibitors.

| PDE | |
|---|---|
| Family | Compounds |
| PDE1 | Vinpocetine, Nicardipine, 8-MeOM-IBMX |
| PDE2 | EHNA, IC933, Bay 60-7550 |
| PDE3 | Lixazinone, Cilostamide, Milrinone, Cilostazol, OPC-33540, |
| | Dihydropyridazinone |
| PDE4 | Rolipram, Ro 20-1724, Denbufylline, Cilomilast, Roflumilast, |
| | SCH 351591, V11294A, AWD 12-281, L-826,141 |
| PDE5* | Sildenafil, Zaprinast, Dipyridamole, Vardenafil, Tadalafil, |
| | E4021, DMPPO |
| PDE6 | Zaprinast, Dipyridamole, Vardenafil, Tadalafil, E4021, DMPPO |
| PDE7 | Dipyridamole, BRL 50481, IC242, BMS-586353, Thiadiazoles |
| PDE8 | Dipyridamole |
| PDE9 | Zaprinast, SCH 51866 |
| PDE10 | Dipyridamole, Papaverine |
| PDE11 | Tadalafil, Zaprinast, Dipyridamole |

In another embodiment, the PDE inhibitor for use within the methods and compositions of the present invention is a cAMP-specific PDE inhibitor. In one embodiment, the cAMP-specific inhibitor is a PDE3 inhibitor, a PDE4 inhibitor, a PDE7 inhibitor, or a PDE8 inhibitor, including, but not limited to, compounds described above and summarized in Table 1.

In a particular embodiment, the cAMP-specific PDE inhibitor for use within the methods and compositions of the present invention is a PDE4 inhibitor. PDE4 is the predominant cyclic AMP degrading enzyme in most inflammatory and immune cells and exhibits broad anti-inflammatory/immuno-modulatory action (see, e.g., Baumer et al. (2006) Inflammation & Allergy—Drug Targets, 6:17-26). The PDE4 family encompasses four subtypes, which are designated PDE4 A-D and differ in their regulatory behavior and tissue expression patterns. PDE4 inhibitors exhibit structural diversity and include compounds as described above in Table 1, as well as xanthine derivatives, such as arofylline (available from Almirall Prodesfarma, S.A.) and cipamfylline (GlaxoSmithKline, Research Triangle Park, N.C.); catechol derivatives, such as rolipram (EMD Biosciences, San Diego, Calif.), Ro 20-1724 (A.G. Scientific, Inc., San Diego, Calif.), piclamilast, cilomilast (ARIFLO®, GlaxoSmithKline, Research Triangle Park, N.C.), roflumilast (Altana Pharma, Germany), and atizoram; indole derivatives, such as AWD 12-281 (Elbion AG, Germany); and thalidomide derivatives, such as CC-10004 (Celgene Corporation, Summit, N.J.). Such PDE4 inhibitors are described, for example, in Baumer et al. (2006) Inflammation & Allergy—Drug Targets, 6:17-26, which is incorporated herein by reference in its entirety.

Still further PDE4 inhibitors that may be used within the methods and compositions of the invention include CC-10015 (available from Celgene Corporation), 4AZA-PDE41 (available from Elbion NV), ELB353 (available from Elbion NV), ELB326 (available from Elbion NV), GRC 4039 (available from Glenmark Pharmaceuticals Limited), GRC 4039 (available from Glenmark Pharmaceuticals Limited), IPL4088 (available from Inflazyme Pharmaceuticals Ltd.), MEM 1917 (available from Memory Pharmaceuticals Corp), PLX369/PDE 4 Inhibitor (available from Plexxikon Inc), AVE8112 (available from Sanofi-Aventis), Theophylline (available from SCOLR Pharma Inc), Oglemilast (available from Teijin Pharma Limited), Oglemilast/GRC 3886 (available from Teijin Pharma Limited), Z15370A (available from Zambon Group), LAS 37779 (available from Almirall Prodesfarma, S.A.), Atopik (available from Barrier Therapeutics Inc), CC-11050 (available from Celgene Corporation), 256066 (available from GlaxoSmithKline plc), NIK-616 (available from Kowa Co., Ltd.), MEM 1414 (available from Memory Pharmaceuticals Corp), AWD 12-281/GW842470 (available from Elbion NV), Oglemilast (available from Forest Laboratories Inc), 256066 (available from GlaxoSmithKline plc), GW842470/AWD 12-281 (available from GlaxoSmithKline plc), Oglemilast (available from Glenmark Pharmaceuticals Limited), IPL455,903/HT-0712 (available from Helicon Therapeutics, Inc), IPL455,903/HT-0712 (available from Inflazyme Pharmaceuticals Ltd.), MN-166 (ibudilast) (available from MediciNova Inc), OPC-6535 (available from Otsuka America Pharmaceutical, Inc.), Tofimilast (available from Pfizer Inc), Daxas (roflumilast)/APTA-2217 (available from Nycomed), OPC-6535 (available from Otsuka America Pharmaceutical, Inc.), Daxas (roflumilast)/APTA-2217 (available from Tanabe Seiyaku Co., Ltd.), Theolair (theophylline) (available from 3M Company), Dot (drotaverine hydrochloride) (available from Acme Laboratories Ltd.), Thenglate (theophylline) (available from Acme Laboratories Ltd.), Pulmophyllin (theophylline) (available from Adcock Ingram Limited), Solphyllex (theophylline, etofulline, diphenylpyraline hydrochloride, ammonium chloride and sodium citrate) (available from Adcock Ingram Limited), Solphyllin (theophylline and etofylline) (available from Adcock Ingram Limited), Baladex (theophylline, guaifenesin) (available from AFLOFARM), Taverine (drotaverine) (available from Ajanta Pharma Limited), Etafin (acepifylline) (available from Aleppo Pharmaceutical Industries, L.L.C.), Theo-dur (theophylline) (available from Almirall Prodesfarma, S.A.), No-spa (drotaverine hydrochloride) (available from Ambee Pharmaceuticals Ltd.), Contine (theophylline) (available from Aristopharma Ltd.), Etophylline plus Theophylline (available from Arvind Remedies Ltd), Bitophyllin (theophylline and guaifenesin) (available from BARAKAT Pharmaceutical Industries), Theophylline (available from Barr Pharmaceuticals Inc), Theolin (theophylline anhydrous) (available from Beacons Pharmaceuticals Pte Ltd), Theophylline (theophylline anhydrous) (available from Beacons Pharmaceuticals Pte Ltd), Dyphylin Injection (etophylline and theophylline) (available from BELCO Pharma.), Theospirex (theophylline anhydrous) (available from BIOFARM Sp. z o.o.), Asima (doxofylline) (available from Bukwang Pharmaceutical Company Limited), Theobid Tablets (theophylline) (available from Cipla Ltd.), Theoday Tablets (theophylline) (available from Cipla Ltd.), Theoped Syrup (theophylline) (available from Cipla Ltd.), Bronchipret (theophyline) (available from Darya-Varia), Frivent (theophylline) (available from Dompe S.p.A.), Trospa (available from Dr Reddys Laboratories Ltd), Theo-Dur (theophylline) (available from Elan Corp Plc), Dotarin (drotaverine hydrochloride) (available from Elder), Gulamyl (theophylline and guaiphenesin) (available from ELPEN Pharmaceutical Co. Inc.), Drotaverine hydrochloride (available from ELS aad Pharmaceutical Industries), Theophylline (available from Eurand), Puroxan (doxofylline) (available from Eurodrug Laboratories), Choledyl (choline theophyllinate, theophylline) (available from Galenica s.a.), Drotaverine-Grindeks (drotaverine) (available from Grindeks), Tromphylin (theophylline) (available from Grupo Ferrer), Hesotanol (etophylline nicotinate) (available from HanAll Pharmaceutical), Neophin (diethylaminoethyl theophylline) (available from HanAll Pharmaceutical), Arutopa (acepifylline) (available from Hawon Pharmaceutical Corporation), Theophylline (available from Indchemie Health Specialities Pvt. Ltd), Theophylline and Etophylline (available from Indchemie Health Specialities Pvt. Ltd), Doverin (drotaverine) (available from Intas Pharmaceuticals Ltd.), Euphyllinum-N (theophylline) (available from JSC Farmak International), Theophar (theophylline) (available from Julphar), Draw (drotaverine) (available from Kamron Laboratories Ltd.), Quibron-T (theophylline) (available from King Pharmaceuticals Inc), Quibron-T/SR (theophylline anhydrous) (available from King Pharmaceuticals Inc), Theodur (theophylline)/Theodrip (available from Kowa Co., Ltd.), Teotard (theophylline) (available from Krka, d. d.), Theolan-B SR (theophylline) (available from KunWha Pharmaceutical Co., Ltd.), Hespil (acepifylline) (available from Kyung Dong Pharma. Co., Ltd.), Theophylline monohydrate (available from Laboratoires SMB SA), Sedacris (theophylline, guaifenesin) (available from Laboratorio Elea SACIFYA), Aminofilin (theophyllin) (available from Laboratorios Phoenix), Dexa aminofilin (dexamethasone and theophylline) (available from Laboratorios Phoenix), Dexa teosona (dexamethasone and theophylline) (available from Laboratorios Phoenix), Inastmol (ketotifen and theophylline) (available from Laboratorios Phoenix), Teosona (theophylline) (available from Laboratorios Phoenix), Theodur (theophylline) (available from Lavipharm Group), Spacovin Injection (drotaverine) (available from M.J. Group), Drotikind (drotaverine hydrochloride) (available from Mankind Pharma Ltd.), Ranispas-DV (drotaverine, omeprazole hydrocholoride) (available from Mankind Pharma Ltd.), Drot (drotaverine hydrochloride) (available from Mapra Laboratories Pvt. Ltd.), Theodur (theophylline) (available from Mitsubishi Pharma Corporation), Uniphyllin continus (theophylline) (available from Mundipharma International Limited), Unicon/Uniphyl (theophylline) (available from Mundipharma K.K.), Uniphyllin Continus (theophylline) (available from Napp Pharmaceuticals Limited), Theonat (theophylline) (available from Natco Pharma Limited), Theophylline (available from Natco Pharma Limited), Xtma (theophylline and etophylline) (available from Neon Laboratories Ltd.), Unicon (theophylline) (available from Nichi-iko Pharmaceutical Co., Ltd (formerly Nihon Iyakuhin Kogyo Co., Ltd)), Teokap SR (theophylline) (available from Nobel Ilac Sanayii ye Ticaret A.S.), Compound theophylline (available from North China Pharmaceutical Group Corp), Euphyllin/Euphylong (theophylline) (available from Nycomed), Orophil (etofyllin, theophyllin) (available from Ortin Laboratories Limited), Teosona SOL (theophylline) (available from Osmotica Pharmaceutical Corp), Synaclyn (theophylline) (available from Otsuka Pharmaceutical Co., Ltd.), Uniphyl (theophylline) (available from Otsuka Pharmaceutical Co., Ltd.), Choledyl SA (oxtriphylline) (available from Pfizer Inc), Farcophylline (piperazine theophylline ethanoate) (available from Pharco Pharmaceuticals Inc.), Farcosolvin (ambroxol hydrochloric acid, guaiphenesin and theophylline anhydrous) (available from Pharco Pharmaceuticals Inc.), Remind (hexobendine dihydrochloride, etofylline and ethamivan) (available from Pharco Pharmaceuticals Inc.), Theofar S.R (anhydrous theophylline) (available from Pharco Pharmaceuticals Inc.), Pharmaniaga theophylline (theophylline) (available from Pharmaniaga Berhad), pms-Oxytriphylline (oxytriphylline) (available from Pharmascience Inc.), Retaphyl (theophylline) (available from PT Kimia Farma Tbk), T-phyl (theophylline) (available from Purdue Pharma L.P), Uniphyl (theophylline) (available from Purdue Pharma L.P), Teofilina (theophylline) (available from Ranbaxy Laboratories Ltd.), Theostan-CR (theophyline) (available from Ranbaxy Laboratories Ltd.), Theo-dur (theophylline) (available from Recordati SpA), Glyphillin (theophylline sodium glycinate) (available from Rekah Pharmaceutical Industry Ltd.), No Spa (drotaverine) (available from Sanofi-Aventis), Relispa (drotaverine hydrochloride) (available from Searle Pakistan Pvt. LTD.), Respro SR (theophylline) (available from Searle Pakistan Pvt. LTD.), Theotard (theophylline) (available from Sopharma JSCo.), Asmanyl SR (theophylline) (available from Square Pharmaceuticals Ltd.), Espa (drotaverine hydrochloride) (available from Square Pharmaceuticals Ltd.), Broncolin (guaiacol and theophylline) (available from Standard Chem. & Pharm.), TR Phyllin (theophylline) (available from Sun Pharmaceutical Industries Ltd.), Theophylline (available from Themis Laboratories Private Ltd), Teofurmate L (theophylline) (available from Towa Pharmaceutical Co., Ltd.), Teofurmate Dry Syrup (theophylline) (available from Towa Pharmaceutical Co., Ltd.), Theophylline (available from United Research Laboratories and Mutual Pharmaceutical Company), E.T.phyllin (etophylline, theophylline) (available from Vanguard Therapeutics), Vero-Drotaverine (drotaverine) (available from Veropharm), Lungfyl SR Tablet (available from Yash Pharma Laboratories Ltd.), Deoprin retard (theophylline) (available from Yooyoung Pharmaceutical Co., Ltd.), Soluphin (diethylaminoethyl theophylline hydrochloride) (available from YOOYOUNG Pharmaceutical Co., Ltd.), Green (guaiacol glyceryl ether, theophylline sodium glycinate) (available from Yung shin Pharmaceutical), Sentin (diprophylline) (available from Yung shin Pharmaceutical), Spophyllin retard (theophylline) (available from Zentiva, a.s. (formerly Leciva a.s.)), Theolate Liquid (theophylline and guaifenesin) (available from Alpharma Inc), Theophylline Elixir (theophylline) (available from Alpharma Inc), IC485 (available from Array BioPharma Inc), Lirimilast (available from Bayer Ag), Mesopram (available from Bayer Schering Pharma AG), CC 7085 (available from Celgene Corporation), CC-10004 (available from Celgene Corporation), CC-1088 (available from Celgene Corporation), CC-1088 (available from Celgene Corporation), CDC-998 (available from Celgene Corporation), AWD 12-281/GW842470 (available from Elbion NV), IC485 (available from Eli Lilly & Co), Ariflo (available from GlaxoSmithKline plc), GW842470/AWD 12-281 (available from GlaxoSmithKline plc), GRC 3015 (available from Glenmark Pharmaceuticals Limited), GRC 3566 (available from Glenmark Pharmaceuticals Limited), GRC 3590 (available from Glenmark Pharmaceuticals Limited), GRC-3785 (available from Glenmark Pharmaceuticals Limited), KW-4490 (available from Kyowa Hakko Kogyo Co., Ltd.), MEM 1414 (available from Memory Pharmaceuticals Corp), CDP 840 (available from Merck & Co Inc), (MRK)ND1251 (available from Neuro3d), ONO-6126 (available from Ono Pharmaceutical Co., Ltd.), Daxas (roflumilast) (available from Pfizer Inc), MEM 1414 (available from Roche Holdings Ltd), MEM 1917 (available from Roche Holdings Ltd), CDP-840 (available from UCB S.A.), CT-5357 (available from UCB S.A.), ONO-6126 (available from Ono Pharmaceutical Co., Ltd.), and ONO-6126 (available from Santen Pharmaceutical Co., Ltd.).

In another embodiment, the cAMP elevator for use in the methods and compositions of the present invention is a Toll-like receptor ligand. Toll-like receptors are a class of single membrane-spanning non-catalytic receptors that recognize structurally conserved molecules derived from microbes once they have breached physical barriers such as the skin or urinary tract mucosa and activate immune cell responses. The Toll-like receptor family has been described as type I transmembrane pattern recognition receptors that possess varying numbers of extracellular N-terminal leucine-rich repeat motifs, followed by a cysteine-rich region, a TM domain, and an intracellular Toll/IL-1 R (TIR) motif (Hashimoto et al. (1988) Cell 52:269; Medzhitov et al. (1997) Nature 388:394; Rock et al. (1998) Proc. Natl. Acad. Sci. USA 95:588; Chaudhary et al. (1998) Blood 91:4020; Takeuchi et al. (1999) Gene 231:59; Chuang and Ulevitch (2000) Eur. Cytokine Netw. 11:372; Du et al. (2000) Eur. Cytokine Netw. 11:362). The leucine-rich repeat domain is important for ligand binding and associated signaling and the TIR domain is important in protein-protein interactions and is typically associated with innate immunity (Modlin (2002) Ann. Allergy Asthma Immunol. 88:543; Kobe & Deisenhofer (1995) Curr. Opin. Struct. Biol. 5:409; Aravind et al. (2001) Science 291:1279; Dunne and O'Neill (2003) Sci. STKE 2003:re3). The human TLR family is composed of at least 10 members, each of which is specific in its expression patterns and pathogen-associated molecular pattern sensitivities (Chuang & Ulevitch (2001) Biochim. Biophys. Acta 1518:157; Akira (2003) Curr. Opin. Immunol. 15:5-11).

Toll-like receptor ligands that activate the TLR pathway thus represent other cAMP elevators useful in the present invention. Exemplary Toll-like receptor ligands for use within the methods and compositions of the invention include, but are not limited to, lipopolysaccharide (LPS), 1-palmitoyl-2-linoleoyl-sn-glycero-3-phosphocholine (pLPC), lipoteichoic acid (LTA), flagellin, ANA773 (available from Anadys Pharmaceuticals Inc), ANA975 (available from Anadys Pharmaceuticals Inc), AVE0675 (available from Coley Pharmaceutical Group Inc), Vaxlmmune Vaccine Adjuvant (available from Coley Pharmaceutical Group Inc), TLR-9 Agonist (available from Dynavax Technologies Corp), HYB2093/TLR9Agonist (available from Idera Pharmaceuticals Inc), IMO-2125 (available from Idera Pharmaceuticals Inc), IMOxine/HYB2055/IMO-2055 plus Monoclonal antibodies (available from Idera Pharmaceuticals Inc), TLR 7, 8 agonist (available from Idera Pharmaceuticals Inc), TLR 7/8 agonist (available from Idera Pharmaceuticals Inc), TLR7, 8, 9 agonists (available from Idera Pharmaceuticals Inc), IPH 31XX (available from Innate Pharma S.A.), ANA975 (available from Novartis AG), Vaxlmmune Vaccine Adjuvant (available from Novartis AG), HspE7 with Poly-ICLC (available from Nventa Biopharmaceuticals Corp), HspE7 with Poly-ICLC (available from Roche Holdings Ltd), AVE0675 (available from Sanofi-Aventis), SAR 21609 (available from Sanofi-Aventis), Vaxlmmune with BioThrax (available from Coley Pharmaceutical Group Inc), TLR9Agonist with Chemotherapy (available from Dynavax Technologies Corp), 852A (available from 3M Company), IMOxine/HYB2055/IMO-2055 (available from Idera Pharmaceuticals Inc), IMOxine/HYB2055/IMO-2055 with Chemotherapy (available from Idera Pharmaceuticals Inc), Ragweed SC/Pollinex Quattro Ragweed/Pollinex R (available from Allergy Therapeutics plc), PF-3512676/CpG 7909 with Chemotherapy (available from Coley Pharmaceutical Group Inc), Heplisav (available from Dynavax Technologies Corp), Tolamba (available from Dynavax Technologies Corp), E5564 (eritoran) (available from Eisai Co Ltd), Eritoran (available from Eisai Inc.), PF-3512676/CpG 7909 with Chemotherapy (available from Pfizer Inc), TAK-242 (available from Takeda Pharmaceutical Company Limited), Pollinex Quattro Vaccine (available from Allergy Therapeutics plc), Pollinex Quattro Vaccine (available from AllerPharma), E6020 (available from Eisai Co Ltd), Imiquad Cream (imiquimod) (available from Glenmark Pharmaceuticals Limited), Aldara (imiquimod) Cream (available from Graceway Pharmaceuticals, LLC), Imiquimod cream (available from Henan Topfond Pharmaceutical Co., Ltd.), Aldara (available from LAVIPHARM Group), Aldara/MTD-39 (imiquimod) (available from Mochida Pharmaceutical Co., Ltd.), E6020 (available from Sanofi-Aventis), Aldara Cream (available from 3M Company), Resiquimod (available from 3M Company), ANA971 (available from Anadys Pharmaceuticals Inc), Isatoribine/ANA245 (available from Anadys Pharmaceuticals Inc), Actilon/CPG 10101 (available from Coley Pharmaceutical Group Inc), AVE 7279/CpG TLR9Agonists (available from Coley Pharmaceutical Group Inc), CpG 7909 with Engerix-B Vaccine (available from Coley Pharmaceutical Group Inc), PF-3512676/CPG 7909 (available from Coley Pharmaceutical Group Inc), E5531 (available from Eisai Co Ltd), E5564 (eritoran) (available from Eisai Co Ltd), Resiquimod (available from Eli Lilly & Co), CRX-527 (available from GlaxoSmithKline plc), CRX-675 (available from GlaxoSmithKline plc), PF-3512676/CPG 7909 (available from Pfizer Inc), AVE 7279/CpG TLR9Agonists (available from Sanofi-Aventis), AN 033-1 (available from Anadys Pharmaceuticals Inc), TLR-9 Agonist (available from AstraZeneca Plc), and IRS inhibitors (available from Dynavax Technologies Corp).

In some embodiments, the cAMP elevator for use within the methods and compositions of the invention is a calcium channel activator or calcium activator. Calcium channel activators are agents that increase calcium influx into calcium channels and include, but are not limited to, BAY-K-8644 (available from Biomol International), FPL 64176 (available from Biomol International), and Maitotoxin (available from Wako Bioproducts). Calcium activators are agents that increase intracellular calcium release and include, but are not limited to, calcium ionophores and phospholipase C activators. Suitable calcium ionophores for use in the methods and compositions of the present invention include ionomycin calcium salts (available from Sigma) or A23187 (available from Sigma) (see also, Moore et al. (1991) Immunopharmacology 21:1-12; Miyake et al. (1999) J. Urol. 162:916-921). Phospholipase C hydrolyzes phosphatidylinositol bisphosphate (PIP₂) into inositol-1,4,5-triphosphates which mediate intracellular calcium release (Noh et al. (1995) Biochim. Biophys. Acta 1242:99-113; Rhee (2001) Annu. Rev. Biochem. 70:281-312). Suitable phospholipase C activators for use in the methods and compositions of the present invention include 2,4,6-Trimethyl-N-(m-3-trifluoromethylphenyl)benzenesulfonamide (m-3M3FBS; available from Calbiochem)(see also, Bae et al. (2003) Mol. Pharmacol. 63:1043-1050).

Another suitable cAMP elevator for use in the methods and compositions of the present invention is an activator of protein kinase C (PKC). Protein kinase C is a ubiquitous phospholipid-dependent enzyme that is involved in signal transduction associated with cell proliferation, differentiation, and apoptosis. At least eleven closely related PKC isozymes have been reported that differ in their structure, biochemical properties, tissue distribution, subcellular localization, and substrate specificity (Black (2000) Front. Biosci. 5:406; Cooper et al. (1999) Arch. Biochem. Biophys. 372:69; Yamamoto et al. (1998) Exp. Cell Res. 240:349. Rasmussen et al. (1995) Endocr. Rev. 16:649; Taylor et al. (1995) FASEB J. 9:1255; Nishizuka (1995) FASEB. J. 9:484; Newton (1995) J. Biol. Chem. 270:28495; Hanks and Hunter (1995) FASEB J. 9:576). They are classified as conventional, novel, and atypical isozymes. Conventional PKC isozymes are Ca²⁺-dependent, while novel and atypical isozymes do not require Ca²⁺ for their activation. All but the atypical PKC isozymes are activated by diacylglycerol (DAG). Membrane receptor binding of a hormone or other effector molecule results in activation of phospholipase C (PLC) or phospholipase A₂ (PLA₂) via a G-protein-dependent phenomenon. The activated PLC hydrolyzes phosphatidylinositol-4,5-bisphosphate (PIP₂) to produce DAG and inositol-1,4,5-trisphosphate (IP₃). The IP₃ causes the release of endogenous Ca²⁺ that binds to the cytosolic PKC and exposes the phospholipid-binding site. The binding of Ca²⁺ translocates PKC to the membrane, where it interacts with DAG and is transformed into a fully active enzyme.

In particular, PKC activators potentiate forskolin-induced cAMP formation. In some embodiments, the PKC activator for use within the methods and compositions of the invention is phorbol myristate acetate (PMA) or a PKC purified enzyme.

Adenylate cyclase toxin represents another type of cAMP elevator for use in the methods and compositions of the present invention. Adenylate cyclase toxin is a single polypeptide A/B-type bacterial toxin that has the ability to interact with target cells, insert into the cytoplasmic membrane, and deliver its adenylate cyclase enzymatic domain to the cell interior (Mock et al. (1993) Trends in Microbiol. 1:187-192; Hewlett & Maloney (1994) in Handbook of Natural Toxins, Volume 8: Microbial Toxins (Iglewski et al. eds) pp. 425-439, Marcel Dekker, New York). Once entry has occurred, the enzymatic activity of the toxin produces cAMP from host cell ATP (Wolff et al. (1980) Proc. Natl. Acad. Sci. U.S.A. 77:3841-3844). Accordingly, a further cAMP elevator that can be used in the methods and compositions of the present invention is adenylate cyclase toxin.

In another embodiment, an agent that mimics cAMP may be used within the methods and compositions of the invention. Suitable agents that mimic cAMP include protein kinase A (PKA) activators. PKA is normally inactive as a tetrameric holoenzyme, consisting of two catalytic and two regulatory units (Taylor (1989) J. Biol. Chem. 264:8443-8446; Taylor et al. (1990) Annu. Rev. Biochem. 59:971-1005). cAMP binds to the regulatory units of the protein kinase, causing dissociation between the regulatory and catalytic subunits, which in turn activates the catalytic units and enables them to phosphorylate substrate proteins. Suitable PKA activators for use within the methods and compositions of the invention include, but are not limited to, a PKA subunit, cAMP, and cAMP analogs such as 6-Bnz-cAMP, 8-CPT-2′-O-Me-cAMP, 8-CPT-cAMP, 8-Bromo-cAMP, Dibutyryl-cAMP, Dioctanoyl-cAMP, Sp-8-Br-cAMPS, and Sp-cAMPS.

### Additional Active Compounds for Use in the Methods and Compositions of the Invention

The cAMP elevators or agents that mimic cAMP described herein can be used in combination with additional active compounds from other classes of therapeutic agents to further enhance the efficacy of the cAMP elevator or agent that mimics cAMP in treatment of a UTI in a subject. These additional active compounds can be any other therapeutic agent that finds use in treating a UTI, including but not limited to antimicrobial agents such as antibiotics and drugs that block adherence of bacteria to the bladder wall. These additional active compounds can also include cholesterol lowering drugs. Each of these types of additional active compounds is described in more detail below.

### UTI Therapeutic Agents

In some embodiments, the additional active compound is any therapeutic agent in use or in development for treating a UTI. Such agents include antimicrobial agents, which include but are not limited to antibiotics, and drugs that block adherence of bacteria to the bladder wall.

In some embodiments, the antimicrobial agent for use with the presently disclosed methods and compositions is a quinolone antibiotic. Representative quinolone antibiotics include first generation quinolones, including, but not limited to, Cinoxacin (ClNOXACIN®), including Cinobac (available from Watson Pharmaceuticals Inc); Flumequine (FLUMEQUINE® (veterinary use); Nalidixic acid, including, Naligram (nalidixic acid) (available from Acme Laboratories Ltd.), Winlomylon (nalidixic acid) (available from Adcock Ingram Limited), Nalidixic acid (available from Arvind Remedies Ltd), Diarlop (nalidixic acid) (available from Jagsonpal Pharmaceuticals Ltd.), Gramoneg (nalidixic acid) (available from Ranbaxy Laboratories Ltd.), Uriben (nalidixic acid) (available from Rosemont Pharmaceuticals Ltd), Negram (nalidixic acid) (available from Sanofi-Aventis), Negram (nalidixic acid) (available from Searle Pakistan Pvt. LTD.), Nalidixic acid (available from Sterling Lab), and Nalixid (nalidixic acid) (available from Zentiva, a.s. (formerly Leciva a.s.)); oxolinic acid; piromidic acid; and pipemidic acid, including Pipemidic acid (available from AXM Pharma Inc), and Urotractin (pipemidic acid) (available from Eurodrug Laboratories), Urotractin (pipemidic acid) (available from Sanbe Farma); and the like.

Second generation quinolones, include, but are not limited to: ciprofloxacin, including Ciprofloxacin (available from Advancis Pharmaceutical Corp), Ciprofloxacin (available from Aurobind( ) Pharma Ltd.), Ciprofloxacin Hydrochloride Tablets (available from Barr Pharmaceuticals Inc), Cipro XR (ciprofloxacin hydrochloride) (available from Bayer Ag), Cipro I.V. (ciprofloxacin) (available from Biofarma Pharmaceutical Industry Co. Inc.), Proquin XR (ciprofloxacin hydrochloride) (available from Depomed Inc), Proquin XR (ciprofloxacin hydrochloride) (available from Esprit Pharma, Inc.), Ciprofloxacin Extended-release (ER) (available from Mylan Laboratories Inc), Ciprofloxacino (ciprofloxacin) (available from ProStrakan Group plc), and Piprol (ciprofloxacin) (available from ProStrakan Group plc); enoxacin (ENROXIL®, PENETREX®); fleroxacin (MEGALONE® (withdrawn)); lomefloxacin (MAXAQUIN®); nadifloxacin; norfloxacin, including H Norfloxacin (norfloxacin) (available from AC HELCOR Group), Norfloxacin (available from Arvind Remedies Ltd), Norfloxacin (available from BAL PHARMA LTD.), Norspan (norfloxacin) (available from Blue Cross Laboratories, Ltd.), Norflox Tablets (norfloxacin) (available from Cipla Ltd.), Uriflox (norfloxacin) (available from ELSaad Pharmaceutical Industries), Norfloxacin (available from MISSION PHARMACEUTICALS LTD.), Olfron (norfloxacin) (available from Neon Laboratories Ltd.), Duonor (norfloxacin) (available from RPG Life Sciences Ltd.), Norfloxacin (available from Sandoz International GmbH), Shalflox tablets (norfloxacin) (available from Shalina Laboratories Pvt Ltd.), Norfloxacin (available from Shanghai Sunve Pharmaceutical Co., Ltd.), Norfloxacin (available from Sterling Lab), and Zyflox (norfloxacin) (available from Zydus Cadila Healthcare Limited); ofloxacin, such as Ofexin (ofloxacin) (available from Daewon Pharm. Co., Ltd.); pefloxacin; rufloxacin; and the like.

Third generation quinolones, include, but are not limited to, Balofloxacin, including Q-roxin (balofloxacin) (available from Choongwae Pharma Corporation); Grepafloxacin (RAXAR® (withdrawn)); Levofloxacin, including Levaquin (levofloxacin) (available from Ortho-McNeil, Inc.); Pazufloxacin mesilate; Sparfloxacin (ZAGAM®); Temafloxacin (OMNIFLOX® (withdrawn)); tosufloxacin, and the like.

Fourth generation quinolones, include, but are not limited to, clinafloxacin, gatifloxacin, such as Gatiquin Tablets (gatifloxacin) (available from Cipla Ltd.), gemifloxacin (FACTIVE®), moxifloxacin (AVELOX®); sitafloxacin; trovafloxacin; and the like.

Other quinolones include prulifloxacin Sword 100 (prulifloxacin) (available from Meiji Seika Kaisha, Ltd.). Further, ABT-492/WQ-3034 (available from Abbott Laboratories), is a novel fluoroquinolone antibacterial agent that effectively targets DNA gyrase and topoisomerase IV. ABT-492 is less active against human topoisomerase II than the bacterial enzymes, indicating high selectivity for the bacterial enzymes. ABT 492 currently is being developed for the treatment of urinary tract infections.

In other embodiments, the antimicrobial agent for use with the presently disclosed methods and compositions is a cephalosporin antibiotic. Representative cephalosporin antibiotics include first generation cephalosporins, including but not limited to, Cefacetrile (cephacetrile); Cefadroxil (cefadroxyl; DURICEF®), including DURACEF (cefadroxil) (available from Juste S.A.Q.F.), Cefalexin (cephalexin; KEFLEX®); Cephaloglycin; Cefalonium (cephalonium); Cefaloridine (cephaloradine); Cefalotin (cephalothin; KEFLIN®); Cefapirin (cephapirin; CEFADRYL®); Cefatrizine; Cefazaflur; Cefazedone; Cefazolin (cephazolin; ANCEF®, KEFZOL®); Cefradine (cephradine; VELOSEF®); Cefroxadine; Ceftezole; and the like.

Second generation cephalosporins, include, but are not limited to, Cefonicid (MONOCID®); Cefprozil (cefproxil; CEFZIL®); Cefuroxime (ZINNAT®, ZINACEF®, CEFTIN®, BIOFUROKSYM®), including Cefuroxime (available from Advancis Pharmaceutical Corp); Cefuzonam; Cefaclor (CECLOR®, DISTACLOR®, KEFLOR®, RANICLOR®); Cefamandole; Ceforanide; Cefotiam; Carbacephems: loracarbef (LORABID®); Cephamycins: cefbuperazone, cefinetazole (ZEFAZONE®), cefminox, cefotetan (CEFOTAN®), cefoxitin (MEFOXIN®); and the like.

Third generation cephalosporins include, but are not limited to, Cefcapene; Cefdaloxime; Cefdinir (OMNICEF®); Cefditoren; Cefetamet; Cefixime (SUPRAX®); Cefinenoxime; Cefodizime; Cefoperazone (CEFOBID®)Cefotaxime (CLAFORAN®); Cefpimizole; Cefpodoxime (VANTIN®), including Cefoprox (cefpodoxime proxetil) (available from Cipla Ltd.); Cefteram; Ceftibuten (CEDAX®); Ceftiofur; Ceftiolene; Ceftizoxime (CEFIZAX®); Ceftriaxone (ROCEPHIN®); Ceftazidime (FORTUM®, FORTAZ®); Cefpiramide; Cefsulodin; and the like.

Fourth generation cephalosporins include, but are not limited to, Cefclidine; Cefepime (MAXIPIME®); Cefluprenam; Cefoselis; Cefozopran; Cefpirome; Cefquinome; and the like.

Yet to be classified cephalosporins include Cefaclomezine; Cefaloram; Cefaparole; Cefcanel; Cefedrolor; Cefempidone; Cefetrizole; Cefivitril; Cefmatilen; Cefinepidium; Cefovecin; Cefoxazole; Cefrotil; Cefsumide; Ceftioxide; Ceftobiprole (previously BAL 9141 and RO 63-9141); Ceftobiprole (previously BAL 5788); Cefuracetime; and the like.

Further, Doripenem for Injection/S-4661 (available from Johnson & Johnson) and Doripenem for Injection/S-4661 (available from Ortho-McNeil, Inc.), is a member of the carbapenem class of beta-lactam antibiotics, having broad-spectrum bactericidal activity acts by targeting penicillin-binding proteins (PBPs) to inhibit the biosynthesis of the bacterial cell wall.

In yet other embodiments, the antimicrobial agent for use with the presently disclosed methods and compositions is a tetracycline antibiotic including, but not limited to, Chlortetracycline; Oxytetracycline; Tetracycline; Demethylchlortetracycline; Rolitetracycline; Limecycline; Clomocycline; Methacycline; Doxycycline; Minocycline; Tertiary-butylgylcylamidominocylcine; and the like (see, e.g., Chopra and Roberts (2001) Microbiol. & Mol. Biol. Rev. 65:232-260).

In some embodiments, the antimicrobial agent for use with the presently disclosed methods and compositions is a penicillin antibiotic including, but not limited to, Amoxicillin/clarithromycin (available from Advancis Pharmaceutical Corp), Alexid (pivmecillinam) (available from Aristopharma Ltd.), Selexid (pivmecillinam) (available from Dong Wha Pharm. Ind. Co., Ltd.), and the like.

In other embodiments, the antimicrobial agent for use with the presently disclosed methods and compositions is a broad-spectrum bactericidal antibiotic, such as fosfomycin, and pharmaceutically acceptable salts thereof, including, Urizone Granules (fosfomycin trometamol) (available from Adcock Ingram Limited), Fosfomycin calcium (available from AXM Pharma Inc), Fosfomycin sodium (available from Dragon Pharmaceutical Inc), Monuril (fosfomycin tromethamol)/Monurol (available from Forest Laboratories Inc), Fosfomycin calcium (available from North China Pharmaceutical Group Corp), Monurol (fosfomycin tromethamol) (available from Pierre Fabre Medicament), Uridoz (fosfomycin trometamol) (available from THERABEL Group), Monuril/Monurol (fosfomycin tromethamol) (available from Zambon Group), Monuril/Gantrisin (sulfisoxazole) (available from Roche Holdings Ltd) and the like. Fosfomycin tromethamol is a synthetic, broad-spectrum, bactericidal antibiotic for oral administration. The bactericidal action of fosfomycin is due to its inactivation of the enzyme enolpyruvyl transferase, thereby irreversibly blocking the condensation of uridine diphosphate-N-acetylglucosamine with p-enolpyruvate, one of the first steps in bacterial cell wall synthesis. It also reduces adherence of bacteria to uroepithelial cells.

In some embodiments, the antimicrobial agent for use with the presently disclosed methods and compositions includes other antibiotics such as nitrofurantoin, including, Piyeloseptyl (nitrofurantoin) (available from Biofarma Pharmaceutical Industry Co. Inc.), Furadantine delay (nitrofurantoin) (available from Galenica Ltd.), Macrodantin (nitrofurantoin) (available from Geymonat S.p.A.), Furadantin Retard (nitrofurantoin) (available from Goldshield Group plc), Macrobid (nitrofurantoin) (available from Goldshield Group plc), Macrodantin (nitrofurantoin) (available from Goldshield Group plc), Nitrofurantoin Oral Suspension (available from Goldshield Group plc), Furantoina (nitrofurantoin) (available from Grupo Uriach), Nitrofurantoin (available from Laboratorio Teuto), Nitrofurantoin (available from Macleods Pharmaceuticals Limited), Nitrofurantoin monohydrate/macrocrystals (available from Mylan Laboratories Inc), Macrobid (nitrofurantoin monohydrate/macrocrystals) (available from Procter & Gamble Company), Macrodantin (nitrofurantoin macrocrystals) (available from Procter & Gamble Company), Niftran (nitrofurantoin modified release) (available from Ranbaxy Laboratories Ltd.), Nitrofurantoin monohydrate (available from Ranbaxy Laboratories Ltd.), Furadantin Oral Suspension (nitrofurantoin) (available from Sciele Pharma, Inc.), Furantine (nitrofurantoin) (available from Shalina Laboratories Pvt Ltd.), and Nitrofurantoin monohydrate/macrocrystals (available from Watson Pharmaceuticals Inc).

In other embodiments, the antimicrobial agent for use with the presently disclosed methods and compositions includes other antibiotics such as methenamine, including Methenamine mandelate (available from Actavis), Methenamine mandelate (available from United Research Laboratories (Mutual Pharmaceutical Company), Mandelamine (methenamine mandelate) (available from Warner Chilcott Limited), and the like, or combination therapies comprising hexamine (also referred to herein as methenamine), including Utira (hyoscyamine sulfate, methenamine, phenyl salicylate, sodium biphosphate, methylene blue) (available from Hawthorn Pharmaceuticals, Inc.), Hexamol (hexamine, theobromine, piperazine tartarate, quinic acid, sodabicarb, tartaric acid) (available from Efroze Chemical Industries), Urelle (phenyl salicylate, hyoscyamine sulfate, methenamine, sodium biphosphate) (available from Azur Pharma Limited), wherein phenyl salicylate acts as an analgesic, hyoscyamine sulfate is an antispasmodic which relieves spasm, methenamine and sodium biphosphate acts as antibacterial, and methylene blue has antiseptic properties; Urelle Plus (available from Azur Pharma Limited) is a combination therapy for urinary antiseptic and pain relief; Prosed DS (methenamine, phenyl salicylate, atropine sulfate and hyoscyamine sulfate) (available from Esprit Pharma, Inc.), Prosed EC (methenamine, phenyl salicylate, atropine sulfate and hyoscyamine sulfate) (available from Esprit Pharma, Inc.), Uricol (hexamine, piperazine citrate and khellin) (available from Pharco Pharmaceuticals Inc.).

In some embodiments, the antimicrobial agent for use with the presently disclosed methods and compositions includes dihydrofolate reductase inhibitors, including bacteriostatic antibiotics, such as trimethoprim, including Idotrim (trimethoprim) (available from Abigo Medical), Trimethoprim (available from BELCO Pharma), Proloprim (trimethoprim) (available from King Pharmaceuticals Inc), Trimethoprim (available from Sandoz International GmbH), Triprim (trimethoprim) (available from Sigma Pharmaceuticals Limited), Trimethoprim Tablets (available from Watson Pharmaceuticals Inc), and the like.

In further embodiments, the antimicrobial agent for use with the presently disclosed methods and compositions includes sulfa drugs. The term “sulfa drug” refers to a class of synthetic chemical substances derived from sulfanilamide called sulfonamides. This class includes several antibiotics, including sulfamethoxazole, sulfasalazine, sulfacetamide, and the like.

In some embodiments, the antimicrobial agent for use with the presently disclosed methods and compositions includes combination therapies, such as trimethoprim in combination with a sulfa drug, including, but not limited to Coptin (sulfadiazine and trimethoprim) (available from Axcan Pharma Inc), Cotrimoxazole (trimethoprim, sulphamethoxazole) (available from Jagsonpal Pharmaceuticals Ltd.), Septra (trimethoprim and sulfamethoxazole) (available from King Pharmaceuticals Inc), Chemix (sulphamethoxazole and trimethoprim) (available from Yung shin Pharmaceutical), Bactrim (sulfamethoxazole and trimethoprim)(available from Roche Holdings Ltd.), and the like.

In other embodiments, the therapeutic agent useful for treating a UTI that may be used as an additional active compound in the methods and compositions of the invention is Usept (available from Breckenridge Pharmaceutical, Inc.), which contains anti-infective, pain reliever, and antispasmodic ingredients. More particularly, Usept contains methenamine, phenyl salicylate, methylene blue, benzoic acid, atropine sulfate and hyoscyamine sulfate as active ingredients. Methenamine degrades in an acidic urine environment releasing formaldehyde which provides bactericidal or bacteriostatic action. Phenyl salicylate releases salicylate, a mild analgesic for pain. Methylene blue possesses weak antiseptic properties. Benzoic acid has mild antibacterial and antifungal action. It also helps maintain an acid ph in the urine necessary for the degradation of methenamine. Atropine sulfate and hyoscyamine sulfate are parasympatholytic drugs which relax smooth muscles.

In further embodiments, the additional active compound useful for treating a UTI that may used in the methods and compositions of the invention is an analgesic, such as phenazopyridine, including phenazopyridine hydrochloride (available from Actavis), Pyridium (phenazopyridine hydrochloride) (available from Pfizer Limited), Sedural (phenazopyridine hydrochloride) (available from Rekah Pharmaceutical Industry Ltd.), Phenazopyridine (available from Sandoz International GmbH), Phenazopyridine hydrochloride (available from Sunrise Pharmaceutical, Inc.), Azo-gesic (phenazopyridine) (available from United Research Laboratories (Mutual Pharmaceutical Company), Phenazopyridine (available from United Research Laboratories (Mutual Pharmaceutical Company), Pyridium (phenazopyridine hydrochloride) (available from Warner Chilcott Limited), and the like.

In another embodiment, the additional active compound useful for treating a UTI that may used in the methods and compositions of the invention is Uroprin (phenylazodiaminopyridine hydrochloride) (available from Yung shin Pharmaceutical), which contains phenylazodiaminopyridine hydrochloride (an azo dye). When excreted in the urine it exerts a topical analgesic or local anesthetic effect on the mucosa of the urinary tract. Uroprin is used as a urinary tract analgesic for the symptomatic relief of pain, burning, urgency, frequency, and other discomforts resulting from irritation of the lower urinary tract mucosa.

In some embodiments, the additional active compound useful for treating a UTI that may used in the methods and compositions of the invention is a Urinary Tract Infection Vaccine (available from Medlmmune Inc), or female estrogen hormones, such as Auroclim (estradiol valerate) (available from Juste S.A.Q.F.), which contain the principal intracellular human estrogen. Such hormones enter target cells freely and interacts with a target cell receptor. When the estrogen receptor has bound its ligand it can enter the nucleus of the target cell, and regulate gene transcription which leads to formation of messenger RNA. The mRNA interacts with ribosomes to produce specific proteins that express the effect of estradiol upon the target cell.

Other agents are currently under development for treating urinary tract infections and may also be used an additional active compounds within the methods and compositions of the invention. Such agents include:

a) Pyrrhocoricin analogs, such as CHP-105 (available from Chaperone Technologies, Inc.), which is being developed for the treatment of complicated urinary tract infections. It exhibits high potency versus multi-drug resistant variants of major uropathogens in vitro under conditions where standard antibiotics remain inactive, and significantly reduces bacterial count.

b) B-060703 (available from ConjuGon Inc.) is a genetically-modified bacterium that, through the process of conjugation delivers engineered genes from harmless donor bacteria to targeted, unwanted bacteria. The genes then are expressed in the target and form antibacterial gene products, which kill the unwanted bacteria through multiple and redundant mechanisms.

c) NXB-4221 (available from Nymox Pharmaceutical Corporation) is an antibacterial agent being developed for the treatment of difficult chronic and persistent urinary tract infection.

d) UK-369003 (available from Pfizer Inc) is a phosphodiesterase V inhibitor being developed for the treatment of Lower Urinary Tract Symptoms.

e) TG-873870 Oral (nemonoxacin) (available from Procter & Gamble Company), and TG-873870 (Oral) (available from TaiGen Biotechnology) is a novel non-fluorinated quinolone antibiotic and a bacterial topoisomerase inhibitor. It is being developed as an oral formulation for the treatment of urinary tract infections.

f) XP19J/rUTI (available from Xanodyne Pharmaceuticals, Inc. (formerly Xanodyne Pharmacal)) is being developed for the treatment of urinary tract infections.

g) Lactin-V (available from Osel Inc.) is a vaginal capsule containing a natural human bacterium Lactobacillus crispatus being developed for the treatment of recurrent urinary tract infection.

h) Furaginum (furagin) (available from Adamed Sp. z o.o.), is an antibacterial agent used for the treatment of acute and chronic urinary tract infections.

i) Macmiror (nifuratel) (available from CSC Pharmaceuticals Handels GmbH) contains nifuratel as the active ingredient which is a furane-derivative with a strong trichomonicidal activity and is used for the treatment of urinary tract infections, mixed vulvovaginal infections, intestinal amebiasis and lambliasis.

j) K-CIT (available from Dr Reddys Laboratories Ltd) is a combination of potassium citrate monohydrate and citric acid, which is a urinary anti-infective drug. Potassium citrate is metabolised to potassium bicarbonate and acts as a systemic alkaliser. Citrate forms ionic complexes of calcium and reduces ionic calcium concentration. It prevents the formation of urinary stones composed of uric acid and cystine and is used for the prevention of recurrence of urinary stones, relief from pain and burning micturition and renal tubular acidosis.

k) Urolene Blue (available from Esprit Pharma, Inc.), contains methylene blue, which is a mild urinary antiseptic and stimulant to mucous surfaces. It is used as a genitourinary antiseptic in cystitis and urethritis both by internal administration and by irrigation.

l) Acimethin (L-methionine) (available from Galenica Ltd.), contains L-methionine, a natural amino acid, which is involved in the acidification of alkaline urine.

m) Flavoxate Hydrochloride Tablets (available from Impax Laboratories Inc), is a non-specific, direct-acting, smooth muscle relaxant and a muscarinic receptor antagonist and is indicated for the symptomatic relief of dysuria, urgency, nocturia, vesical supra-pubic pain, frequency and incontinence as may occur in cystitis, prostatitis, urethritis, urethro-cystitis and urethrotrigonitis.

n) Lithostat (acetohydroxamic Acid) (available from Mission Pharmacal Company), contains acetohydroxamic acid as an active ingredient and reversibly inhibits the bacterial enzyme ureage, thereby inhibiting hydrolysis of urea and production of ammonia in urine infected with urea-splitting organisms. Further, acetohydroxamic acid is an ammonia detoxicant that is used primarily to treat chronic urinary tract infections.

o) Uro-Vaxom (available from OM PHARMA), is an immunomodulator containing lyophilized bacterial lysates of Escherichia coli. Uro-Vaxom stimulates T-lymphocytes, induces production of endogenous interferon and increases sIgA level in urine.

p) Geocillin (carbenicillin indanyl sodium) (available from Pfizer Inc), contains carbenicillin indanyl sodium as an active ingredient which is a semisynthetic penicillin that exerts its antibacterial activity by interference with final cell wall synthesis of susceptible bacteria.

q) Urobiotic-250 (available from Pfizer Inc), contains oxytetracycline hydrochloride, phenazopyridine hydrochloride, and sulfamethizole as active ingredients. Oxytetracycline belongs to a group of antibiotics called tetracyclines which inhibit the growth of a wide variety of bacteria by interfering with the production of proteins that the bacteria need to multiply and divide. Phenazopyridine hydrochloride has a specific local analgesic effect in the urinary tract and relieves burning and pain. Sulfamethizole, a sulfonamide antibiotic is a competitive inhibitor of bacterial para-aminobenzoic acid (PABA), a substrate of the enzyme dihydropteroate synthetase.

r) Sunrise Urinary Antiseptic (available from Sunrise Pharmaceutical, Inc.) is a film coated tablet, which is a urinary antiseptic.

s) Spasmo-Euvernil (sulfacarbamide, phenazopyridine) (available from TTY BioPharm) contains sulfacarbamide and phenazopyridine. Phenazopyridine is an azo dye that exerts a topical analgesic effect on the mucosa of the urinary tract. Sulfacarbamide is an antibacterial agent.

t) Pyridium plus (phenazopyridine hydrochloride, hyoscyamine hydrobromide, butabarbital) (available from Warner Chilcott Limited), contains phenazopyridine hydrochloride, hyoscyamine hydrobromide and butabarbital. Phenazopyridine is an azo dye that exerts a topical analgesic effect on the mucosa of the urinary tract. Hyoscyamine hydrobromide is an antispasmodic which relieves spasm. Butabarbital has sedative and calming effects.

u) Nice (nitroxoline) (available from Yung shin Pharmaceutical) contains nitroxoline and is a urinary antibiotic agent active against susceptible gram-positive and gram-negative organisms commonly found in urinary tract infections.

v) Clarithromycin XL (available from Advancis Pharmaceutical Corp), is an extended release formulation of a semi-synthetic macrolide antibiotic chemically related to erythromycin. It interferes with the protein synthesis of bacteria by binding to the 505 subunit of the bacterial ribosome, inhibiting the translocation of peptides.

w) Cubicin/Cidecin (daptomycin) (available from Cubist Pharmaceuticals Inc and Medison Pharma Ltd.) contains daptomyin, a novel cyclic lipopeptide antibiotic derived from a fermentation product of Streptomyces roseosporus. It is being developed for the treatment of serious and life-threatening bacterial infections and Complicated Urinary Tract Infections.

x) Gantanol (sulfamethoxazole) (available from Roche Holdings Ltd), is an intermediate-dosage antibacterial sulfonamide available in tablets. Each tablet contains 0.5-g sulfamethoxazole used in the treatment of urinary tract infections.

y) Kukje ribostamycin sulfate injection (available from KUKJE PHARMA IND CO LTD), which is an aminoglycoside antibiotic obtained from cultures of Streptomyces ribosidificus and inhibits bacterial protein synthesis. It is used in the treatment of gonorrheal urethritis, pyelonephritis, cystitis, cholecystitis, peritonitis, respiratory infections, furuncle and abscess.

In some embodiments, the additional active compounds within the methods and compositions of the invention include an herbal or natural product remedy. Such remedies include Herbion (available from Krka, d. d.), which is available as oral drops. Wantex (available from ASIA PHARMACEUTICAL INDUSTRIES) contains alpha pinine, beta pinine, camphene, boneol, anethol, fenchone and cineole. Cranberry Caplets (available from Perrigo Company), cranberry juice (see, e.g., Avorn et al. (1994) JAMA 271:751-754). Cranberries contain a type of flavonoid that is capable of defeating the bacteria that cause urinary tract infections. Cranberry caplets are made from concentrated cranberry juice, minus the fiber for preventing and treating urinary tract infections. Uricalm (available from Alva-Amco Pharmacal Cos., Inc.) contains cranberry and can provide relief of pain, burning and sensation of urgency caused by urinary tract infections. UriKhus (available from Lupin Ltd), is a systemic urinary alkalizer which contains kulattha (Dolichos biflorus), kankola (Piper cubeba), pashanabheda (Bergenia ligulata), varuna (Crataeva nurvala), sariva (Hemidesmus indicus), punarnava (Boerhaavia diffusa) and ushira (Vetivera zizanioides) and is used in the management of urinary tract infections as an adjuvant. Aqualibra (available from MEDICE) contains three vegetable active substances namely Ononis spinosa (spiny restharrow), Java Tea (Orthosiphon), and Solidago virgaurea (Goldenrod). Rowatinex (available from Amoun Pharmaceuticals Co. S.A.E), contains anethol, borneol, fenchone, alpha and beta pinene, D-camphene and cineol in olive oil. Ural capsule (available from VASU PHARMACEUTICALS PVT. LTD.), contains lithotryptic agents, such as pashanbhed, gokshurak, and hajrool yahood bhasma, and diuretics, such as kulthi and chandraprabha.

In further embodiments, the additional active compounds within the methods and compositions of the invention include drugs that block adherence of bacteria to the bladder wall. U.S. Pat. No. 5,180,715 to Parsons et al. discloses that pentosan polysulfate, a glycoaminoglycan, reduces adherence of bacteria to the bladder wall (See also Parsons et al. (1988) Infection and Immunity, 56: 1341-1343, and references cited therein). Further, pentosan polysulfate sodium (ELMIRON®, Ortho-McNeil Pharmaceutical, Inc., Raritan, N.J.) is currently FDA approved for relief of bladder pain or discomfort associated with interstitial cystitis. Other drugs that block adherence of bacteria to the bladder wall include mannose containing small molecules such as D-mannose, a methyl mannopyranoside, and aromatic alpha-glycosides of mannose such as 4-methylumbelliferyl alpha-mannoside and p-nitro-o-chlorophenyl alpha-mannoside (see Ruggieri et al. (1985) Urol. Res. 13:79-84; Firon et al. (1987) Infect. Immun. 55:472-476).

### Cholesterol Lowering Drugs

In some embodiments, the additional active compound is a cholesterol lowering drug. It has recently been shown that drugs that lower or disrupt cholesterol in membranes of mouse bladders reduce intracellular carriage of E. coli (Duncan et al. (2004) J. Biol. Chem. 279:18944-18951). Cholesterol lowering drugs include statins, bile resins, nicotinic acid (niacin), fibric acids (fibrates), and cholesterol absorption inhibitors.

Statins are HMG-CoA reductase inhibitors that lower cholesterol by inhibiting the enzyme HMG-CoA reductase, which is the rate-limiting enzyme of the mevalonate pathway of cholesterol synthesis. Representative statins include, but are not limited to, Atorvastatin (LIPITOR®, Pfizer Inc., New York, N.Y.), fluvastatin (LESCOL®, Novartis Pharmaceuticals Corporation, East Hanover, N.J.), lovastatin (MEVACOR®, Merck & Co., Inc. Whitehouse Station, N.J.), mevastatin, pitavastatin (Livalo (JP), Pitava (IN), pravastatin (PRAVACHOL®, Bristol-Myers Squibb Company, New York, N.Y.), rosuvastatin (CRESTOR®, AstraZeneca Pharmaceuticals, LP, Wilmington, Del.), simvastatin (ZOCOR®, Merck & Co., Inc.), and ezetimibe plus simvasatin combination therapy (VYTORINT™, Merck/Schering-Plough Pharmaceuticals, Whitehouse Station, N.J./Kenilworth, N.J.).

Bile resins, also referred to as bile acid-binding drugs, bind with bile acids in the intestines to form insoluble complexes, which are excreted with the stool. When bile acids are excreted, plasma cholesterol is converted to bile acid to normalize bile acid levels. This conversion of cholesterol lowers plasma cholesterol concentrations. Three types of therapeutic agents are in this class: cholestyramine (PREVALITE® (Upsher-Smith Laboratories, Minneapolis, Minn.); colestipol (COLESTID®, Pfizer); and Colesvelam (WELCHOL®, Daiichi Sankyo, Inc., Parsippany, N.J.).

Nicotinic acid (niacin, e.g., NIASPAN®, Kos Pharmaceuticals, Cranbury, N.J.) functions after conversion in the body to nicotinamide adenine dinucleotide (NAD) in the NAD coenzyme system. Niacin, e.g., in prescription slow release form, can be used to lower triglycerides and LDL cholesterol and raise HDL cholesterol.

Fibric acid derivatives, also referred to as “fibrates” reduce triglyceride production and remove triglycerides from circulation. More particularly, fibrates affect the actions of key enzymes in the liver, enabling the liver to absorb more fatty acids, thus reducing production of triglycerides. These triglyceride-lowering drugs also increase the levels of “good” high-density lipoprotein (HDL) cholesterol. Fibrates include gemfibrozil and fenofibrate.

Gemfibrozil (LOPID®, Pfizer, Inc., New York, N.Y.) is an antihyperlipidemic agent, which lowers the blood levels of triglycerides and low-density lipoprotein (LDL) (“bad”) cholesterol by reducing their production by the liver. It also increases blood levels of high-density lipoprotein (HDL) (“good”) cholesterol.

Fenofibrate (TRICOR®, Abbott Laboratories, Abbott Park, Ill.) activates peroxisome proliferator activated receptor α (PPAR α). Through this mechanism, fenofibrate increases lipolysis and elimination of triglyceride-rich particles from plasma by activating lipoprotein lipase and reducing production of apoprotein C-III, an inhibitor of lipoprotein lipase activity.

Cholesterol absorption inhibitors lower blood cholesterol by inhibiting its absorption in the small intestine. One such cholesterol absorption inhibitor is Ezetimibe (ZETIA®, Merck/Schering-Plough Pharmaceuticals, North Wales, Pa.). Ezetimibe's mechanism of action differs from other classes of cholesterol-reducing medications in that it localizes to the brush border of the small intestine where it inhibits absorption of cholesterol, decreasing the delivery of intestinal cholesterol to the liver. This decreases cholesterol stores within the liver and ultimately increases clearance of cholesterol from the blood. Ezetimibe can be administered alone or with a statin.

### Chemical Definitions

While the following terms are believed to be well understood by one of ordinary skill in the art, the following definitions are set forth to facilitate explanation of the presently disclosed subject matter. Unless otherwise defined, all technical and scientific terms used herein have the same meaning as commonly understood by one of ordinary skill in the art to which this presently described subject matter belongs.

Throughout the specification and claims, a given chemical formula or name shall encompass all optical and stereoisomers, as well as racemic mixtures where such isomers and mixtures exist.

When the term “independently selected” is used, the substituents being referred to (e.g., R groups, such as groups R₁, R₂, and the like, or groups X₁ and X₂), can be identical or different. For example, both R₁ and R₂ can be substituted alkyls, or Ri can be hydrogen and R₂ can be a substituted alkyl, and the like.

A named “R” or “X” group will generally have the structure that is recognized in the art as corresponding to a group having that name, unless specified otherwise herein. For the purposes of illustration, certain representative “R” and “X” groups as set forth above are defined below. These definitions are intended to supplement and illustrate, not preclude, the definitions that would be apparent to one of ordinary skill in the art upon review of the present disclosure.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl)hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

The term “cycloalkylalkyl,” as used herein, refers to a cycloalkyl group as defined hereinabove, which is attached to the parent molecular moiety through an alkyl group, also as defined above. Examples of cycloalkylalkyl groups include cyclopropylmethyl and cyclopentylethyl.

The terms “cycloheteroalkyl” or “heterocycloalkyl” refer to a non-aromatic ring system, such as a 3- to 7-member substituted or unsubstituted cycloalkyl ring system, including one or more heteroatoms, which can be the same or different, and are selected from the group consisting of N, O, and S, and optionally can include one or more double bonds. The cycloheteroalkyl ring can be optionally fused to or otherwise attached to other cycloheteroalkyl rings and/or non-aromatic hydrocarbon rings. Representative cycloheteroalkyl ring systems include, but are not limited to pyrrolidinyl, pyrrolinyl, imidazolidinyl, imidazolinyl, pyrazolidinyl, pyrazolinyl, piperidyl, piperazinyl, indolinyl, quinuclidinyl, morpholinyl, thiomorpholinyl, thiadiazinanyl, tetrahydrofuranyl, and the like.

The term “alkenyl” as used herein refers to a straight or branched hydrocarbon of a designed number of carbon atoms containing at least one carbon-carbon double bond. Examples of “alkenyl” include vinyl, allyl, 2-methyl-3-heptene, and the like.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a straight or branched hydrocarbon of a designed number of carbon atoms containing at least one carbon-carbon triple bond. Examples of “alkynyl” include propargyl, propyne, and 3-hexyne.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

The term “heteroaryl” refers to an aromatic ring system, such as, but not limited to a 5- or 6-member ring system, including one or more heteroatoms, which can be the same or different, and are selected from the group consisting of N, O, and S. The heteroaryl ring can be fused or otherwise attached to one or more heteroaryl rings, aromatic or non-aromatic hydrocarbon rings, or heterocycloalkyl rings. Representative heteroaryl ring systems include, but are not limited to, pyridyl, pyrimidyl, pyrrolyl, pyrazolyl, azolyl, oxazolyl, isoxazolyl, oxadiazolyl, thiazolyl, isothiazolyl, imidazolyl, furanyl, thienyl, quinolinyl, isoquinolinyl, indolinyl, indolyl, benzothienyl, benzothiazolyl, enzofuranyl, benzimidazolyl, benzisoxazolyl, benzopyrazolyl, triazolyl, tetrazolyl, and the like.

A structure represented generally by the formula, wherein the ring structure can be aromatic or non-aromatic:

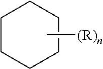

as used herein refers to a ring structure, for example, but not limited to a 3-carbon, a 4-carbon, a 5-carbon, a 6-carbon, and the like, aliphatic and/or aromatic cyclic compound, including a saturated ring structure, a partially saturated ring structure, and an unsaturated ring structure as defined herein, comprising a substituent R group, wherein the R group can be present or absent, and when present, one or more R groups can each be substituted on one or more available carbon atoms of the ring structure. The presence or absence of the R group and number of R groups is determined by the value of the integer n. Each R group, if more than one, is substituted on an available carbon of the ring structure rather than on another R group. For example, the structure above where n is 0 to 2 would comprise compound groups including, but not limited to:

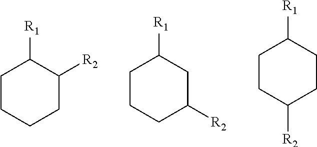

and the like.

A dashed line representing a bond in a cyclic ring structure indicates that the bond can be either present or absent in the ring. That is, a dashed line representing a bond in a cyclic ring structure indicates that the ring structure includes a saturated ring structure, a partially saturated ring structure, and an unsaturated ring structure.

When a named atom of an aromatic ring or a heterocyclic aromatic ring is defined as being “absent,” the named atom is replaced by a direct bond.

As used herein, the term “acyl” refers to an organic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl.

“Alkoxyl” refers to an alkyl-O— group wherein alkyl is as previously described. The term “alkoxyl” as used herein can refer to C₁₋₂₀ inclusive, linear, branched, or cyclic, saturated or unsaturated oxo-hydrocarbon chains, including, for example, methoxyl, ethoxyl, propoxyl, isopropoxyl, butoxyl, t-butoxyl, and pentoxyl.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

The term “alkyl-thio-alkyl” as used herein refers to an alkyl-5-alkyl thioether, for example, a methylthiomethyl or a methylthioethyl group.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl. “Aryloxycarbonyl” refers to an aryl-O—CO— group. Exemplary aryloxycarbonyl groups include phenoxy- and naphthoxy-carbonyl. “Aralkoxycarbonyl” refers to an aralkyl-O—CO— group. An exemplary aralkoxycarbonyl group is benzyloxycarbonyl.

“Carbamoyl” refers to an H₂N—CO— group. “Alkylcarbamoyl” refers to a R′RN—CO— group wherein one of R and R′ is hydrogen and the other of R and R′ is alkyl and/or substituted alkyl as previously described. “Dialkylcarbamoyl” refers to a R′RN—CO— group wherein each of R and R′ is independently alkyl and/or substituted alkyl as previously described.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

The term “alkylamino” refers to an —NHR group wherein R is an alkyl group and/or a substituted alkyl group as previously described. Exemplary alkylamino groups include methylamino, ethylamino, and the like.

“Dialkylamino” refers to an —NRR′ group wherein each of R and R′ is independently an alkyl group and/or a substituted alkyl group as previously described. Exemplary dialkylamino groups include ethylmethylamino, dimethylamino, and diethylamino.

“Acylamino” refers to an acyl-NH— group wherein acyl is as previously described. “Aroylamino” refers to an aroyl-NH— group wherein aroyl is as previously described.

The term “carbonyl” refers to the —(C═O)— group.

The term “carboxyl” refers to the —COOH group.

The terms “halo”, “halide”, or “halogen” as used herein refer to fluoro, chloro, bromo, and iodo groups.

The term “hydroxyl” refers to the —OH group.

The term “hydroxyalkyl” refers to an alkyl group substituted with an —OH group.

The term “mercapto” refers to the —SH group.

The term “oxo” refers to a compound described previously herein wherein a carbon atom is replaced by an oxygen atom.

The term “nitro” refers to the —NO₂ group.

The term “thio” refers to a compound described previously herein wherein a carbon or oxygen atom is replaced by a sulfur atom.

The term “sulfate” refers to the —SO₄ group.

As used herein, an “analog” refers to a chemical compound in which one or more individual atoms or functional groups of a parent compound have been replaced, either with a different atom or with a different functional group. For example, thiophene is an analog of furan, in which the oxygen atom of the five-membered ring is replaced by a sulfur atom.

As used herein, a “derivative” refers to a chemical compound which is derived from or obtained from a parent compound and contains essential elements of the parent compound but typically has one or more different functional groups. Such functional groups can be added to a parent compound, for example, to improve the molecule's solubility, absorption, biological half life, and the like, or to decrease the toxicity of the molecule, eliminate or attenuate any undesirable side effect of the molecule, and the like. An example of a derivative is an ester or amide of a parent compound having a carboxylic acid functional group.

The following examples are offered by way of illustration and not by way of limitation.

### EXPERIMENTAL

### Example 1

### Cyclic AMP-regulated Exocytosis of Escherichia coli from Infected Bladder Epithelial Cells

### Background

The superficial bladder epithelium is a powerful barrier to urine and also serves as a regulator of bladder volume, which is achieved by apical exocytosis of specialized fusiform vesicles during distension of the bladder. The present example shows that type 1 fimbriated uropathogenic Escherichia coli (UPEC) circumvents the bladder barrier by harboring in these Rab27b/CD63-positive and cAMP-regulatable fusiform vesicles within bladder epithelial cells (BECs). Incorporation of UPEC into BEC fusiform compartments enabled bacteria to escape elimination during voiding and to re-emerge in the urine as the bladder distended. Notably, treatment of UPEC-infected mice with a drug that increases intracellular cAMP and induces exocytosis of fusiform vesicles reduced the number of intracellular E. coli.

The urinary tract is one of the major mucosal sites for microbial colonization. The pathogen most effective at overcoming the mucosal barriers is uropathogenic E. coli (UPEC), the causative agent of 90% of UTIs (See Hooton & Stamm (1997) Infect. Dis. Clin. North Am. 11:551-581; Svanborg & Godaly (1997) Infect. Dis. Clin. North Am. 11:513-529). The success of E. coli as an uropathogen is linked to the expression of type 1 fimbriae. These filamentous appendages enable UPEC both to bind and to invade the superficial bladder epithelial cells (BECs) lining the bladder lumen (See Martinez et al. (2000) EMBO J. 19: 2803-2812; Duncan et al. (2004) J. Biol. Chem. 279:18944-18951). Bacterial invasion follows the binding of type 1 fimbriae to uroplakin 1a, a major component of the large scallop-shaped plaques found on the apical surface of superficial BECs (See Min et al. (2002) J. Mol. Biol. 317:697-706; Lewis (2000) Am. J. Physiol. Renal Physiol. 278:F867-F874; Hu et al. (2002) Am. J. Physiol. Renal Physiol. 283: F1200-F1207). These plaques arise from the fusion to the apical membrane of a dynamic pool of discoid-shaped vesicles, called fusiform vesicles (See Apodaca (2001) Urology 57:103-104; Apodaca (2004) Traffic 5:117-128). Exocytosis of fusiform vesicles helps regulate bladder surface area during the accumulation of urine. Urine accumulation triggers the initiation in BECs of fusiform vesicle exocytosis, by a cyclic AMP (cAMP)-dependent mechanism (See Apodaca (2001) Urology 57:103-104).

### Methods

Bacterial strains and cell lines. E. coli strain ORN103 with plasmid pSH2 (E.

coli type 1 fimbrial gene cluster, chloramphenicol resistance; see Orndorff and Falkow, (1984) J. Bacteriol. 159:736-744), and E. coli ORN103(pSH2) with plasmid pKEN-HcRed were used in the in vitro experimentation. The clinical UTI isolate E. coli CI5 was procured from a patient with an acute case of pyelonephritis. See Abraham et al. (1985) Infect. Immun. 48:625-628. ORN103(pSH2) (chloramphenicol; 100 μg/ml), HcRed ORN103(pSH2) (chloramphenicol, ampicillin; both 100 μg/ml), CI5 and Salmonella enterica serotype Typhimurium SL1344 were grown in Luria-Bertani broth with appropriate antibiotics. All colony counts were obtained by plating overnight at 37° C. on LB agar with the appropriate antibiotics.

The human bladder epithelial cell line 5637 (ATCC, HTB-9) was grown in RPMI 1640 (Invitrogen Corp., Carlsbad, Calif.) with 10% FBS (HyClone, Logan, Utah), 2 g/l sodium bicarbonate, 0.3 g/l L-glutamine, 2.5 g/l glucose, 10 mM HEPES, and 1 mM sodium pyruvate. All cells were cultured at 37° C. with 5% CO₂.

Construction of GFP-Rab27b—expressing BECs. The primers Hu-27bF 5′-GAT CTC GAG CTA TGA CCG ATG GAG ACT ATG AT-3′ and Hu-27bR 5′-GGT GGA TCC CTA GCA GAT ACA TTT CTT CTC TG-3′ (Integrated DNA Technologies, Coralville, Iowa) were used to amplify RAB27B from 5637 BECs by RT-PCR. The RT-PCR products were digested with XhoI and BamHI, and then ligated to XhoI/BamHI-digested pLEGFP-C1 (BD Biosciences, San Jose, Calif.). To generate BECs stably expressing GFP-RAB27B, the AmphoPack-293 cell line (BD Biosciences) was first used to produce viral particles and then these were used to infect 5637 BECs. Virus-infected cells were selected as recommended by the manufacturer.

Generation of Rab27b siRNA knockdown BECs. In this procedure, 2×10⁴ 5637 cells were transfected with 60 pmol of either Rab27b or randomly generated control siRNA duplexes (Ambion, Foster City, Calif.) for 24 h using lipofectamine 2000 (Invitrogen) and serum-free Opti-MEM medium (Invitrogen). After 40 h to 72 h, the transfected cells were used for invasion assays. Protein knockdown was confirmed by RT-PCR.

Bacterial internalization assay. In this assay, 5637 BECs were infected with ORN103(pSH2) E. coli diluted in RPMI 1640 medium at a multiplicity of infection (MOI) of 100:1. Cells were centrifuged for 5 min at 600 g, and incubated for 60 min at 37° C. Next, the cells were incubated with 100 μg/ml of gentamicin (Invitrogen) in RPMI medium for 30 min, and then with 10 μg/ml gentamicin in RPMI medium. At each time point, the cells were washed using PBS, solubilized with 0.1% Triton X-100 in PBS and plated for colony counts. All internalization assays had an n=12 wells of a 96-well plate.

Bacterial exocytosis assay. In this assay, 5637 BECs were infected with bacteria as above. Infection continued for 60 min, and then the cells were washed with culture medium (including 100 μg/ml gentamicin and 100 mM methyl α-D-mannopyranoside (Sigma, St. Louis, Mo.)) for an additional 30 min. The cells were then washed twice and then allowed to incubate for 1-4 h with fresh culture medium containing 100 mM methyl α-D-mannopyranoside. At each time point, the culture medium and one wash of 100 μl of culture medium with methyl α-D-mannopyranoside were collected and pooled. The inhibitors H89 (10 μM) and NiCl₂ (2 μM) were added during the gentamicin incubation and left in the culture medium. All homogenates and pooled culture medium were cultured for colony counts. All exocytosis assays had n=12 wells of a 96-well plate.

β-hexosaminidase Release Assay. In this assay, 5637 BECs were grown to confluence, washed with RPMI media, and incubated for 60 min with either 10 μM ionomycin (Sigma), 1 mM dibutyryl cAMP (Sigma), or 100 μM forskolin (Sigma). 5637 BECs also were infected with ORN103(pSH2) (100:1 MOI) in the presence or absence of 2 mM NiCl₂ (Sigma) or 10 μM H89 (Sigma). After a 60-min incubation, 30 μL of supernatant was removed, added to 10 μL of 4-nitrophenyl-N-acetyl-β-D-glucosaminide in citrate buffer (pH 4.5), and incubated for 1 hr at 37° C. The reaction was completed by the addition of 100 μL of 0.1M Na₂CO₃—NaHCO₃ buffer to the reaction mixture. Absorbance was read at 405 nm on a Tecan Sunrise microplate reader (Tecan Systems, Inc., San Jose, Calif.). All β-hexosaminidase release assays had an n=12 wells of a 96-well plate.

Fluorescent microscopy of cell lines. In this procedure, RAB27B-GFP 5637 BECs were incubated with either HcRed-ORN103(pSH2) or FITC-transferrin (Molecular Probes, Invitrogen, Carlsbad, Calif.) for 60 min at 37° C., and then fixed in 1% paraformaldehyde in PBS. The cells were then incubated with 50 mM NH₄Cl in PBS and blocked with 10% pig serum in PBS. Extracellular bacteria were labeled before cellular permeabilization with rabbit serum raised against E. coli CI5; for secondary labeling Alexa Fluor 350—conjugated goat antibody to rabbit IgG (Molecular Probes) was used. The cells were permeabilized and blocked with saponin buffer (0.05% saponin, 10 mM HEPES, 10 mM glycine, 10% pig serum). Coverslips were examined using a Nikon Eclipse TE200 microscope (Nikon Instruments, Melville, N.Y.) with appropriate filter sets. To determine intracellular bacterial colocalization with RAB27B-GFP, 50,000 BECs were scanned for intracellular bacteria. The present example identified 100 cells containing intracellular bacteria and then determined the percent of intracellular bacteria that colocalized with RAB27B-GFP within this subset.

In some embodiments, 5637 BECs were grown overnight on coverslips, incubated with either HcRed ORN103(pSH2) or FITC-transferrin (Molecular Probes) for between 5-60 min at 37° C., and fixed in 1% paraformaldehyde in PBS. The coverslips were incubated with 50 mM NH₄Cl in PBS and blocked with 10% pig serum in PBS. Extracellular bacteria were labeled prior to cellular permeabilization with rabbit serum raised against E. coli CI5 and secondary labeling with a goat anti-rabbit IgG conjugated with Alexa Fluor 350 (Molecular Probes). The cells were permeabilized and blocked with saponin buffer (0.05% saponin, 10 mM HEPES, 10 mM glycine, 10% pig serum). Primary antibodies in saponin buffer were incubated with the coverslips for 30 min at 4° C., washed three times with saponin buffer, and incubated with secondary antibodies in saponin buffer for 30 min at 4° C. Primary polyclonal antibodies to EEA1 (BD Biosciences) were revealed with goat anti-mouse IgG Alexa Fluor 488 F(ab′)2 (Molecular Probes) while primary mouse monoclonal antibodies to CD63 (BD Biosciences) were revealed with goat anti-mouse IgG Alexa Fluor 488 (Molecular Probes). Coverslips were mounted with Prolong Gold antifade reagent (Molecular Probes) and examined using a Nikon Eclipse TE200 microscope with a 4,6-diamidino-2-phenylindole filter set and a fluorescein filter set. Intracellular bacterial colocalization with cellular proteins was determined by scanning approximately 50,000 BECs for intracellular bacteria. After identification of 100 cells containing intracellular bacteria, the percent of intracellular bacteria that colocalized with CD63, EEA1 or transferrin was determined within this subset of cells.

In vivo bladder infection. In this procedure, 8-week-old female BALB/c mice were anesthetized with sodium pentobarbital and then inoculated transurethrally with 50 μl of either E. coli CI5 or a bacterial suspension (approximately 1.0×10⁸ CFU) suspended in PBS. After 2 h, the bladders were removed aseptically. Then the bladders were either bisected and fixed with 2% paraformaldehyde plus 2% gluteraldehyde in PBS (for transmission electron microscopy) or frozen for immunofluorescence microscopy. For the 2-h-long infections with E. coli and S. enterica SL1344, BALB/c mice were infected as above. After 2 h, the mice were intraperitoneally injected with 100 μl PBS (with or without 10 mg/kg forskolin) and intravesicularly instilled, for 1 h, with 100 μg/ml gentamicin in PBS (with or without 100 μM forskolin). For the 24-h-long infections, BALB/c mice were infected. After 24 h, the mice were intravesicularly instilled with 50 μl of PBS (with or without 100 μM forskolin) for 1 h. The 100 μM forskolin in PBS was replaced by 100 μg/ml gentamicin in PBS for 30 min. The bladders were then removed aseptically and homogenized them in 0.1% Triton X-100 in PBS. Homogenate dilutions were plated for colony counts. Two-hour E. coli CI5 infected mice had an n=6 and treated mice had an n=6. S. enterica SL1344 infected mice had an n=7 and treated mice had an n=7. Twenty-four-hour E. coli CI5 infected mice had an n=24 and treated mice had an n=25.

In vivo bladder infection with 3-d-long forskolin regimen. In this procedure, 8-week-old female C3H/HeJ mice were infected with E. coli CI5 as above. At 6, 24 and 48 h after infection, the mice were intraperitoneally injected with 100 μl PBS, with or without 10 mg/kg forskolin. At 72 h after infection, bladders were aseptically removed and plated for colony counts. E. coli CI5 infected mice had an n=13 and forskolin treated mice had an n=14.

IL-6 ELISA. In this assay, 8-week-old female C3H/HeJ mice were infected with E. coli CI5 as above. After 6 h, the mice were intraperitoneally injected with 100 μl of PBS, with or without 10 mg/kg forskolin. Urine was collected at 6 and 24 h after infection, using clean catch methods, and the samples were stored at −80° C. The concentration of IL-6 in the urine was determined using the mouse IL-6 ELISA kit (eBioscience, San Diego, Calif.) according to the manufacturer's protocol. The forskolin treated group had an n=10 and the saline treated group had an n=10.

Immunofluorescence of bladder sections. Balb/c mice were either infected with E. coli CI5 as above, or they were treated with either 100 μM forskolin or PBS for 1 h through a catheter. Frozen sections of these bladders were cut using standard methods. Each section was fixed in 100% ethanol for 20 min at −20° C., and blocked in PBS with 1% BSA for 1 h at 4° C. Sections were incubated overnight at 25° C. with primary antibodies to either Rab27b (Santa Cruz Biotechnology, Inc., Santa Cruz, Calif.) or UPIII (Santa Cruz Biotechnology), and washed. Sections were incubated for 1 h at room temperature with secondary antibodies, and washed. To visualize the nuclei, the sections were incubated with Hoechst-33258 for 5 min at room temperature and then washed. All dilutions and washes were with PBS and 1% BSA. Coverslips were viewed as described previously.

Transmission electron microscopy. Infected mouse bladders were stained in 1% OSO₄ and 2% uranyl acetate for 1 h and washed in between with ddH₂O. The bladders were dehydrated in increasing concentrations of acetone and finally embedded using a Poly/Bed 812 Embedding Media/DMP-30 Kit (Polysciences, Inc., Warrington, Pa.). The embedded bladder blocks were sectioned and examined using standard procedures.

Statistical analysis. Unpaired Student's t-tests were used to determine statistical significance. A Fisher test was used to determine the statistical significance of the drop in the number of CI5 infected Balb/c mice. The α-level for significance was 0.05.

### Results and Discussion

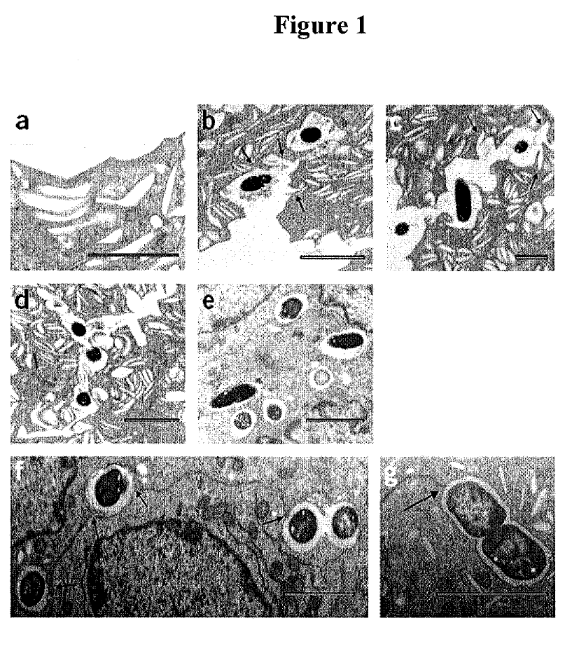

To elucidate the mechanism underlying UPEC invasion, the present example investigated the interactions between UPEC and the superficial epithelium in a mouse model. Mice were catheterized and intravesicularly instilled with the type 1 fimbriated UPEC strain CI5 for 2 h. Transmission electron microscopy showed fusiform vesicles in uninfected BECs (FIG. 1A) and bacterial invasion involving the participation of fusiform vesicles (FIG. 1B-1G). The attachment of E. coli CI5 to scalloped plaques on the BEC luminal surface coincided with the fusion of multiple fusiform vesicles proximal to the site of bacterial attachment (FIG. 1B). The coalescence of the fusiform vesicles appeared to produce a tubular invagination containing bacteria (FIG. 1C) and sequestered bacteria away from the lumen (FIG. 1D). A separate population of intracellular bacteria was observed within discrete compartments connected by tethers of membrane, possibly resulting from the collapse of the tubular invaginations around intracellular bacteria (FIG. 1E-G). This series of images suggests that E. coli invade superficial BECs through fusiform vesicles.

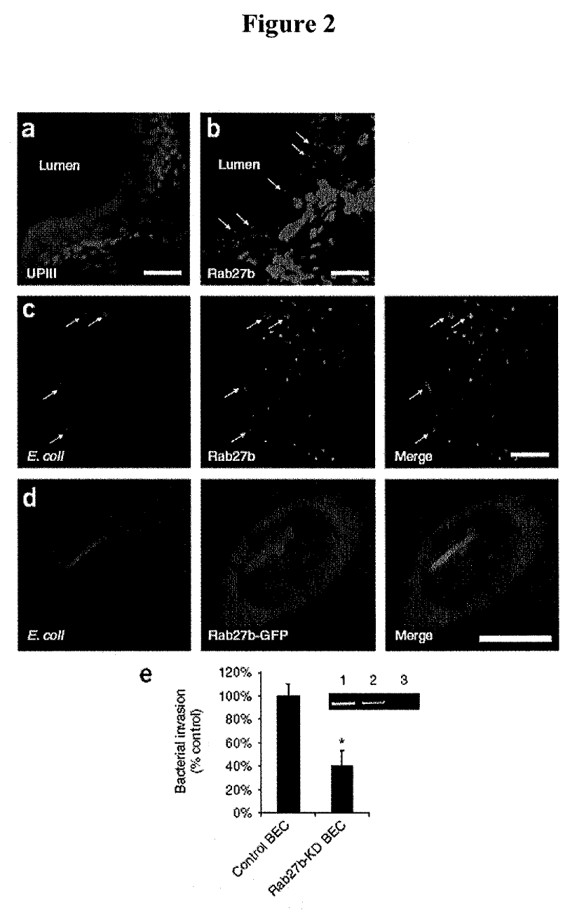

Frozen sections of infected bladder tissue were examined to determine whether intracellular vesicles harboring E. coli contained fusiform vesicle markers. To do this, antibodies to uroplakin III, a marker of superficial BECs (see Lewis (2000) Am. J. Physiol. Renal Physiol. 278:F867-F874), and Rab27b, a specific marker of fusiform vesicles were used (see Chen et al. (2003) Proc. Natl. Acad. Sci. USA 100:14012-14017). Uroplakin III-positive superficial BECs were distinguishable from other cells in the bladder (FIG. 2A), and the fusiform vesicles in these BECs expressed Rab27b (FIG. 2B). When the E. coli-infected tissue was costained with antibodies to E. coli and Rab27b, a strong association between intracellular bacteria and Rab27b was observed (FIG. 2C). These results suggest that UPEC invade superficial BECs by a mechanism involving fusiform vesicles, and, therefore, invasion may be regulated by signaling pathways in the host cell that control vesicular trafficking

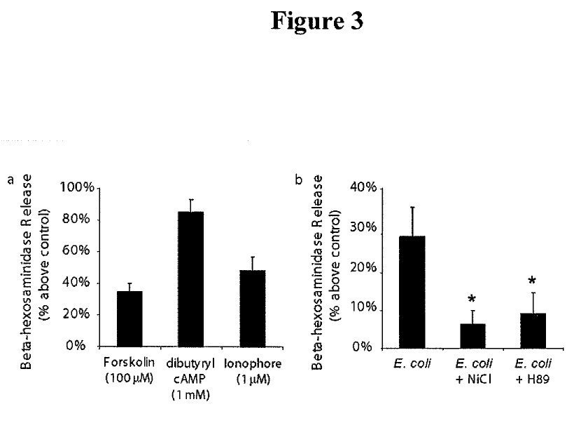

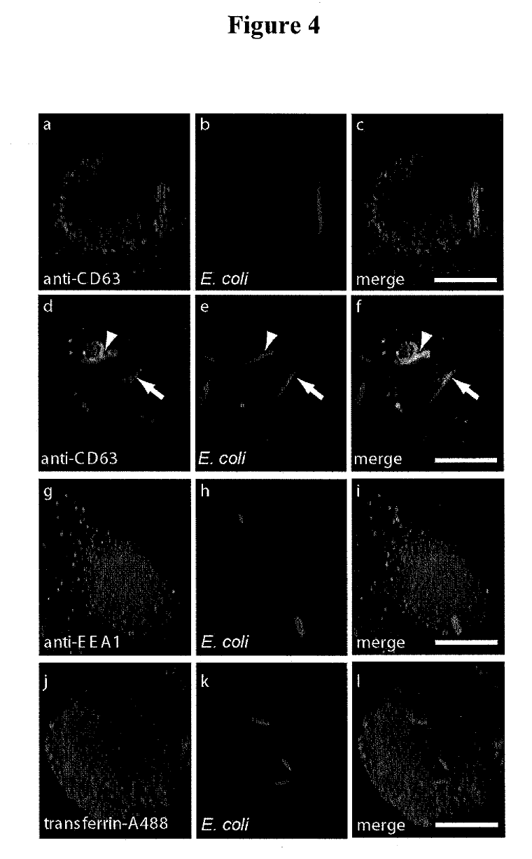

To further characterize the mechanism of bacterial invasion through fusiform vesicles, a type 1 fimbriated E. coli strain, ORN103(pSH2) (see Orndorff & Falkow (1984) J. Bacteriol. 159:736-744), and the human 5637 BEC line (HTB-9, ATCC) were investigated. Fusiform vesicles belong to a class of exocytic vesicles, known as secretory lysosomes, that are associated with Rab27b and are stimulated through both intracellular Ca²⁺ and cAMP flux (See Apodaca (2001) Urology 57:103-104; Chen et al. (2003) Proc. Natl. Acad. Sci. USA 100:14012-14017; Wang et al. (2003) Methods 30:207-217; Burgoyne & Morgan (2003) Physiol. Rev. 83:581-632). Distinct from classical lysosomes, secretory lysosomes lack degradative capacity; instead, they function as storage and secretory organelles (See Burgoyne & Morgan (2003) Physiol. Rev. 83:581-632). Fluctuations in intracellular Ca²⁺ or cAMP cause these vesicles to discharge their contents (Burgoyne & Morgan (2003) Physiol. Rev. 83:581-632). Using a β-hexosaminidase release assay, the present example found that 5637 BECs maintained a functional population of secretory lysosomes (FIG. 3). Additionally, 1 h of E. coli infection induced a Ca²⁺- and cAMP-sensitive β-hexosaminidase release from 5637 cells, suggesting that E. coli infection triggers the exocytosis of secretory lysosomes (FIG. 3). Furthermore, the present example shows that intracellular E. coli were contained within secretory lysosomes of 5637 BECs. Fluorescence microscopy revealed that 85% of internalized E. coli were housed in vesicles enriched in Rab27b (FIG. 2D). Intracellular E. coli-containing compartments also expressed an additional marker of secretory lysosomes (CD63), id., but lacked markers for early and recycling endosomes (early endosome antigen-1 and transferrin, respectively) (FIG. 4). To confirm the functional significance of Rab27b, siRNA was used to knockdown expression in 5637 cells. E. coli invasion was markedly inhibited in Rab27b-knockdown BECs (FIG. 2E). Therefore, E. coli infection of 5637 BECs initiated both the release of secretory lysosomes as well as the incorporation of bacteria into secretory lysosomes.

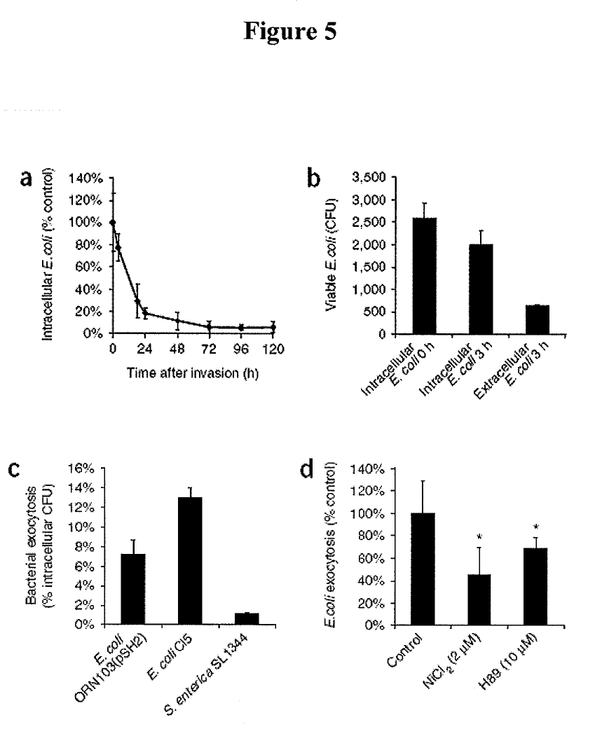

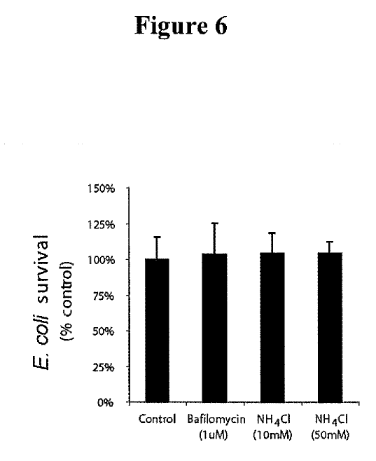

To investigate the fate of the internalized E. coli, 5637 BECs were infected for 1 h after which an antibiotic protection assay was used to quantify intracellular bacteria. Four hours after infection, a substantial decrease in the number of intracellular E. coli was observed and by 24 h, 80% of the intracellular bacteria were no longer present (FIG. 5A). Bacterial lysis of BECs and BEC degradation of invading bacteria were ruled out through a lactose dehydrogenase release assay and through an intracellular bacteria survival assay, respectively (FIG. 6). Without wishing to be bound to any one particular theory, these results suggest that the loss of intracellular E. coli resulted from the regulated exocytosis of bacteria-containing secretory lysosomes. In a modified antibiotic protection assay, the gentamicin-containing medium was replaced with fresh antibiotic-free medium after 1 h. Three hours after medium exchange, the number of E. coli found within the extracellular medium had increased to approximately 24% of the initial intracellular pool (FIG. 5B). This number correlated with a decrease in intracellular E. coli during the same time period. To determine if regulated exocytosis of bacteria within BECs was unique to E. coli, 5637 cells were infected with one of two E. coli strains (ORN103(pSH2) or CI5) and with Salmonella enterica serotype Typhimurium strain SL1344. E. coli CI5 exhibited a slightly faster escape than E. coli ORN103(pSH2) (13% versus 7%) (FIG. 5C). There was limited replication of E. coli ORN103(pSH2) or CI5 within BECs. Although invasion of S. enterica SL1344 was much greater than invasion of either of the E. coli strains, only a limited amount of Salmonella was exocytosed from 5637 BECs (≦2%) (FIG. 5C). As expected, inhibitors of Ca²⁺ flux and cAMP activity inhibited the escape of intracellular E. coli ORN103(pSH2) from 5637 BECs (FIG. 5D). After invasion of BECs, type 1 fimbriated E. coli were harbored within secretory lysosomes and this unique mechanism of invasion led to regulated bacterial exocytosis.

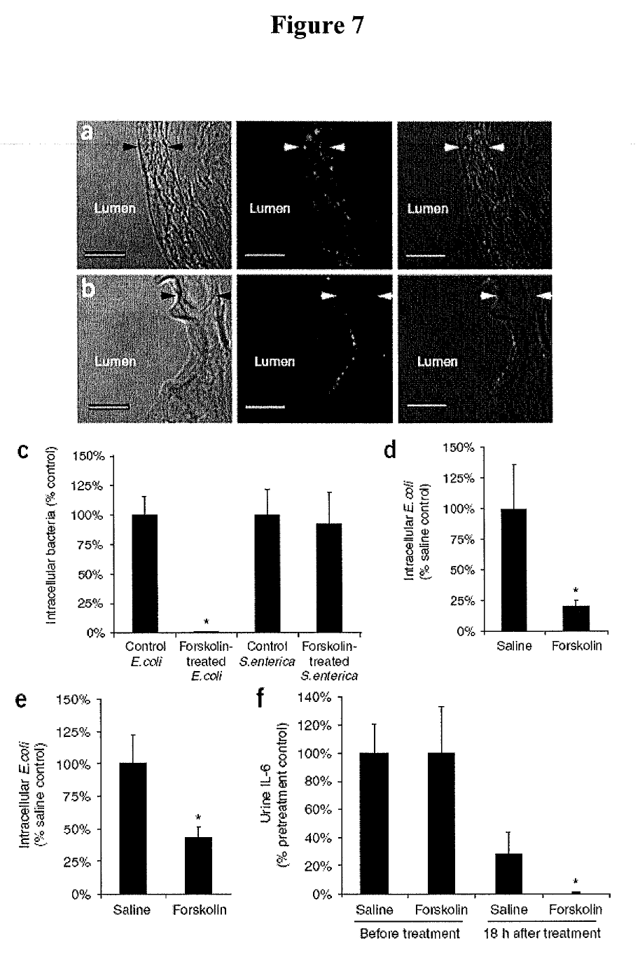

Whether modulators of cellular exocytosis can reduce the intracellular bacterial burden in an in vivo bladder infection also was examined. Forskolin, a powerful elevator of intracellular cAMP, triggers exocytosis of fusiform vesicles into the apical plasma membrane of BECs (See Wang et al. (2003) Methods 30:207-217; Truschel et al. (2002) Mol. Biol. Cell 13:830-846). Using antibodies to Rab27b, the translocation of fusiform vesicles to the apical plasma membrane of mouse bladders was detected following forskolin treatment (FIGS. 7A, 7B). Whether forskolin alters the bacterial invasion of mouse bladders also was investigated. Forskolin treatment eliminated over 99% of the intracellular E. coli CIS, whereas it had no effect on intracellular S. enterica SL1344 (FIG. 7C). Forskolin had no effect on bacterial viability (data not shown). These results suggest that UPEC are harbored in fusiform vesicles that can be exocytosed by drugs that increase intracellular cAMP levels.

The present example then investigated whether forskolin may have therapeutic potential as a treatment for UTI. To test this, female Balb/c mice were intravesicularly challenged with E. coli CIS and treated with intravesicular forskolin 24 h later. The present example showed a significant (79.4%; P 0.033) reduction in the number of E. coli CIS within forskolin-treated bladder compared to that in saline-treated controls (FIG. 7D). As Balb/c mice are naturally resistant to a prolonged UTI, the present example showed that after 24 h several mice had reduced bacterial burdens even in the absence of treatment. Nevertheless, 33% of the control mice harbored >10⁵ bacteria in their bladders, versus only 4.1% of the forskolin-treated mice (P≦0.01).

Next, C3H/HeJ mice were studied as they are known to develop prolonged colonization and infection of the bladder (See Schilling et al. (2001) J. Immunol. 166:1148-1155). Mice were intravesicularly challenged with E. coli CI5 and then intraperitoneally injected with either forskolin or saline at 6, 24 and 48 h after infection. After 6 h of infection, bacteria levels were greater than 10⁶ colony-forming units (CFU)/ml in all groups, indicative of a robust infection. Compared to the control mice, the average bacterial load within the bladders of forskolin-treated mice had decreased by 56% at 72 h (P≦0.03) (FIG. 7E).

Levels of interleukin (IL)-6 in the urine are a predominant inflammatory marker of UTI (See Carbone et al. (2002) Ann. NY Acad. Sci. 963:332-335; Uehling et al. (1999) World J. Urol. 17:351-358). Urine was collected from the mice 6 h after infection, just before forskolin treatment, and then at 18 h after forskolin treatment. IL-6 production was comparable between groups prior to forskolin treatment, whereas there was a significant reduction in IL-6 in the forskolin-treated group by 18 h after treatment (99.6% decrease, P≦0.0003) (FIG. 7F). Additionally, forskolin treatment alone did not alter IL-6 levels in uninfected mice. Taken together, the data showed that forskolin treatment reduces bacterial burden as well as markers of inflammation in a mouse model of UTI. Therefore, the role of forskolin as a means to improve bacterial clearance in vivo suggests that cAMP regulation may be a new target for future therapies against UTI.

Although there is growing evidence that UPEC invade the bladder epithelium (see Martinez et al. (2000) EMBO J. 19: 2803-2812; Duncan et al. (2004) J. Biol. Chem. 279:18944-18951; Mulvey et al. (1998) Science 282:1494-1497), it is unclear how these relatively innocuous type 1 fimbriated bacteria penetrate the highly impermeable, plaque-lined apical surface of superficial BECs. The present example indicates that E. coli invasion of BECs involves the active participation of the subapical pool of fusiform vesicles and the occupation of these compartments by the bacteria. Again, without wishing to be bound to any one particular theory, it appears that the association of E. coli with apical plaques on superficial BECs initiates spontaneous exocytosis of fusiform vesicles. As both fusiform vesicles and apical plaques are highly enriched in lipid raft membranes (see Vergara et al. (1974) J. Cell Biol. 61:83-94), these findings are in agreement with a previous report detailing a lipid raft-dependent mechanism of UPEC invasion (Duncan et al. (2004) J. Biol. Chem. 279:18944-18951). Additionally, the E. coli-induced exocytosis of secretory lysosomes seems similar to trypanosome invasion of epithelial cells. Trypanosoma cruzi invasion requires Ca²⁺-dependent exocytosis of secretory lysosomes and these bacteria eventually enter the cytosol through phagosomal lysis (See Rodriguez et al. (1999) J. Biol. Chem. 274:16754-16759; Tardieux et al. (1992) Cell 71:1117-1130; Andrews (1993) Biol. Res. 26:65-67). In contrast to T. cruzi, UPEC do not necessarily lyse the secretory lysosomes they inhabit, but rather use their exocytic character to cycle into the extracellular environment.

Urine represents a double-edged sword to bacteria. It provides a nutrient-rich environment in which the bacteria proliferate (See Anderson et al. (1977) Antimicrob. Agents Chemother. 12:559-562; Anderson et al. (1979) J. Clin. Microbiol. 10:766-771). Free-floating and loosely attached bacteria, however, are routinely eliminated during voiding. Consequently, the capacity to transiently invade the superficial bladder epithelium provides bacteria a safe way to avoid elimination. The present example adds to growing evidence supporting the importance of the intracellular life cycle of E. coli in the pathogenesis of UTI. The present example shows that in addition to the recently described intracellular bacterial communities (IBC), in which E. coli proliferate within a cytosolic biofilm-like aggregate (see Anderson et al. (2003) Science 301:105-107), a large subset of UPEC undergo transient invasion and cAMP-regulated exocytosis from membrane-bound compartments. As the invasion by E. coli occurs through fusiform vesicles, it is possible that these compartments are the initial site of IBC formation. Although a large percentage of intracellular bacteria are exocytosed out of the BECs, a small percentage of E. coli remain persistently intracellular (FIG. 5A). Again, without wishing to be bound to any one particular theory, these intracellular bacteria appear to be the source of IBCs. A recent publication has suggested that IBC-like aggregates arise from bacteria within similar compartments (See Eto et al. (2006) Cell. Microbiol. 8:704-717). These bacterial aggregates escape into the cytoplasm, progress into full-fledged IBCs and eventually re-emerge by erupting out of the BECs (See Mulvey et al. (2001) Infect. Immun. 69:4572-4579). The present example describes a new mechanism, in addition to IBC-mediated epithelial cell lysis, of bacterial re-emergence into the bladder lumen through regulated exocytosis. This controlled release of bacteria without destruction of superficial BECs probably occurs when the bladder distends as urine collects. This cycle of endocytosis and exocytosis can serve to sustain the infection and allows bacterial dissemination.

On the basis of morphological, biochemical and functional properties, it is likely that the intracellular compartments in superficial BECs that harbor UPEC are fusiform vesicles. The exocytosis of fusiform vesicles can be induced by agents that increase cAMP (Apodaca (2001) Urology 57:103-104) and the present example shows that the treatment of UPEC-infected mice with forskolin was effective in the clearance of UTI. Forskolin originates from the Asiatic herb Coleus forskohlii (see Seamon et al. (1981) Proc. Natl. Acad. Sci. USA 78:3363-3367). Forskolin and its derivatives are currently being used for the treatment of several ailments, including glaucoma, asthma, high blood pressure and leukemia (See Drewes et al. (2003) Phytochemistry 62:1019-1025; Wajima et al. (2002) Crit. Care Med. 30:820-826; Meyer et al. (1987) S. Afr. Med. J. 71:570-571; Styczynski & Wysocki (2006) Br. J. Haematol. 133:397-399). The present discovery that cAMP regulates the intracellular niche of UPEC has revealed a new and potentially effective strategy for combating UTI. The use of traditional antibiotics is plagued by their inadequate penetration of bladder epithelial cells, thus allowing intracellular bacteria to persist. Eliminating the small but important intracellular pool of bacteria may have large clinical benefits. Drugs that increase cAMP within BECs may speed the time-to-resolution of UTI and decrease morbidity. Pharmacotherapies that increase intracellular cAMP, combined with traditional antibiotics, may lead to large clinical benefits in refractory and recurrent UTIs.

### Example 2

### Effect of Forskolin on UPEC Invasion

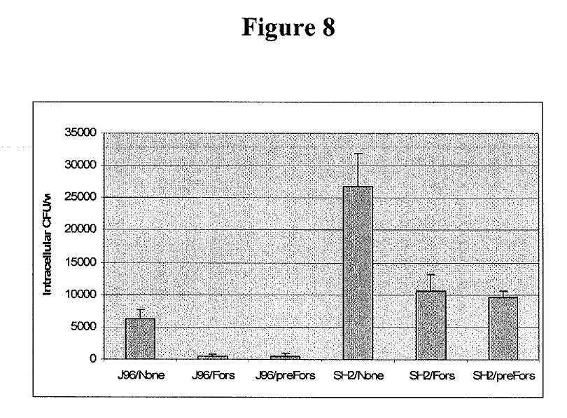

A gentamicin protection assay, including 5637 BECs on 96-well plates (40,000 cells/well), was used to determine the effect of forskolin on UPEC invasion. In this example, the UPEC was either J96 or E. coli ORN103(pSH2). 50 μM forskolin was administered as a pretreatment for 15 min before infection or administered at the same time as the bacteria. The results of this assay, in units of colony counts, are summarized in Table 2 and presented in FIG. 8, and demonstrate that forskolin treatment negatively affects UPEC invasion into BECs.

TABLE 2
Effect of Forskolin on UPEC Invasion

| | Dilution | 1 | 2 | 3 | 4 | Mean | SD |
|---|---|---|---|---|---|---|---|
| J96/None | 1:10 | 6140 | 4400 | 6540 | 8020 | 6275 | 1489 |
| J96/Forskolin | 1:10 | 800 | 640 | 460 | 280 | 545 | 225 |
| J96/Forskolin | 1:10 | 720 | 140 | 200 | 1080 | 535 | 447 |
| (pretreatment) | | | | | | | |
| SH2/None | 1:10 | 21660 | 27040 | 33840 | 24700 | 26810 | 5178 |
| SH2/Forskolin | 1:10 | 10980 | 8260 | 14140 | 8920 | 10575 | 2644 |
| SH2/Forskolin | 1:10 | 8860 | 9060 | 10740 | | 9553 | 1033 |
| (pretreatment) | | | | | | | |

### Example 3

### Efficacy of Forskolin in Reducing the CI5 E. coli Load within the Bladder

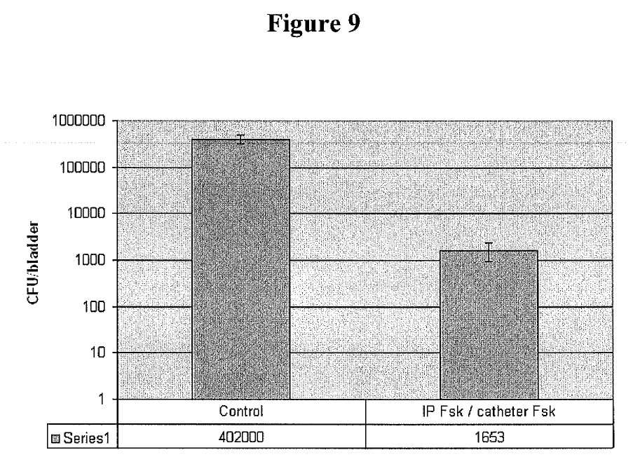

An in vivo gentamicin protection assay was used to determine the effect of forskolin on UPEC colonization of the bladder. In this example, UPEC strain CI5 was injected into the bladder via catheter to initiate a urinary tract infection. After 2 hours of infection, the mice were re-catheterized, the urine was removed, and the bladder was treated with 100 uM forskolin in PBS for 1 hour. After the forskolin treatment, 100 ug/ml gentamicin in PBS was injected into the bladder via catheter for 30 minutes. The bladders were then removed, washed in sterile PBS, and homogenized. The homogenates were plated on LB agar for overnight colony counts at various dilutions. Control mice were treated with PBS alone instead of forskolin plus PBS. The results of this assay, in units of colony counts, are summarized in Table 3 and averages are presented in FIG. 9. The top half of Table 3 represents the raw colony counts while the bottom half of Table 3 represents the calculated bladder load of UPEC strain CI5 based on the dilutions. These results demonstrate that forskolin treatment negatively affects UPEC colonization of the bladder, in vivo.

TABLE 3
Reduction of the CI5 E. coli Load within the
Bladder after 1 hr of Treatment with Forskolin.

| | | | IP | |
|---|---|---|---|---|
| | | | Fsk/Cather | |
| Mouse # | Control | Dilution | Fsk | |
| 1 | 153 | 1:100 | 91 | 1:1 |
| 2 | 21 | 1:1000 | 45 | 1:1 |
| 3 | 24 | 1:1000 | 112 | 1:1 |
| Average | 66 | | 83 | |

| | | | IP | | |
|---|---|---|---|---|---|
| | | | Fsk/Cather | | Percent |
| Mouse# | Control | Dilution | Fsk | | Control |
| 1 | 306000 | 1:100 | 1820 | 1:1 | 0.45% |
| 2 | 420000 | 1:1000 | 900 | 1:1 | 0.22% |
| 3 | 480000 | 1:1000 | 2240 | 1:1 | 0.56% |
| Average | 402000 | | 1653 | | 0.41% |
| Std. | 88386 | | 685 | | 0.17% |
| Dev. | | | | | |
| t-Test | | | 0.0014 | | |

### Example 4

### Effect of a Toll-Like Receptor Ligand on cAMP Levels in Bladder Cells

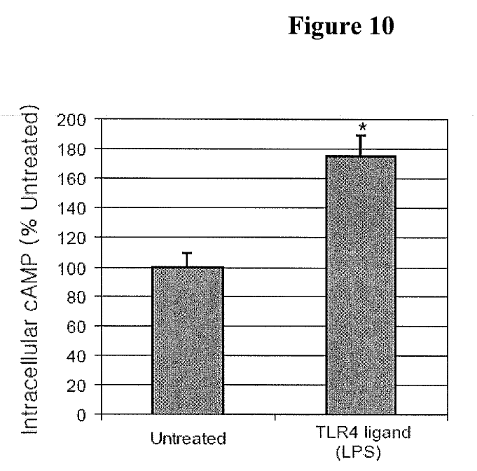

In this example, the effect of bacterial lipopolysccharide (LPS), a Toll-like Receptor 4 (TLR4) ligand, on intracellular levels of cAMP was assessed in human bladder epithelial cells (BECs). Methods are as described in Song et al. (2007) PLoS Pathog. 3(4):e60. BECs were seeded onto 6-well plates and grown overnight. The cells were treated with 100 mg/ml E. coli LPS for 6 h at 37° C., followed by washing four times with PBS to remove culture media, followed by an addition of 250 ml of 0.1 M HCl. After a 10 min incubation, the cell lysate was centrifuged and the supernatant was used directly in the cAMP assay. Intracellular concentrations of cAMP were determined using a cAMP enzyme immunoassay kit (Sigma) according to the manufacturer's instructions. As shown in FIG. 10, LPS elicited a clear and measurable increase in intracellular cAMP in human bladder epithelial cells.

### Example 5

### Effect of PDE Inhibitors, PKC Inducers, Toll-like Receptor Ligands, and Combination Therapies With Forskolin on cAMP Levels in Bladder Cells

As described above, treating mice with forskolin, an inducer of intracellular cAMP, triggered exocytosis of fusiform vesicles harboring bacteria and significantly reduced UTIs. Since cAMP is rapidly degraded in the cell by PDE inhibitors, it was hypothesized that PDE inhibitors could be used to mimic the same effects as forskolin in the urinary tract. Several PDE inhibitors are currently being used as treatment for other medical conditions, making them attractive candidates for the present study. Additionally, it was hypothesized that since the activity of PDE inhibitors were distinct from that of forskolin, a combination therapy employing forskolin along with PDE inhibitors could have an additive effect and significantly improve the therapeutic effects of either agent alone.

A number of in vitro studies were conducted employing human bladder epithelial cells (BECs) that had previously been validated to examine: 1) if PDE inhibitors reduce bacterial load in infected bladder cells; and 2) if combining a PDE inhibitor with forskolin would be more effective than either drug alone in the clearance of bladder cell infection in vitro.

Intracellular bacterial levels in BECs were measured in untreated BECs and in BECs treated with forskolin, various PDE inhibitors, and a combination of forskolin with a PDE inhibitor. Several PDE inhibitors were used, including both broadly active PDE inhibitors that block PDEs in a non-specific manner as well as specific PDE inhibitors that block only certain PDEs.

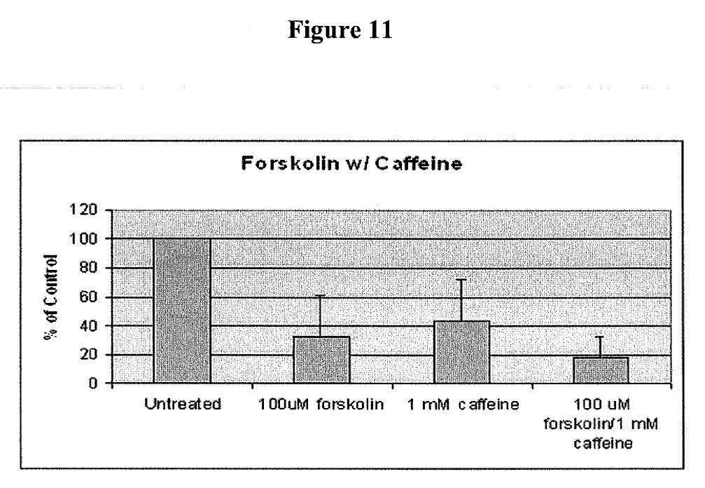

FIG. 11 shows results using caffeine. Caffeine is a bitter white crystalline xanthine alkaloid that acts as a nonspecific PDE inhibitor. It is used as psychoactive stimulant drug and a mild diuretic (speeds up urine production) in humans and other animals.

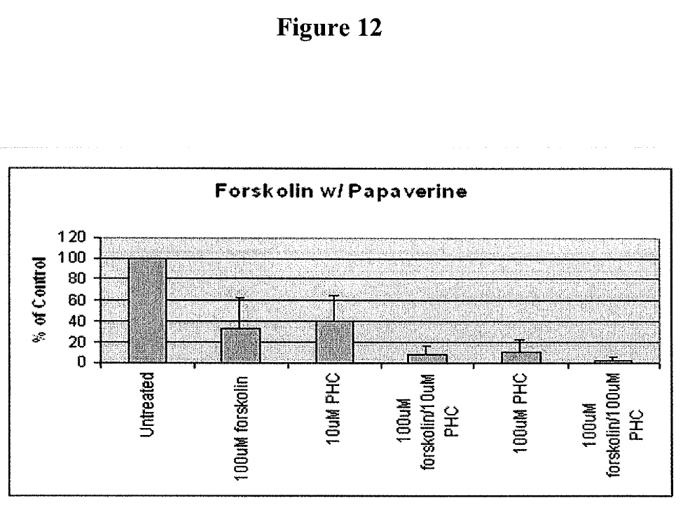

FIG. 12 shows results using papaverine. Papaverine is an opium alkaloid used primarily in the treatment of visceral spasm, vasospasm (especially those involving the heart and the brain), and occasionally in the treatment of erectile dysfunction.

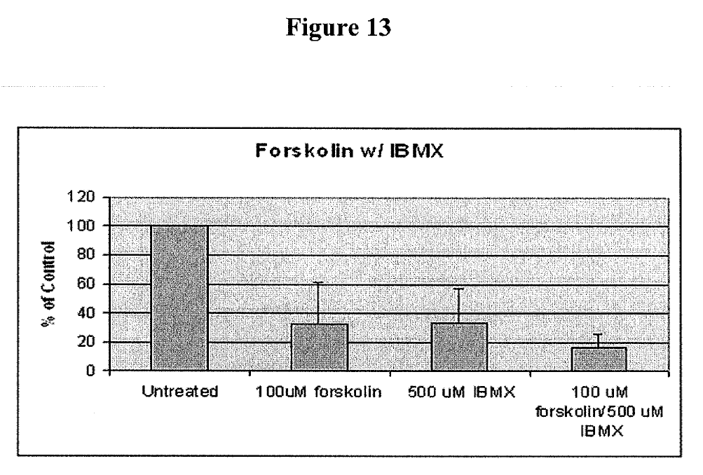

FIG. 13 shows results using isobutylmethylxanthine (IBMX). IBMX is a non-specific inhibitor of phosphodiesterases (IC₅₀=2-50 μM).

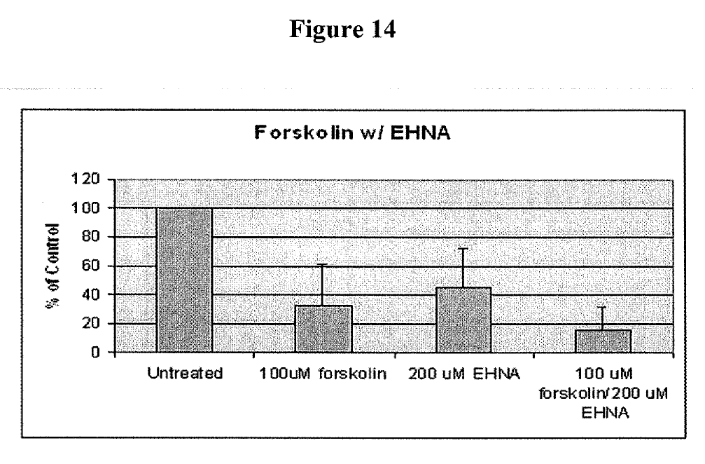

FIG. 14 shows results using erythro-9-(2-hydroxy-3-nonyl)adenine (EHNA) is a PDE2 inhibitor.

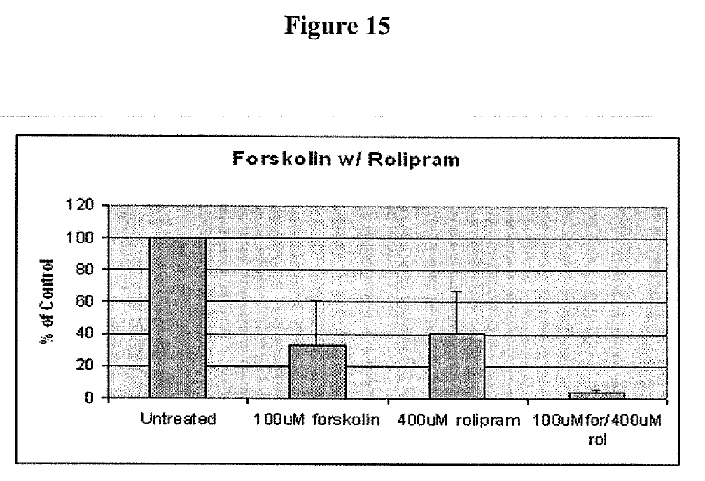

FIG. 15 shows results using rolipram. Rolipram is a PDE4 inhibitor. Like most PDE4 inhibitors, it is an anti-inflammatory drug. Rolipram is being researched as a possible alternative to current antidepressants. Recent studies show that rolipram may have antipsychotic effects. Other beneficial effects of rolipram are improved long term memory, increased wakefulness, and increased neuroprotection.

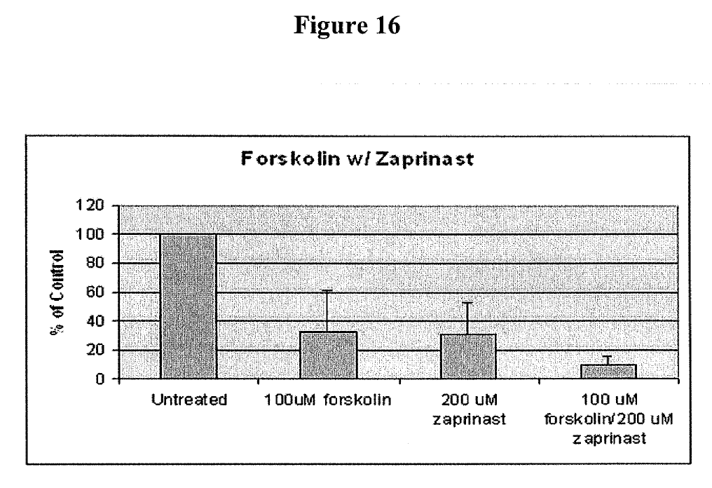

FIG. 16 shows results using zaprinast. Zaprinast a PDE5/6 inhibitor that has been used as treatment for asthma and sexual dysfunction

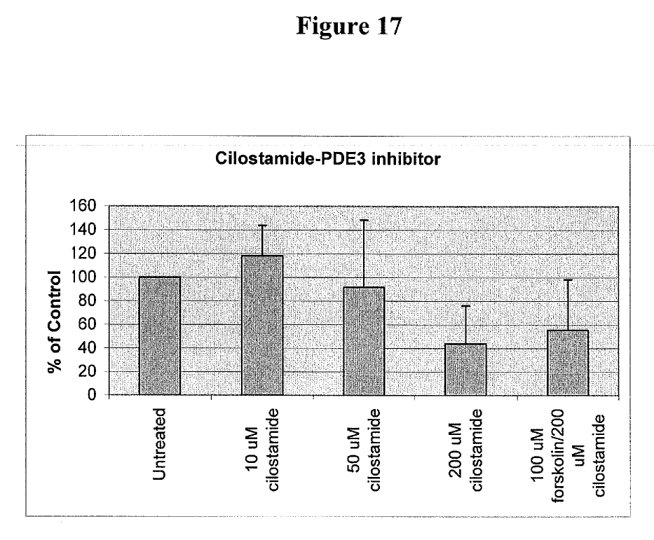

FIG. 17 shows results using cilostamide. Cilostamide (N-Cyclohexyl-N-methyl-4-(1,2-dihydro-2-oxo-6-quinolyloxy)butyramide) is a PDE3 inhibitor.

The data in FIGS. 11 to 17 show that PDE inhibitors, regardless of whether they were nonspecific or specific, were highly effective in reducing bacterial loads in BECs (presented as % of control). In addition, the data show that combination treatments of forskolin with a PDE inhibitor were more effective at limiting bacterial numbers in BECs than single drug treatments.

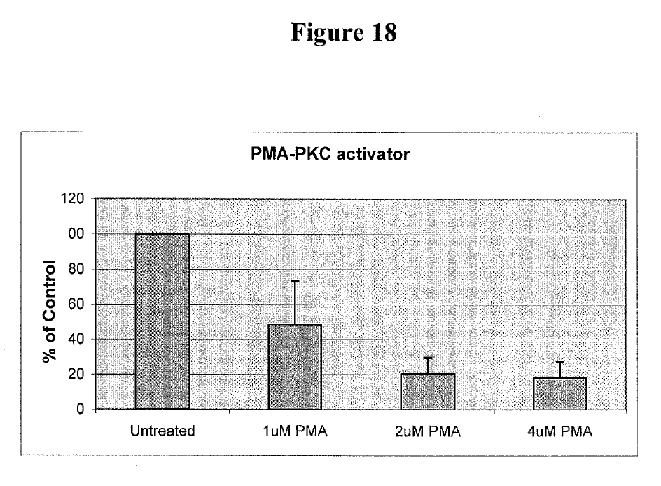

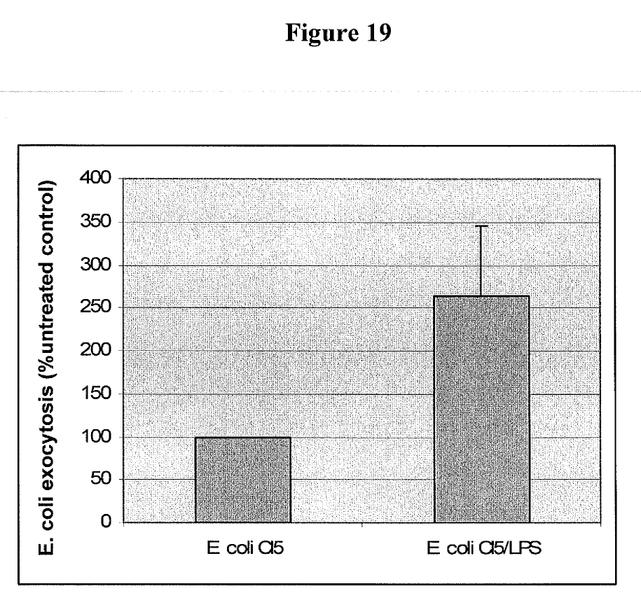

In addition to the above compounds, the effects of Protein Kinase C (PKC) inducers and toll-like receptor 4 ligands were also assessed. Phorbol ester (PMA) is a PKC inducer, and LPS and monophosphoryl lipid A are toll-like receptor 4 ligands. As shown in FIG. 18, PMA significantly reduced intracellular bacterial numbers in BECs. LPS induced greater bacterial exoyctosis from E. coli (CI5) infected BECs than control infected BECs (FIG. 19). Similar results were obtained with monophosphoryl lipid A (data not shown).

Summary

Bacterial load in BECs upon E. coli infection can be reduced by treatment with a wide variety of PDE inhibitors as well as other stimulatory molecules such as PKC activators and toll-like receptor ligands such as lipopolysacchrides and monophosphoryl lipid A. An additive effect was observed when forskolin was combined with PDE inhibitors. A significant advantage to the use of the above mentioned PDE inhibitors is that they have already been approved for use in humans and therefore the fear of toxicity is not an issue.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A method for treating a urinary tract infection comprising administering to a subject in need thereof a therapeutically effective amount of a labdane diterpene or a pharmaceutically acceptable salt thereof.

2. The method of claim 1, wherein said labdane diterpine is a compound of Formula I: 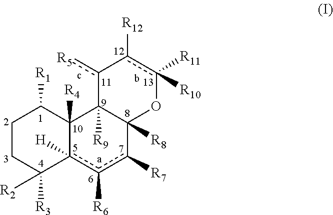
wherein:
a is an optional bond located at the 5, 6 or 6,7 positions, and when present at the 5,6 position, the hydrogen atoms at C₅ and C₆ are absent, and when present at the 6,7 position, the hydrogen atoms at C₆ and C₇ are absent;
b is an optional bond located at the 12,13 position, and when present R₁₁ and R₁₂ are absent;
c is an optional bond between C₁₁ and R₅;
R₁ is selected from the group consisting of H, hydroxyl, —OR₁₃, and —O—C(═O)R₁₄, wherein R₁₃ and R₁₄ are each independently alkyl or substituted alkyl;
R₂, R₃, R₄, R₈, and R₁₀ are each independently selected from the group consisting of alkyl, substituted alkyl, aryl, and substituted aryl;
R₅ is O or S, when c is present, and R₅ is —OR₁₅, when c is absent, wherein R₁₅ is selected from the group consisting of H, alkyl, substituted alkyl, C₁-C₆ carboxylic acyl, and trifluoroacetyl;
R₉ is selected from the group consisting of H, hydroxyl, —OR₁₆, and —O—C(═O)R₁₇, wherein R₁₆ and R₁₇ are each independently alkyl or substituted alkyl; or
R₁ and R₉ together form 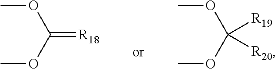
wherein R₁₈ is O or S, and R₁₉ and R₂₀ are each independently selected from the group consisting of H, alkyl, substituted alkyl, alkoxyl, alkenyl, alkynyl, and 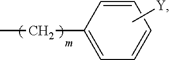
wherein:
m is an integer from 1 to 8; and
Y is selected from the group consisting of H, halogen, alkyl, substituted alkyl, alkoxyl, alkylthio, hydroxyl, —CF₃, —NO₂, —CN, phenyl, benzyl, phenoxy, and NR₂₁R₂₂, wherein R₂₁ and R₂₂ are the same or different and are selected from the group consisting of H, alkyl, and substituted alkyl;
R₁₁ is selected from the group consisting of H, alkyl, substituted alkyl, cycloalkyl, substituted cycloalkyl, —CH₂OH, —C(═O)H, —C(═O)OR₂₃, —CH═CR₂₄R₂₅, and —CCR₂₆,
wherein:
R₂₃, is selected from the group consisting of H, alkyl, and substituted alkyl;
R₂₄ and R₂₅ are each independently selected from the group consisting of H, halogen, —CN, alkyl, substituted alkyl, aryl, substituted aryl, benzyl, substituted benzyl, and —C(═O)(O)ₙR₂₇,
wherein:
n is an integer from 0 to 1;
R₂₇ is selected from the group consisting of H, alkyl, substituted alkyl, aryl, substituted aryl, benzyl, and substituted benzyl;
R₂₆ is H, alkyl, substituted alkyl, alkoxyl, —CHOH—CC—R₂₈, —CH═C═CHR₂₉, —CH═N—OR₃₀, —C(═O)OR₃₁, 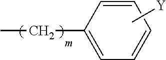
wherein m and Y are as defined above, and 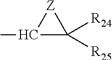
wherein:
m, Y, Z, R₂₄ and R₂₅ are as defined above,
R₂₈, R₂₉, R₃₀, and R₃₁ are each independently selected from the group consisting of H, alkyl, substituted alkyl, aryl, substituted aryl, benzyl, and substituted benzyl,
—CH(ZR₃₂)₂₅
wherein:
Z is as defined above;
R₃₂ is selected from the group consisting of alkyl, substituted alkyl, aryl, substituted aryl, benzyl, substituted benzyl, or the two groups R₃₂ together form —(CH₂)ₙ—, wherein n is an integer from 2 to 3; 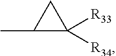
wherein:
R₃₃ and R₃₄ are each independently selected from the group consisting of H, halogen, alkyl, substituted alkyl, aryl, substituted aryl, benzyl, and —C(═O)(O)ₙR₂₇, wherein n and R₂₇ are as defined above; and
—CH═N—NR₃₅R₃₆,
wherein R₃₅ and R₃₆ are each independently selected from the group consisting of H, alkyl, substituted alkyl, aryl, substituted aryl, benzyl, substituted benzyl, —COR₃₇, SO₂R₃₈, and C(═O)OR₃₉, wherein R₃₇, R₃₈, and R₃₉ are selected from the group consisting of alkyl, substituted alkyl, aryl, substituted aryl, benzyl, and substituted benzyl;
R₁₂ is selected from the group consisting of H and halogen;
R₆ and R₇ are the same or different and are selected from the group consisting of H, (═O), —OR₄₀, —O—C(═O)—CR₄₁R₄₂(CH₂)ₚR₄₃, —SO₃OR₄₄ and,
wherein R₄₀ is selected from the group consisting of H, alkyl, substituted alkyl, cycloalkyl, substituted cycloalkyl, cycloheteroalkyl, substituted cycloheteroalkyl, C₁-C₆ carboxylic acyl, 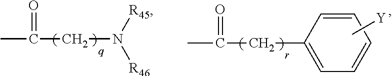
wherein:
p, q, and r are each independently an integer from 0 to 10;
Y is defined as above;
R₄₁ and R₄₂ are each independently selected from the group consisting of H, alkyl, and substituted alkyl;
R₄₃ is selected from the group consisting of H, halogen, alkyl, substituted alkyl, and NR₄₇R₄₈;
R₄₄ is selected from the group consisting of H, alkyl, and substituted alkyl;
R₄₅, R₄₆, R₄₇, and R₄₈ are each independently selected from the group consisting of H, alkyl, substituted alkyl; or
R₄₅ and R₄₆ or R₄₇ and R₄₈ can be combined to form a 3- to 6-membered cycloalkyl or cycloheteroalkyl ring;
R₄₀ is selected from the group consisting of H, alkyl, and substituted alkyl;
or R₆ and R₇ together form a carbonate ester of the following formula: 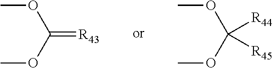
wherein:
R₄₃ is O or S; and
R₄₄ and R₄₅ are each independently selected from the group consisting of H, halogen, alkyl, substituted alkyl, aryl, substituted aryl, benzyl, and —C(═O)(O)₁R₂₇, wherein n and R₂₇ are as defined above;
and pharmaceutically acceptable salts thereof.

3. The method of claim 1, wherein said labdane diterpene is labdane, depicted below.

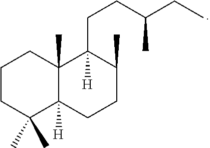

4. The method of claim 1, wherein said labdane diterpene is forskolin, depicted below.

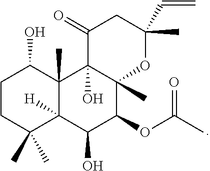

5. The method of claim 1, wherein said labdane diterpene is a forskolin derivative or analog selected from the group consisting of 6-acetyl-7-deacetyl-forskolin, 7-deacetyl-forskolin, 7-deacetyl-6-(N-acetylglycyl)-forskolin, 7-deacetyl-7-O-hemisuccunyl-forskolin, 7-deacetyl-7-(O-N-methylpiperazino)-γ-butryl-dihydrochlonde-forskolin, 7-HPP-forskolin, 6-HPP-forskolin, and colforsin daropate hydrochloride (NKH477).

6. A method for treating a urinary tract infection, the method comprising administering to a subject in need thereof a therapeutically effective amount of an adenylate cyclase activator, a phosphodiesterase (PDE) inhibitor, a Toll-like receptor ligand, a calcium channel activator or calcium activator, a protein kinase A activator, a protein kinase C activator, or adenylate cyclase toxin.

7. The method of claim 6, wherein said adenylate cyclase activator is selected from the group consisting of a labdane diterpene, a G-protein coupled receptor agonist, a G-protein activator, the pyrazole derivative A02011-1, and benzyloxybenzaldehyde and analogs thereof.

8. The method of claim 7, wherein said labdane diterpine is forskolin or a derivative or analog thereof.

9. The method of claim 8, wherein said forskolin derivative or analog is colforsin daropate hydrochloride (NKH477).

10. The method of claim 7, wherein said G-protein coupled receptor agonist is selected from the group consisting of a catecholamine, dopamine, dobutamine, isoproterenol, adenosine, carbacyclin, endothelin, epinephrine, glucagon, octopamine, pituitary adenylate cyclase-activating peptide (PACAP), parathyroid hormone, prostaglandin, and vasopressin.

11. The method of claim 7, wherein said G-protein activator is cholera toxin or a subunit thereof.

12. The method of claim 6, wherein said PDE inhibitor is a cAMP-specific inhibitor.

13. The method of claim 6, wherein said PDE inhibitor is a PDE4 inhibitor.

14. The method of claim 6, wherein said Toll-like receptor ligand is selected from the group consisting of lipopolysaccharide (LPS), 1-palmitoyl-2-linoleoyl-sn-glycero-3-phosphocholine (pLPC), lipoteichoic acid (LTA), and flagellin.

15. The method of claim 6, wherein said calcium channel activator is BAY-K-8644, FPL 64176, or Maitotoxin.

16. The method of claim 6, wherein said calcium activator is a calcium ionophore selected from the group consisting of an ionomycin calcium salt and A23187.

17. The method of claim 6, wherein said calcium activator is the phospholipase C activator 2,4,6-Trimethyl-N-(m-3-trifluoromethylphenyl)benzenesulfonamide.

18. The method of claim 6, wherein said PKA activator is selected from the group consisting of 6-Bnz-cAMP, 8-CPT-2′-O-Me-cAMP, 8-CPT-cAMP, 8-Bromo-cAMP, Dibutyryl-cAMP, Dioctanoyl-cAMP, Sp-8-Br-cAMPS, Sp-cAMPS, cAMP, and a PKA subunit.

19. The method of claim 6, wherein said PKC activator is phorbol myristate acetate (PMA) or a PKC purified enzyme.

20. The method of claim 1, further comprising administering a therapeutically effective amount of an antimicrobial agent to said subject.

21. The method of claim 20, wherein said antimicrobial agent is an antibiotic.

22. The method of claim 21 wherein said antibiotic is selected from the group consisting of a quinolone antibiotic, a cephalosporin antibiotic, a beta-lactam antibiotic, a tetracycline antibiotic, a penicillin antibiotic, a broad-spectrum bactericidal antibiotic, and a bacteriostatic antibiotic.

23. The method of claim 20 wherein said antimicrobial agent is a drug that blocks adherence of bacteria to the bladder wall.

24. The method of claim 23 wherein said drug that blocks adherence of bacteria to the bladder wall is pentosan polysulfate, pentosan polysulfate sodium, D-mannose, 4-methylumbelliferyl alpha-mannoside, or p-nitro-o-chlorophenyl alpha-mannoside.

25. The method of claim 1, further comprising administering a therapeutically effective amount of a cholesterol lowering drug to said subject.

26. The method of claim 25, wherein said cholesterol lowering drug is selected from the group consisting of statins, bile resins, nicotinic acid (niacin), fibric acids (fibrates), and cholesterol absorption inhibitors.

27. A pharmaceutical composition comprising two or more cAMP elevators or agents that mimic cAMP in therapeutically effective amounts for treating a urinary tract infection in a subject in need thereof, and a pharmaceutically acceptable carrier, wherein at least one of said cAMP elevators is a labdane diterpine.

28. The pharmaceutical composition of claim 27, wherein said two or more cAMP elevators or agents that mimic cAMP comprises a first and a second adenylate cyclase activator, wherein said first adenylate cyclase activator is a labdane diterpene and said second adenylate cyclase activator is selected from the group consisting of a G-protein coupled receptor agonist, a G-protein activator, the pyrazole derivative A02011-1 and benzyloxybenzaldehyde and analogs thereof.

29. The pharmaceutical composition of either of claim 27, further comprising an antimicrobial agent in a therapeutically effective amount for treating a urinary tract infection in a subject in need thereof.

30. The pharmaceutical composition of either of claim 27, further comprising a cholesterol lowering drug in a therapeutically effective amount for treating a urinary tract infection in a subject in need thereof.

31. A pharmaceutical composition comprising at least one cAMP elevator or agent that mimics cAMP and at least one additional active compound in therapeutically effective amounts for treating a urinary tract infection in a subject in need thereof, and a pharmaceutically acceptable carrier, wherein said cAMP elevator is a labdane diterpine and said additional active compound is an antimicrobial agent.

32. A pharmaceutical composition comprising at least one cAMP elevator or agent that mimics cAMP and at least one additional active compound in therapeutically effective amounts for treating a urinary tract infection in a subject in need thereof, and a pharmaceutically acceptable carrier, wherein said cAMP elevator is a labdane diterpine and said additional active compound is a cholesterol lowering drug.

33. The pharmaceutical composition of claim 31, wherein said composition comprises a first and a second cAMP elevator, wherein said first cAMP elevator is a labdane diterpine and said second cAMP elevator is a PDE inhibitor.

34. The pharmaceutical composition of claim 33, wherein said PDE inhibitor is a cAMP-specific inhibitor.

35. The pharmaceutical composition of claim 33, wherein said PDE inhibitor is a PDE4 inhibitor.

36. The pharmaceutical composition of either of claim 31, wherein said composition comprises a first and a second cAMP elevator or agent that mimics cAMP, wherein said first cAMP elevator or agent that mimics cAMP is a labdane diterpine and said second cAMP elevator or agent that mimics cAMP is selected from the group consisting of a Toll-like receptor ligand, a calcium channel activator or calcium activator, a protein kinase A activator, a protein kinase C activator, and adenylate cyclase toxin.

37. The pharmaceutical composition of claim 36, wherein said Toll-like receptor ligand is selected from the group consisting of lipopolysaccharide (LPS), 1-palmitoyl-2-linoleoyl-sn-glycero-3-phosphocholine (pLPC), lipoteichoic acid (LTA), and flagellin.

38. The pharmaceutical composition of claim 36, wherein said calcium channel activator is BAY-K-8644, FPL 64176, or Maitotoxin.

39. The pharmaceutical composition of claim 36, wherein said calcium activator is a calcium ionophore selected from the group consisting of an ionomycin calcium salt and A23187.

40. The pharmaceutical composition of claim 36, wherein said calcium activator is the phospholipase C activator 2,4,6-Trimethyl-N-(m-3-trifluoromethylphenyl)benzenesulfonamide.

41. The pharmaceutical composition of claim 36, wherein said PKA activator is selected from the group consisting of 6-Bnz-cAMP, 8-CPT-2′-O-Me-cAMP, 8-CPT-cAMP, 8-Bromo-cAMP, Dibut 1-cAMP, Dioctanoyl-cAMP, Sp-8-Br-cAMPS, Sp-cAMPS, cAMP, and a PKA subunit.

42. The pharmaceutical composition of claim 36, wherein said PKC activator is phorbol myristate acetate (PMA) or a PKC purified enzyme.

43. The pharmaceutical composition of claim 29, wherein said antimicrobial agent is an antibiotic.

44. The pharmaceutical composition of claim 43, wherein said antibiotic is selected from the group consisting of a quinolone antibiotic, a cephalosporin antibiotic, a beta-lactam antibiotic, a tetracycline antibiotic, a penicillin antibiotic, a broad-spectrum bactericidal antibiotic, and a bacteriostatic antibiotic.

45. The pharmaceutical composition of claim 29, wherein said antimicrobial agent is a drug that blocks adherence of bacteria to the bladder wall.

46. The pharmaceutical composition of claim 45, wherein said drug that blocks adherence of bacteria to the bladder wall is pentosan polysulfate, pentosan polysulfate sodium, D-mannose, 4-methylumbelliferyl alpha-mannoside, or p-nitro-o-chlorophenyl alpha-mannoside.

47. The pharmaceutical composition of claim 30, wherein said cholesterol lowering drug is selected from the group consisting of statins, bile resins, nicotinic acid (niacin), fibric acids (fibrates), and cholesterol absorption inhibitors.

48. The pharmaceutical composition of claim 27, wherein said labdane diterpene is selected from the group consisting of forskolin, a forskolin derivative, and a forskolin analog.

49. The pharmaceutical composition of claim 27, wherein said labdane diterpine is a compound of Formula I.

50-52. (canceled)

53. A packaged kit for use in the treatment of a urinary tract infection comprising:
a) a first component comprising a labdane diterpene;
b) a second component comprising an adenylate cyclase activator selected from the group consisting of a G-protein coupled receptor agonist, a G-protein activator, the pyrazole derivative A02011-1, and benzyloxybenzaldehyde and analogs thereof; and
c) instructions for carrying out drug administration of said first and second components in a manner effective to treat said urinary tract infection.

54. A packaged kit for use in the treatment of a urinary tract infection comprising:
a) a first component comprising a labdane diterpene;
b) a second component comprising a phosophodiesterase (PDE) inhibitor; and
c) instructions for carrying out drug administration of said first and second components in a manner effective to treat said urinary tract infection.

55-56. (canceled)

57. A packaged kit for use in the treatment of a urinary tract infection comprising:
a) a first component comprising a labdane diterpene;
b) a second component comprising a cAMP elevator or agent that mimics cAMP selected from the group consisting of a Toll-like receptor ligand, a calcium channel activator or calcium activator, a protein kinase A activator, a protein kinase C activator, and adenylate cyclase toxin; and
c) instructions for carrying out drug administration of said first and second components in a manner effective to treat said urinary tract infection.

58-63. (canceled)

64. The packaged kit of claim 53, further comprising a third component comprising an antimicrobial agent, wherein said instructions further comprise instructions for carrying out drug administration of said first, second, and third components in a manner effective to treat said urinary tract infection.

65. A packaged kit for use in the treatment of a urinary tract infection comprising:
a) a first component comprising a labdane diterpene;
b) a second component comprising an antimicrobial agent; and
c) instructions for carrying out drug administration of said first and second components in a manner effective to treat said urinary tract infection.

66-69. (canceled)

70. The packaged kit of claim 53, further comprising a third component comprising a cholesterol lowering drug, wherein said instructions further comprise instructions for carrying out drug administration of said first, second, and third components in a manner effective to treat said urinary tract infection.

71. A packaged kit for use in the treatment of a urinary tract infection comprising:
a) a first component comprising a labdane diterpene;
b) a second component comprising a cholesterol lowering drug; and
c) instructions for carrying out drug administration of said first and second components in a manner effective to treat said urinary tract infection.

72-76. (canceled)

77. The packaged kit of claim 53, wherein said components are contained in the same pharmaceutical formulation.

78. The packaged kit of claim 53, wherein said components are contained in separate pharmaceutical formulations.

79. The packaged kit of claim 53, wherein said instructions include directions for carrying out drug administration of said components sequentially or concurrently.
